(19) United States
(12) Patent Application Publication
(10) Pub. No.: US 2010/0125081 A1
(43) Pub. Date: May 20, 2010

(54) NEW COMPOUNDS 574

(75) Inventors: Jorg HOLENZ, Sodertalje (SE); Sofia KARLSTROM, Sodertalje (SE); Jacob KIHLSTROM, Sodertalje (SE); Karin KOLMODIN, Sodertalje (SE); Johan LINDSTRÖM, Sodertalje (SE); Laszlo RAKOS, Sodertalje (SE); Didier ROTTICCI, Sodertalje (SE); Peter SODERMAN, Sodertalje (SE); Marie SUNDSTROM, Sodertalje (SE); Britt-Marie SWAHN, Sodertalje (SE); Stefan VON BERG, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/618,088

(22) Filed: Nov. 13, 2009

Related U.S. Application Data
(60) Provisional application No. 61/114,608, filed on Nov. 14, 2008.

(51) Int. Cl. C07D 471/04; A61K 31/437; A61K 31/506; A61K 31/497; C12N 9/99; A61P 25/28; A61P 25/00

(52) U.S. Cl. 514/255.05; 546/113; 514/300; 514/256; 544/333; 544/405; 435/184

(57) ABSTRACT

The present invention relates to novel compounds of formula (I) and their pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

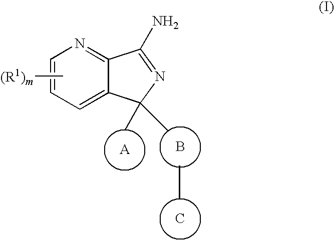

### TECHNICAL FIELD OF THE INVENTION

The present invention relates to novel compounds and their pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class 1 transmembrane protein called APP, or amyloid precursor protein. Cleavage of APP by BACE generates the extracellular soluble APPβ fragment and the membrane bound CTFβ (C99) fragment that is subsequently is cleaved by γ-secretase to generate Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products-amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much higher risk of developing AD, and also of developing the disease at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to develop Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of Aβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors, see e.g WO2001/00665, WO2005/058311, WO2006/138265, WO2009005471, WO2009005470, WO2007149033 and WO2009022961.

### OUTLINE OF THE INVENTION

The present invention relates to a compound according to formula (I):

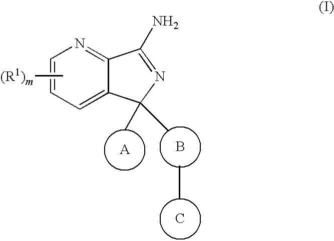

wherein
R¹ is selected from halogen, cyano, NO₂, SO₂R², C₁₋₆alkyl, C₂₋₆alkenyl, C₂₋₆alkynyl, NR³R⁴, OR², C(O)R², C(O)NR³R⁴ and COOR², wherein said C₁₋₆alkyl, C₂₋₆alkenyl or C₂₋₆alkynyl is optionally substituted with one or more R⁷;
R² is C₁₋₆alkyl, C₂₋₆alkenyl or C₂₋₆alkynyl, wherein said C₁₋₆alkyl, C₂₋₆alkenyl or C₂₋₆alkynyl is optionally substituted with one or more R⁷;
R³ and R⁴ are independently selected from hydrogen, C₁₋₆alkyl, C₂₋₆alkenyl, C₂₋₆alkynyl, aryl, heteroaryl, heterocyclyl and carbocyclyl, wherein said C₁₋₆alkyl, C₂₋₆alkenyl, C₂₋₆alkynyl, aryl, heteroaryl, heterocyclyl or carbocyclyl is optionally substituted with one or more R⁷;
or R³ and R⁴ together with the atom they are attached to form a 4 to 7 membered ring;
A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
C is selected from hydrogen, halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein said aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl or C₂₋₆alkenylC₃₋₆cycloalkyl is optionally substituted with one to three R⁷;
R⁵ is selected from halo, cyano, C₁₋₆alkyl, C₁₋₆haloalkyl, C₃₋₆cycloalkyl, OC₁₋₆alkyl, OC₂₋₆alkenyl and OC₁₋₆alkylaryl, wherein said C₁₋₆alkyl, C₁₋₆haloalkyl, C₃₋₆cycloalkyl, OC₁₋₆alkyl, OC₂₋₆alkenyl or OC₁₋₆alkylaryl is optionally substituted with one to three R⁷;
R⁶ is halogen, hydroxy, or cyano;
R⁷ is selected from halogen, cyano, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, C(O)OC₁₋₃alkyl and NR⁸R⁹, wherein said C₁₋₆alkyl, SO₂C₁₋₃alkyl, OC₁₋₃alkyl, OC₁₋₃haloalkyl, C₁₋₃alkylOH, C₁₋₃alkylNR⁸R⁹ or C(O)OC₁₋₃alkyl is optionally substituted with one or more R¹⁰;
R⁸ and R⁹ are independently selected from hydrogen, C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl and carbocyclyl, wherein said C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl or carbocyclyl is optionally substituted with one or more R¹⁰;
or R⁸ and R⁹ together with the atom they are attached to form a 4 to 6 membered ring;
R¹¹ is selected from halo, C₁₋₃alkyl, OC₁₋₃alkyl and OC₁₋₃haloalkyl;
R¹¹ and R¹² are independently selected from hydrogen, C₁₋₃alkyl and C₁₋₃haloalkyl;
m is 0, 1 or 2;
as a free base or a pharmaceutically acceptable salt thereof.

Another aspect of the present invention relates to a compound of formula (I), wherein

R¹ is selected from halogen, cyano, NO₂, SO₂R², C₁₋₆alkyl, C₂₋₆alkenyl, C₂₋₆alkynyl, NR³R⁴, OR², C(O)R², C(O)NR³R⁴ and COOR², wherein said C₁₋₆alkyl, C₂₋₆alkenyl or C₂₋₆alkynyl is optionally substituted with one or more R⁷;
R² is C₁₋₆alkyl, C₂₋₆alkenyl or C₂₋₆alkynyl, wherein said C₁₋₆alkyl, C₂₋₆alkenyl or C₂₋₆alkynyl is optionally substituted with one or more R⁷;
R³ and R⁴ are independently selected from hydrogen, C₁₋₆alkyl, C₂₋₆alkenyl, C₂₋₆alkynyl, aryl, heteroaryl, heterocyclyl and carbocyclyl, wherein said C₁₋₆alkyl, C₂₋₆alkenyl, C₂₋₆alkynyl, aryl, heteroaryl, heterocyclyl or carbocyclyl is optionally substituted with one or more R⁷;
or R³ and R⁴ together with the atom they are attached to form a 4 to 7 membered ring;
A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
C is selected from halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein said aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl or C₂₋₆alkenylC₃₋₆cycloalkyl is optionally substituted with one to three R⁷;

R⁵ is selected from halo, cyano, C₁₋₆alkyl, C₁₋₆haloalkyl, C₃₋₆cycloalkyl, OC₁₋₆alkyl, OC₂₋₆alkenyl and OC₁₋₆alkylaryl, wherein said C₁₋₆alkyl, C₁₋₆haloalkyl, C₃₋₆cycloalkyl, OC₁₋₆alkyl, OC₂₋₆alkenyl or OC₁₋₆alkylaryl is optionally substituted with one to three R⁷;

R⁶ is halogen, hydroxy or cyano;
R⁷ is selected from halogen, cyano, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, C(O)OC₁₋₃alkyl and NR⁸R⁹, wherein said C₁₋₆alkyl, SO₂C₁₋₃alkyl, OC₁₋₃alkyl, OC₁₋₃haloalkyl, C₁₋₃alkylOH, C₁₋₃alkylNR⁸R⁹ or C(O)OC₁₋₃alkyl is optionally substituted with one or more R¹⁰;
R⁸ and R⁹ are independently selected from hydrogen, C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl and carbocyclyl, wherein said C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl or carbocyclyl is optionally substituted with one or more R¹⁰;
or R⁸ and R⁹ together with the atom they are attached to form a 4 to 6 membered ring;
R¹⁰ is selected from halo, C₁₋₃alkyl, OC₁₋₃alkyl and OC₁₋₃haloalkyl;
R¹¹ and R¹² are independently selected from hydrogen, C₁₋₃allyl and C₁₋₃haloalkyl;
m is 0, 1 or 2;
as a free base or a pharmaceutically acceptable salt thereof.

One embodiment of the present invention, relates to a compound of formula (I), wherein

R¹ is selected from halogen, cyano, NO₂, SO₂R², C₁₋₆alkyl, NR³R⁴, OR², C(O)R², C(O)NR³R⁴ and COOR², wherein said C₁₋₆alkyl is optionally substituted with one or more R⁷;
R² is C₁₋₆alkyl, C₂₋₆alkenyl or C₂₋₆alkynyl, wherein said C₁₋₆alkyl, C₂₋₆alkenyl or C₂₋₆alkynyl is optionally substituted with one or more R⁷;
R³ and R⁴ are independently selected from hydrogen, C₁₋₆alkyl, aryl, heteroaryl, heterocyclyl and carbocyclyl, wherein said C₁₋₆alkyl, aryl, heteroaryl, heterocyclyl or carbocyclyl is optionally substituted with one or more R⁷;
or R³ and R⁴ together with the atom they are attached to form a 4 to 7 membered ring;
A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
C is selected from halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein said aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl or C₂₋₆alkenylC₃₋₆cycloalkyl is optionally substituted with one to three R⁷;
R⁵ is selected from halo, cyano, C₁₋₆alkyl, C₁₋₆haloalkyl, C₃₋₆cycloalkyl, OC₁₋₆alkyl, OC₂₋₆alkenyl and OC₁₋₆alkylaryl, wherein said C₁₋₆alkyl, C₁₋₆haloalkyl, C₃₋₆cycloalkyl, OC₁₋₆alkyl, OC₂₋₆alkenyl or OC₁₋₆alkylaryl is optionally substituted with one to three R⁷;
R⁶ is halogen, hydroxy or cyano;
R⁷ is selected from halogen, cyano, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, cyano and C(O)OC₁₋₃alkyl, wherein said C₁₋₆alkyl, SO₂C₁₋₃alkyl, OC₁₋₃alkyl, OC₁₋₃haloalkyl, C₁₋₃alkylOH, C₁₋₃alkylNR⁸R⁹ or C(O)OC₁₋₃alkyl is optionally substituted with one or more R¹⁰;
R⁸ and R⁹ are independently selected from hydrogen, C₁₋₆alkyl, C₁₋₆haloalkyl, C₁₋₃alkylNR¹¹R¹², C₁₋₃alkylOaryl, heteroaryl, heterocyclyl and carbocyclyl, wherein said C₁₋₆alkyl, C₁₋₆haloalkyl, C₁₋₃alkylNR¹¹R¹², C₁₋₃alkylOaryl, heteroaryl, heterocyclyl or carbocyclyl is optionally substituted with one or more R¹⁰;
or R⁸ and R⁹ together with the atom they are attached to form a 4 to 6 membered ring;
R¹⁰ is selected from halo, C₁₋₃alkyl, OC₁₋₃alkyl and OC₁₋₃haloalkyl;
R¹¹ and R¹² are independently selected from hydrogen, C₁₋₃alkyl and C₁₋₃haloalkyl;
m is 0, 1 or 2.

One embodiment of the present invention, relates to a compound of formula (I), wherein

R¹ is selected from halogen, cyano, NO₂, SO₂R², C₁₋₆alkyl, NR³R⁴, OR² and C(O)R², wherein said C₁₋₆alkyl is optionally substituted with one or more R⁷;
R² is C₁₋₆alkyl, optionally substituted with one or more R⁷;
R³ and R⁴ are independently selected from hydrogen, C₁₋₆alkyl, aryl, heteroaryl, heterocyclyl and carbocyclyl, wherein said C₁₋₆alkyl, aryl, heteroaryl, heterocyclyl or carbocyclyl is optionally substituted with one or more R⁷;
A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
C is selected from halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein said aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl or C₂₋₆alkenylC₃₋₆cycloalkyl is optionally substituted with one to three R⁷;
R⁵ is selected from halo, cyano, C₁₋₆alkyl, C₁₋₆haloalkyl, C₃₋₆cycloalkyl, OC₁₋₆alkyl, OC₂₋₆alkenyl and OC₁₋₆alkylaryl, wherein said C₁₋₆alkyl, C₁₋₆haloalkyl, C₃₋₆cycloalkyl, OC₁₋₆alkyl, OC₂₋₆alkenyl or OC₁₋₆alkylaryl is optionally substituted with one to three R⁷;
R⁶ is halogen or hydroxy;
R⁷ is selected from halogen, cyano, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, cyano and C(O)OC₁₋₃alkyl, wherein said C₁₋₆alkyl, SO₂C₁₋₃alkyl, OC₁₋₃alkyl, OC₁₋₃haloalkyl, C₁₋₃alkylOH, C₁₋₃alkylNR⁸R⁹ or C(O)OC₁₋₃alkyl is optionally substituted with one or more R¹⁰;
R⁸ and R⁹ are independently selected from hydrogen, C₁₋₆alkyl, C₁₋₆haloalkyl, C₁₋₃alkylNR¹¹R¹², C₁₋₃alkylOaryl, heteroaryl, heterocyclyl and carbocyclyl, wherein said C₁₋₆alkyl, C₁₋₆haloalkyl, C₁₋₃alkylNR¹¹R¹², C₁₋₃alkylOaryl, heteroaryl, heterocyclyl or carbocyclyl is optionally substituted with one or more R¹⁰;
R¹⁰ is selected from halo, C₁₋₃alkyl, OC₁₋₃alkyl and OC₁₋₃haloalkyl;
R¹¹ and R¹² are independently selected from hydrogen, C₁₋₃alkyl and C₁₋₃haloalkyl; m is 0 or 1.

One embodiment of the present invention, relates to a compound of formula (I), wherein A is heteroaryl. According to another embodiment of the present invention, said heteroaryl is pyridinyl or pyrimidine.

One embodiment of the present invention, relates to a compound of formula (I), wherein A is aryl. According to another embodiment of the present invention, said aryl is phenyl.

One embodiment of the present invention, relates to a compound of formula (I), wherein A is not substituted.

One embodiment of the present invention, relates to a compound of formula (I), wherein A is substituted with one or more R⁵.

One embodiment of the present invention, relates to a compound of formula (I), wherein C is selected from halogen, cyano, aryl, heteroaryl, heterocyclyl, C₃₋₆cycloalkyl, C₁₋₆alkyl, C₁₋₆alkylC₃₋₆heterocyclyl, C₁₋₆alkylaryl and C₁₋₆alkylheteroaryl.

One embodiment of the present invention, relates to a compound of formula (I), wherein C is selected from halogen, cyano, aryl, heteroaryl and C₁₋₆alkyl.

One embodiment of the present invention, relates to a compound of formula (I), wherein C is not substituted.

One embodiment of the present invention, relates to a compound of formula (I), wherein C is substituted with one to three R⁷. According to another embodiment of the present invention, R⁷ is selected from halogen, cyano, C₁₋₆alkyl, SO₂C₁₋₃alkyl, OC₁₋₃alkyl and OC₁₋₃haloalkyl.

One embodiment of the present invention, relates to a compound of formula (I), R⁶ is fluoro, chloro or hydroxy. According to another embodiment of the present invention, R⁶ is fluoro.

One embodiment of the present invention, relates to a compound of formula (I), wherein m is 0.

One embodiment of the present invention, relates to a compound of formula (I), wherein

A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
C is selected from halogen, cyano, aryl, heteroaryl and C₁₋₆alkyl, wherein said aryl, heteroaryl or C₁₋₆alkyl is optionally substituted with one to three R⁷;
R⁵ is selected from halo, cyano, C₁₋₆alkyl, C₁₋₆haloalkyl, C₃₋₆cycloalkyl, OC₂₋₆alkenyl and
OC₁₋₆alkylaryl, wherein said C₁₋₆alkyl, C₁₋₆haloalkyl, C₃₋₆cycloalkyl, OC₂₋₆alkenyl or OC₁₋₆alkylaryl is optionally substituted with one to three R⁷;
R⁶ is halogen or hydroxy;
R⁷ is selected from halogen, cyano, C₁₋₆alkyl, SO₂C₁₋₃alkyl, OC₁₋₃alkyl, OC₁₋₃haloalkyl, wherein said C₁₋₆alkyl, SO₂C₁₋₃alkyl, OC₁₋₃alkyl or OC₁₋₃haloalkyl is optionally substituted with one or more R¹⁰;
R¹⁰ is halo.
m is 0 or 1.

One embodiment of the present invention, relates to a compound of formula (I), wherein

A is heteroaryl, wherein said heteroaryl is optionally substituted with one or more R⁵;
B is aryl;
C is selected from aryl and heteroaryl, wherein said aryl or heteroaryl is optionally substituted with one to three R⁷;
R⁵ is selected from C₁₋₆alkyl, OC₂₋₆alkenyl and C₁₋₆haloalkyl, wherein said C₁₋₆alkyl or OC₂₋₆alkenyl is optionally substituted with one to three R⁷;
R⁷ is selected from halogen, cyano;
m is 1.

According to one embodiment of the present invention, B is phenyl.

In one embodiment of the present invention, R⁵ is selected from halo, cyano, C₁₋₆alkyl, C₁₋₆haloalkyl, C₃₋₆cycloalkyl, OC₁₋₆alkyl and OC₁₋₆alkylaryl, wherein said C₁₋₆alkyl, C₁₋₆haloalkyl, C₃₋₆cycloalkyl, OC₁₋₆alkyl or OC₁₋₆alkylaryl is optionally substituted with one to three R⁷.

In one embodiment of the present invention, R⁶ is halogen or cyano.

The present invention also relates to a compound selected from:
5-(3′-chlorobiphenyl-3-yl)-5-(2-(trifluoromethyl)pyridin-4-yl)-5H-pyrrolo[3,4-b]pyridin-7-amine;
5-(3-(pyridin-3-yl)phenyl)-5-(2-(trifluoromethyl)pyridin-4-yl)-5H-pyrrolo[3,4-b]pyridin-7-amine;
5-(3-(pyridin-3-yl)phenyl)-5-(2-(trifluoromethyl)pyridin-4-yl)-5H-pyrrolo[3,4-b]pyridin-7-amine;
5-(2,6-dimethylpyridin-4-yl)-5-(3-(pyrimidin-5-yl)phenyl)-5H-pyrrolo[3,4-b]pyridin-7-amine;
5-(3-(7-amino-5-(2,6-dimethylpyridin-4-yl)-5H-pyrrolo[3,4-b]pyridin-5-yl)phenyl)nicotinonitrile;
5-(3,5-difluoro-4-methoxyphenyl)-5-(4-fluoro-3-(pyrimidin-5-yl)phenyl)-5H-pyrrolo[3,4-b]pyridin-7-amine;
5-(3-chloro-4-methoxyphenyl)-5-(3-(pyrimidin-5-yl)phenyl)-5H-pyrrolo[3,4-b]pyridin-7-amine;
5-(3-chloro-4-methoxyphenyl)-5-(3-(pyrazin-2-yl)phenyl)-5H-pyrrolo[3,4-b]pyridin-7-amine;
5-(3-cyclopropyl-4-(difluoromethoxy)phenyl)-5-(3-(pyrimidin-5-yl)phenyl)-5H-pyrrolo[3,4-b]pyridin-7-amine;
3-chloro-5-(2-methylpyridin-4-yl)-5-(3-(pyrimidin-5-yl)phenyl)-5H-pyrrolo[3,4-b]pyridin-7-amine;
5-(4-methoxyphenyl)-3-methyl-5-(3-(pyrimidin-5-yl)phenyl)-5H-pyrrolo[3,4-b]pyridin-7-amine;
5-(4-(difluoromethoxy)-3,5-dimethylphenyl)-5-(3-(pyrimidin-5-yl)phenyl)-5H-pyrrolo[3,4-b]pyridin-7-amine;
5-(4-(difluoromethoxy)-3,5-dimethylphenyl)-5-(3-(pyrazin-2-yl)phenyl)-5H-pyrrolo[3,4-b]pyridin-7-amine;
5-(4-fluoro-3,5-dimethylphenyl)-5-(3-(pyrimidin-5-yl)phenyl)-5H-pyrrolo[3,4-b]pyridin-7-amine;
5-(3-fluoro-4-methoxy-5-methylphenyl)-5-(3-(pyrazin-2-yl)phenyl)-5H-pyrrolo[3,4-b]pyridin-7-amine;
5-(3-(pyrimidin-5-yl)phenyl)-5-(2-(2,2,2-trifluoroethoxy)pyridin-4-yl)-5H-pyrrolo[3,4-b]pyridin-7-amine;
5-(2-(2,2-difluorovinyloxy)pyridin-4-yl)-5-(3-(pyrimidin-5-yl)phenyl)-5H-pyrrolo[3,4-b]pyridin-7-amine;
5-(4-(difluoromethoxy)-3-fluorophenyl)-5-(3-(pyrimidin-5-yl)phenyl)-5H-pyrrolo[3,4-b]pyridin-7-amine;
5-(3-(4-methoxypyridin-2-yl)phenyl)-5-(2-(trifluoromethyl)pyridin-4-yl)-5H-pyrrolo[3,4-b]pyridin-7-amine;
5-(2-(difluoromethyl)-6-methylpyridin-4-yl)-5-(3-(pyrimidin-5-yl)phenyl)-5H-pyrrolo[3,4-b]pyridin-7-amine;
5-(3-(5-chloropyridin-3-yl)phenyl)-5-(2-(trifluoromethyl)pyridin-4-yl)-5H-pyrrolo[3,4-b]pyridin-7-amine;
2-(3-(7-dmino-5-(2-(trifluoromethyl)pyridin-4-yl)-5H-pyrrolo[3,4-b]pyridin-5-yl)phenyl)isonicotinonitrile;
5-(3-(difluoromethyl)-4-methoxyphenyl)-5-(3-(pyrazin-2-yl)phenyl)-5H-pyrrolo[3,4-b]pyridin-7-amine;
5-(3-(difluoromethyl)-4-methoxyphenyl)-5-(3-(pyrimidin-5-yl)phenyl)-5H-pyrrolo[3,4-b]pyridin-7-amine;
5-(4-(fluoromethoxy)-3,5-dimethylphenyl)-5-(3-(pyrimidin-5-yl)phenyl)-5H-pyrrolo[3,4-b]pyridin-7-amine;
5-(4-(fluoromethoxy)-3,5-dimethylphenyl)-5-(3-(pyrazin-2-yl)phenyl)-5H-pyrrolo[3,4-b]pyridin-7-amine;
5-(2-(3-fluoropropoxy)pyridin-4-yl)-5-(3-(pyrimidin-5-yl)phenyl)-5H-pyrrolo[3,4-b]pyridin-7-amine;
5-(4-difluoromethoxy-3,5-dimethyl-phenyl)-5-(2-pyrimidin-5-yl-pyridin-4-yl)-5H-pyrrolo[3,4-b]pyridin-7-ylamine;
5-(3-cyclopropyl-4-difluoromethoxy-5-methyl-phenyl)-5-(2-pyrimidin-5-yl-pyridin-4-yl)-5H-pyrrolo[3,4-b]pyridin-7-ylamine;
5-[3-cyclopropyl-4-(difluoromethoxy)-5-methyl-phenyl]-5-phenyl-pyrrolo[3,4-b]pyridin-7-amine;
3-[7-amino-5-(3-cyclopropyl-4-difluoromethoxy-5-methyl-phenyl)-5H-pyrrolo[3,4-b]pyridin-5-yl]-benzonitrile;
5-(3-cyclopropyl-4-methoxy-phenyl)-5-(3-pyrimidin-5-yl-phenyl)-5H-pyrrolo[3,4-b]pyridin-7-ylamine;
5-[4-difluoromethoxy-3-(2-fluoro-ethyl)-phenyl]-5-(3-pyrimidin-5-yl-phenyl)-5H-pyrrolo[3,4-b]pyridin-7-ylamine;
5-(5-methoxy-4,6-dimethyl-pyridin-2-yl)-5-(3-pyrimidin-5-yl-phenyl)-5H-pyrrolo[3,4-b]pyridin-7-ylamine;
5-(3-fluoro-4-methoxy-5-methylphenyl)-5-(3-(pyrimidin-5-yl)phenyl)-5H-pyrrolo[3,4-b]pyridin-7-amine;
5-(7-amino-5-(2-(trifluoromethyl)pyridin-4-yl)-5H-pyrrolo[3,4-b]pyridin-5-yl)-2′-fluoro-5′-methoxybiphenyl-2-ol; and
5-(7-amino-5-(2-(trifluoromethyl)pyridin-4-yl)-5H-pyrrolo[3,4-b]pyridin-5-yl)-2′-fluorobiphenyl-2-ol
   as a free base or a pharmaceutically acceptable salt thereof.

In another aspect of the invention, there is provided a pharmaceutical composition comprising as active ingredient a therapeutically effective amount of a compound according formula (I) in association with pharmaceutically acceptable excipients, carriers or diluents.

In another aspect of the invention, there is provided a compound according to formula (I), or a pharmaceutically acceptable salt thereof, for use as a medicament.

In another aspect of the invention, there is provided use of a compound according to formula (I), as a medicament for treating or preventing an Aβ-related pathology.

In another aspect of the invention, there is provided use of a compound according to formula (I), as a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer Disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing an Aβ-related pathology in a mammal, such as a human, comprising administering to said patient a therapeutically effective amount of a compound according to formula (I), and at least one cognitive enhancing agent, memory enhancing agent, or choline esterase inhibitor, wherein said Aβ-related pathology is Alzheimer Disease.

The present invention relates to the use of compounds of formula (I) as hereinbefore defined as well as to the salts thereof. Salts for use in pharmaceutical compositions will be pharmaceutically acceptable salts, but other salts may be useful in the production of the compounds of formula (I)

It is to be understood that the present invention relates to any and all tautomeric forms of the compounds of formula (I).

Compounds of the invention can be used as medicaments. In some embodiments, the present invention provides compounds of formula (I), or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy, traumatic brain injury or cortical basal degeneration.

In some embodiments, the present invention provides use of compounds of formula (I) or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula (I), or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula (I) or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula (I) or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula (I) or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the invention has been diagnosed with a particular disease or disorder, such as those described herein. In these cases, the mammal or human being treated is in need of such treatment. Diagnosis, however, need not be previously performed.

The present invention also includes pharmaceutical compositions, which contain, as the active ingredient, one or more of the compounds of the invention herein together with at least one pharmaceutically acceptable carrier, diluent or excipient.

The definitions set forth in this application are intended to clarify terms used throughout this application. The term “herein” means the entire application.

All compounds in the present invention may exist in particular geometric or stereo isomeric forms. The present invention takes into account all such compounds, including cis- and trans isomers, R- and S-enantiomers, diastereomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used in this application, the term “optionally substituted,” means that substitution is optional and therefore it is possible for the designated atom or moiety to be unsubstituted.

In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is is methyl (i.e., CH₃), then 3 hydrogens on the carbon atom can be replaced. Examples of such substituents include, but are not limited to: halo, CN, NH₂, OH, COOH, OC₁₋₆alkyl, C₁₋₆alkylOH, SO₂H, C₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, N(C₁₋₆alkyl)C(O)C₁₋₆alkyl, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)_{z}, NHC(O)aryl, NarylC(O)aryl, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)_{z}, NHC(O)heteroaryl, NheteroarylC(O)heteroaryl, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(O)C₅₋₆heterocyclyl, NC₅₋₆heterocyclyl C(O)C₅₋₆heterocyclyl.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used also or as a suffix or prefix is intended to include both branched and straight-chain alkynyl or olefin containing aliphatic hydrocarbon groups having from 2 to 12 carbon atoms or if a specified number of carbon atoms is provided then that specific number would be intended. Examples include, but are not limited to, ethynyl, 1-propynyl, 2-propynyl, 3-butynyl, pentynyl, hexynyl and 1-methylpent-2-ynyl.

As used herein, “aromatic” refers to hydrocarbonyl groups having one or more unsaturated carbon ring(s) having aromatic characters, (e.g. 4 n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4 n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. Examples of polycyclic rings include, but are not limited to, 2,3-dihydro-1,4-benzodioxine and 2,3-dihydro-1-benzofuran.

As used herein, the term “cycloalkyl” or “carbocyclyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, the term “cycloalkenyl” is intended to include unsaturated ring groups, is having the specified number of carbon atoms. These may include fused or bridged polycyclic systems. Preferred cycloalkenyls have from 3 to 10 carbon atoms in their ring structure, and more preferably have 3, 4, 5, and 6 carbons in the ring structure. For example, “C₃₋₆ cycloalkenyl” denotes such groups as cyclopropenyl, cyclobutenyl, cyclopentenyl, or cyclohexenyl.

As used herein, “halo” or “halogen” refers to fluoro, chloro, bromo, and iodo.

As used herein, “haloalkyl”, used alone or as a suffix or prefix, is intended to include both branched and straight chain saturated aliphatic hydrocarbon groups, having at least one halogen substituent and having from 1 to 12 carbon atoms or if a specified number of carbon atoms is provided then that specific number would be intended. For example “C₀₋₆haloalkyl” denotes alkyl having 0, 1, 2, 3, 4, 5 or 6 carbon atoms. Examples of haloalkyl include, but are not limited to, fluoromethyl, difluoromethyl, trifluoromethyl, chlorofluoromethyl, 1-fluoroethyl, 3-fluoropropyl, 2-chloropropyl, 3,4-difluorobutyl.

“Counterion” is used to represent a small, negatively or positively charged species such as chloride, bromide, hydroxide, acetate, sulfate, tosylate, benezensulfonate, ammonium, lithium ion and sodium ion and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring NH is optionally substituted with acetyl, formyl, methyl or mesyl; and a ring is optionally substituted with one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is a non-aromatic heterocycle. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, benzoxazolyl, aza-benzoxazolyl, indolinyl, imidazothiazolyl and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the non-toxic salts or the quaternary ammonium salts of the parent compound formed, for example, from non-toxic inorganic or organic acids. For example, such non-toxic salts include those derived from inorganic acids such as hydrochloric acid.

The pharmaceutically acceptable salts of the present invention can be synthesized from the parent compound that contains a basic or acidic moiety by conventional chemical methods.

Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labeled compounds of the invention. An “isotopically” or “radio-labeled” compound is a compound of the invention where one or more atoms are replaced or substituted with an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium) ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

For the avoidance of doubt the present invention relates to any one of compounds falling within the scope of formula (I) as defined above.

It will be appreciated that throughout the specification, the number and nature of substituents on rings in the compounds of the invention will be selected so as to avoid sterically undesirable combinations.

The anti-dementia treatment defined herein may be applied as a sole therapy or may involve, in addition to the compound of the invention, conventional therapy. Such therapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Additional conventional therapy may include one or more of the following categories of agents:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof.

(ii) atypical antipsychotics including for example quetiapine and pharmaceutically active isomer(s) and metabolite(s) thereof.

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof.

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof.

(v) anticonvulsants including for example carbamazepine, valproate, lamotrogine, gabapentin and equivalents and pharmaceutically active isomer(s) and metabolite(s) thereof.

(vi) Alzheimer's therapies including for example donepezil, memantine, tacrine and equivalents and pharmaceutically active isomer(s) and metabolite(s) thereof.

(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.

(viii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof.

(ix) stroke therapies including for example abciximab, activase, NXY-059, citicoline, crobenetine, desmoteplase, repinotan, traxoprodil and equivalents and pharmaceutically active isomer(s) and metabolite(s) thereof.

(x) urinary incontinence therapies including for example darafenacin, falvoxate, oxybutynin, propiverine, robalzotan, solifenacin, tolterodine and equivalents and pharmaceutically active isomer(s) and metabolite(s) thereof.

(xi) neuropathic pain therapies including for example gabapentin, lidoderm, pregablin and equivalents and pharmaceutically active isomer(s) and metabolite(s) thereof.

(xii) nociceptive pain therapies such as celecoxib, etoricoxib, lumiracoxib, rofecoxib, valdecoxib, diclofenac, loxoprofen, naproxen, paracetamol and equivalents and is pharmaceutically active isomer(s) and metabolite(s) thereof.

(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof.

(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in the publication reference. Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

### METHODS OF PREPARATION

The present invention also relates to processes for preparing the compound of formula (I) as a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in “Protective Groups in Organic Synthesis”, T. W. Greene, P. G. M Wutz, Wiley-Interscience, New York, 1999. It is understood that microwaves can be used for the heating of reaction mixtures.

Another aspect of the present invention provides a process for preparing a compound of formula (I), or a pharmaceutically acceptable salt thereof, wherein R¹³ and R¹⁴ are defined as A or B in formula (I) above; Rc is defined as for C in formula (I) above; and R¹ is, unless otherwise specified, as defined in formula (I). Said process comprises of:

### (i) Formation of a Corresponding Compound of Formula (V):

A compound of formula (V) may be obtained as depicted in Scheme 1, for example, by metallation or halogen metal exchange of a compound of formula (II), wherein G is either a hydrogen or a halogen respectively, to obtain an intermediate of formula (III), wherein L is a ligand such as halogen and n is between 0 and 6. The intermediate (III) is not isolated but reacted further with a compound of formula (IV), wherein LG is either N(CH₃)(OCH₃) or halogen or another suitable leaving group as for example described by R. K. Dieter, (Tetrahedron, 55 (1999) 4177-4236).

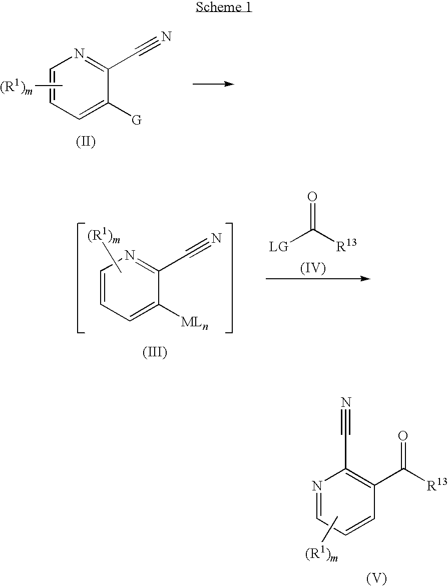

The reaction may be carried out by treating a compound of formula (II), wherein G is hydrogen or halogen (such as iodine or bromine), with an appropriate metallating reagent, such as a lithium reagent (such as tert-butyllithium, n-butyllithium, lithium diispropylamide or lithium tetramethyl piperidine) or with a Grignard reagent (such as isopropylmagnesium bromide) or with a metal (such as magnesium, zinc or manganese) by standard methods known in the art. Optionally, the formed intermediate of formula (III) may be further transmetallated by treating it with a metal salt or metal complex, such as copper cyanide or lithium bromide, to obtain a new intermediate of formula (III), and then treat said intermediate of formula (III) with a compound of formula (IV), wherein LG represents a leaving group such as a halogen, such as chlorine, or N(CH₃)(OCH₃). Optionally, this transformation may be performed under the influence of a transition metal catalyst such as a palladium salt or complex as for example described in literature (Tetrahedron, 55 (1999) 4177-4236). The reaction may be performed in a suitable solvent, such as diethyl ether or tetrahydrofuran, at a temperature between −105° C. and room temperature.

### (ii) Formation of a Corresponding Compound of Formula (VIII):

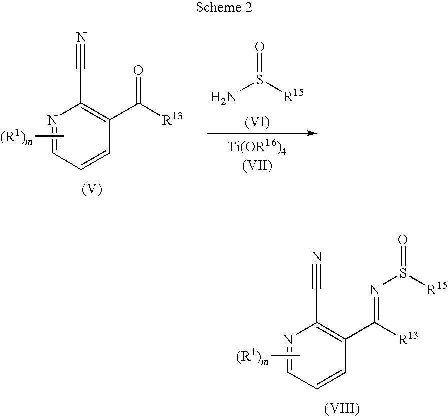

A compound of formula (VIII) may be obtained by reacting a compound of formula (V) with a compound of formula (VI) (Scheme 2), wherein R¹⁵ is alkyl (such as for example ten-butyl). The reaction is performed in the presence of a suitable Lewis acid of formula (VII), wherein R¹⁶ is alkyl (such as ethyl or isopropyl). The reaction is performed in a suitable solvent (such as diethyl ether or tetrahydrofuran) at a temperature between room temperature and reflux temperature

(iii) Formation of a Corresponding Compound of Formula (XI)

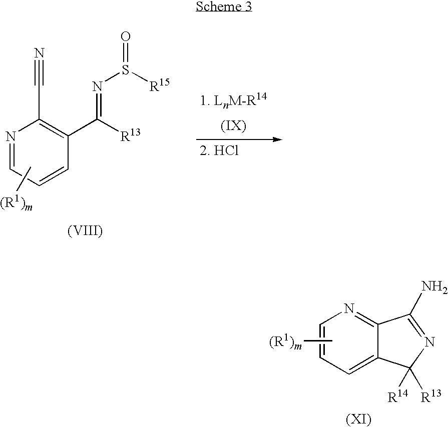

A compound of formula (XI) may be prepared by treating a compound of formula (VIII), with an appropriate organo metallic reagent of formula (IX), wherein M is a metal (such as lithium, magnesium or zinc), wherein L represents a ligand such as halogen and n is between 0 and 2, and wherein R¹⁴ is as defined above, followed by the treatment with a suitable acid, such as hydrochloric acid. The reaction is performed in a suitable solvent, such as diethyl ether or tetrahydrofuran, at a temperature between −105° C. and room temperature. The organo metallic reagent of formula (IX) may be generated from the corresponding LG-R¹⁴, wherein LG represents a leaving group such as a halogen (such as iodide, bromide or chlorine) by methods as described in, for example, Advanced Organic Chemistry by Jerry March 4^{th} edition, Wiley Interscience,

### (iv) Formation of a Corresponding Compound of Formula (XIV)

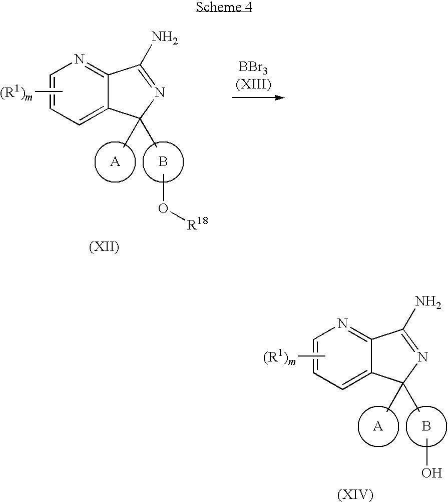

A compound of formula (XIV) can be obtained, as shown in Scheme 4, by reacting a compound of formula (XII), wherein R¹⁸ is defined as an alkyl (such as methyl or ethyl) with a reagent of formula (XIII), such as boron tribromide, in a suitable solvent (such as dichloromethane), at a temperature between 0° C. and room temperature.

### (v) Formation of a Corresponding Compound of Formula (XV)

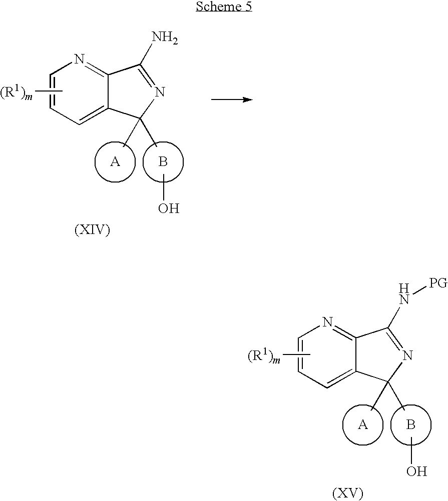

A compound of formula (XV), wherein PG is a suitable protecting group such as Boc, can be obtained, as shown in Scheme 5, by reacting a compound of formula (XIV) with a is suitable reagent (such as di-tert-butyl dicarbonate) mediated by a suitable base (such as 4-dimethylaminopyridine) in a suitable solvent (such as THF). A compound of formula (XV) may also be obtained with other protecting groups (PG) described in Protective Groups in Organic Synthesis by T. W. Greene, P. G. M Wutz, 3^{rd} Edition, Wiley-Interscience, New York, 1999.

### (vi) Formation of a Corresponding Compound of Formula (XVI)

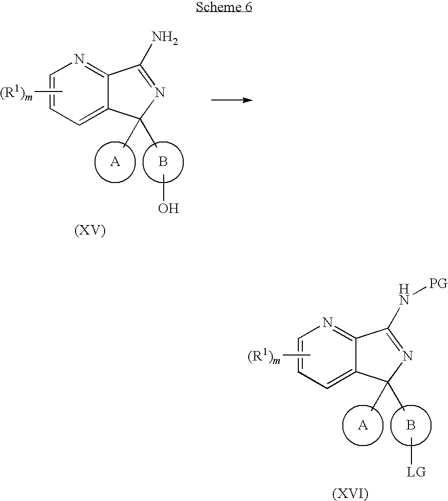

A compound of formula (XVI) can be obtained, wherein LG represents a suitable leaving group (such as an alkyl-, aryl- or haloalkyl-sulfonate (such as triflate)), as shown in Scheme 6, by reacting a compound of formula (XV), wherein PG is described above, with a suitable reagent (such as methansulfonyl chloride, trifluoromethanesulfonic anhydride or N-phenyltrifluoromethanesulphonimide), in the presence of a suitable base such as (N,N-diisopropylethylamine or potassium carbonate), in a suitable solvent (such as dichloromethane or THF), at a temperature range between 0 and 120° C.

(vii) Formation of a Corresponding Compound of Formula (I)

A compound of formula (I) may be obtained (Scheme 7) by starting from, for example, a compound of formula (XVI), wherein LG represents a leaving group such as halogen (such as chlorine, bromine or iodine) or an alkyl-, aryl- or haloalkyl-sulfonate (such as triflate), and reacting said compound of formula (XVI) with a compound of formula (XVII), wherein R^{C} is defined as above and T represents a boronic acid, a boronic ester or a stannane, in the presence of a transition metal catalyst as described, for example, in Metal Catalyzed Cross-coupling Reactions by F. Diederich and P. J. Stang, Wiley VCH, Weinheim, 1998. The compound of formula (XVII) may be generated from the corresponding LG-R^{C}, wherein LG represents a leaving group such as a halogen, (such as iodide, bromide or chlorine) or an alkyl-, aryl- or haloalkyl-sulfonate (such as triflate), by known methods as described in, for example, Advanced Organic Chemistry by Jerry March 4^{th} edition, Wiley Interscience,

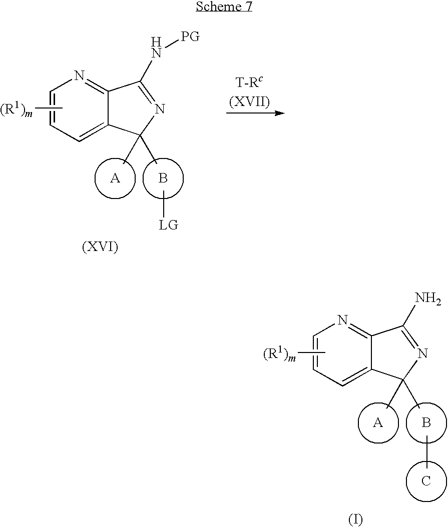

The reaction may be carried out using a suitable metal catalyst such as a palladium (such as [1,1′-bis(diphenylphosphino)ferrocene]palladium(II) chloride, tetrakis(triphenylphosphine)-palladium(0), palladium diphenylphosphineferrocene dichloride, palladium(II) acetate or bis(dibenzylideneacetone) palladium (0)). Optionally, a suitable ligand, such as triphenylphosphine, tri-tert-butylphosphine or 2-(dicyclohexylphosphino)biphenyl, or zinc and sodium triphenylphosphinetrimetasulfonate is used. A suitable base, such as cesium fluoride, an alkyl amine, such as triethyl amine, or an alkali metal or alkaline earth metal carbonate or hydroxide such as potassium carbonate, sodium carbonate, caesium carbonate, or sodium hydroxide, may be used in the reaction. Said reaction may be performed at a temperature range between +20° C. and +160° C., in a suitable solvent, such as toluene, tetrahydrofuran, dioxane, dimethoxyethane, water, ethanol, N,N-dimethylacetamide or N,N-dimethylformamide, or mixtures thereof.

Compounds of formula (II), (IV), (VI), (VII), (IX), (XIII), and (XVII) are commercially available compounds, or they are known in the literature, or they are prepared by standard processes known in the art.

### General Methods

All solvents used were of analytical grade and commercially available anhydrous solvents were routinely used for reactions.

Starting materials used were available from commercial sources, or prepared according to literature procedures.

Microwave heating was performed in a Creator, Initiator or Smith Synthesizer Single-mode microwave cavity producing continuous irradiation at 2450 MHz.

¹H NMR spectra were recorded in the indicated deuterated solvent at 400 MHz. The 400 MHz spectra were obtained unless stated otherwise, using a Bruker av400 NMR spectrometer equipped with a 3 mm flow injection SEI¹H/D-¹³C probe head with Z-gradients, using a BEST 215 liquid handler for sample injection, or using a Bruker DPX400 NMR spectrometer equipped with a 4-nucleus probehead with Z-gradients. Bruker 500 MHz Avance III NMR spectrometer, operating at 500 MHz for ¹H, 125 MHz for ¹³C, and 50 MHz for ¹⁵N equipped with a 5 mm TXI probehead with Z-gradients. Chemical shifts are given in ppm down- and upheld from TMS. Resonance multiplicities are denoted s, d, t, q, m and br for singlet, doublet, triplet, quartet, multiplet, and broad respectively.

LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm. Or LC-MS analyses were performed on a LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was configured with an atmospheric pressure chemical ionisation (APCI) ion source which was further equipped with atmospheric pressure photo ionisation (APPI) device. The mass spectrometer scanned in the positive mode, switching between APCI and APPI mode. The mass range was set to m/z 120-800 using a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode. Separation was performed using a Gemini column C18, 3.0 mm×50 mm, 3 μm, (Phenomenex) and run at a flow rate of 1 ml/min. A linear gradient was used starting at 100% A (A: 10 mM ammonium acetate in 5% methanol) and ending at 100% B (methanol). The column oven temperature was set to 40° C.

Mass spectra (MS) were run using an automated system with atmospheric pressure chemical (APCI or CI) or electrospray (+ESI) ionization. Generally, only spectra where parent masses are observed are reported. The lowest mass major ion is reported for molecules where isotope splitting results in multiple mass spectral peaks (for example when chlorine is present).

GC-MS analyses were performed on a Agilent 6890N GC equipped with a Chrompack CP-Sil 5CB column (25 m×0.25 mm i.d. df=0.25)), coupled to an Agilent 5973 Mass Selective Detector operating in a chemical ionization (CI) mode and the MS was scanned between m/z 50-500.

UPLCMS analyses were performed on an Waters Acquity UPLC system consisting of a Acquity Autosampler, Acquity Sample Organizer, Acquity Column Manager, Acquity Binary Solvent Manager, Acquity UPLC PDA detector and a Waters SQ Detector.

The mass spectrometer was equipped with an electrospray ion source (ES) operated in positive and negative ion mode. The capillary voltage was set to 3.0 kV and the cone voltage to 30 V, respectively. The mass spectrometer was scanned between m/z 100-600 with a scan time of 0.105 s. The diode array detector scanned from 200-400 nm. The temperature of the Column Manager was set to 60° C. Separation was performed on a Acquity column, UPLC BEH, C18 1.7 μM run at a flow rate of 0.5 ml/min. A linear gradient was applied starting at 100% A (A: 10 mM NH₄OAc in 5% CH3CN) ending at 100% B (B: CH3CN) after 1.3 min then 100% B for 0.6 min.

Acquity column, UPLC BEH, C18 1.7 μM. Linear gradient, flow 0.5 ml/min.

0-100% B (MeCN) in 1.3 min, then 100% B for 0.6 min. ESpos/ESneg, m/z 100-600. A (A: 10 mM NH₄OAc in 5% CH3CN)

Acquity column, UPLC BEH, C18 1.7 μM. Linear gradient, flow 0.5 ml/min, 0-100% B (MeCN) in 2.5 min, then 100% B until 3.8 min. ES+/ES−, m/z 100-600.

A (A: 10 mM NH₄OAc in 5% CH3CN)

GC-MS analyses were performed on a Agilent 6890N GC equipped with a Chrompack CP-Sil 5CB column (25 m×0.25 mm i.d. df=0.25)), coupled to an Agilent 5973 Mass Selective Detector operating in a chemical ionization (CI) mode and the MS was scanned between m/z 50-500.

Accurate mass analyses were performed on a QTOF micro (Waters). The mass spectrometer was equipped with an electrospray ionsource that uses two probes, a sample probe and a lock mass probe, respectively. The lock mass solution was Leucine Enkephaline (0.5 ng/μL in MilliQ water) infused at flow rate of 0.1 mL/min. The reference scan frequency was set to 5.5 s. Before the analysis, the mass spectrometer was calibrated in the positive mode between 90-1000 Da using a solution of NaFormate. The mass spectrometer scanned in the centroid mode between m/z 100-1000 with a scan time of 1.0 s. The capillary voltage was set to 3.3 kV and the ES cone voltage was set to 28 V. The source temperature and desolvation temperature were set to 110° C. and 350° C., respectively. The collision energy was set to 6.0 V. The QTOF micro was equipped with an LC(HP 1100 Agilent, Degasser, Binary pump, ALS and a column compartment). The column used was a Gemini C18, 3.0×50 mm, 3 u run at a flowrate of 1.0 mL/min. A linear gradient was applied starting at 100% A (A: 10 mM ammonium acetate) and ending at 100% B (B: acetonitrile) after 4 min. The column oven temperature was set to 40° C. The flow was split 1:4 prior to the ion source. 3 μL of the sample was injected on the column.

HPLC assays were performed using an Agilent HP 1100 Series system equipped with a Waters X-Terra MS, C₈ column (3.0×100 mm, 3.5 μm). The column temperature was set to 40° C. and the flow rate to 1.0 mL/min. The Diode Array Detector was scanned from 200-300 nm. A linear gradient was applied, run from 0% to 100% B in 4 min. Mobile phase A: 10 mM ammonium acetate in water/acetonitrile (95:5), mobile phase B: acetonitrile.

Preparative HPLC was performed on a Waters Auto purification HPLC-UV system with a diode array detector using a Waters XTerra MS C₈ column (19×300 mm, 7 μm) and a linear gradient of mobile phase B was applied. Mobile phase A: 0.1 M ammonium acetate in water/acetonitrile (95:5) and mobile phase B: acetonitrile. Flow rate: 20 mL/min. Thin layer chromatography (TLC) was performed on Merck TLC-plates (Silica gel 60 F₂₅₄) and spots were UV visualized. Flash chromatography was performed using Merck Silica gel 60 (0.040-0.063 mm), or employing a Combi Flash® Companion™ system using RediSep™ normal-phase flash columns.

Room temperature refers to 20-25° C.

Solvent mixture compositions are given as volume percentages or volume ratios.

### Terms and Abbreviations:

atm: atmospheric pressure;
Boc: t-butoxycarbonyl;
Cbz: benzyloxycarbonyl;
DAST: (diethylamino)sulphur trifluoride
DCM: dichloromethane;
DIPEA: diisopropylethylamine;
DMF: N;N-dimethyl formamide;
DMSO: dimethyl sulfoxide;
Et₂O: diethyl ether;
EtOAc: ethyl acetate;
h: hour(s);
HPLC: high pressure liquid chromatography;
min: minute(s);
MeOH: methanol;
NMR: nuclear magnetic resonance;
psi: pounds per square inch;
TFA: trifluoroacetic acid;
THF: tetrahydrofuran;
ACN: acetonitrile.
r.t. room temperature
sat saturated
aq aqueous

Compounds have been named using CambridgeSoft MedChem ELN v2.1 or ACD/Name, to version 9.0, software from Advanced Chemistry Development, Inc. (ACD/Labs), Toronto ON, Canada, www.acdlabs.com, 2004.

### EXAMPLES

Below follows a number of non-limiting examples of compounds of the invention.

### Example 1i

### 3-(3-methoxybenzoyl)picolinonitrile

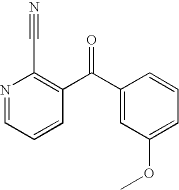

3-Bromopicolinonitrile (2.8 g, 15.30 mmol) in dry THF (50 mL) was added dropwise over 1.5 h to a bottle of Rieke(R) Zinc (50.0 mL, 38.25 mmol) under N₂ and stirred for 1 h at r.t. The reaction mixture was cooled to −20° C. and stirred for 22 h. The excess Zn was removed by decantation, and the solution was cooled to −20° C. CuCN (LiBr)₂ (in THF 1M) (15.30 mL, 15.30 mmol) was added to the solution. The reaction mixture was allowed to reach 0° C. and stirred for 30 min. The mixture was cooled to −40° C. and 3-methoxybenzoyl chloride (2.26 mL, 16.1 mmol) was added. The reaction mixture was allowed to reach r.t. over night. Aqueous NH₄Cl (sat.) was added and the mixture was extracted with EtOAc. The organic phase was washed with NaHCO₃ (sat.) and brine, dried over MgSO₄ and concentrated. Chromatography using 0-40% EtOAc in n-heptane gave (2.2 g, 60% yield) of the title compound:

¹H NMR (500 MHz, DMSO-d₆) d ppm 8.94-8.97 (m, 1H), 8.20-8.24 (m, 1H), 7.87-7.91 (m, 1H), 7.50-7.54 (m, 1H), 7.32-7.38 (m, 3H), 3.83 (s, 3H).

### Example 2i

### 3-(3-bromobenzoyl)picolinonitrile

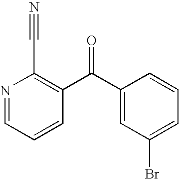

The title compound was synthesized as described for Example 11 in 46% yield starting from 3-bromopicolinonitrile (2.9 g, 15.85 mmol) and 3-bromobenzoyl chloride (2.087 mL, 15.85 mmol).

¹H NMR (500 MHz, DMSO-d₆) δ ppm 8.95-8.99 (m, 1H) 8.22-8.26 (m, 1H) 7.96-8.00 (m, 2H) 7.88-7.92 (m, 1H) 7.79-7.83 (m, 1H) 7.55-7.59 (m, 1H).

### Example 3i

### N-((2-cyanopyridin-3-yl)(3-methoxyphenyl)methylene)-2-methylpropane-2-sulfinamide

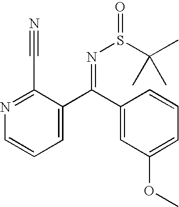

2-Methyl-2-propanesulfinamide (1.824 g, 15.05 mmol) was added to a mixture of titanium(IV) ethoxide (7.17 mL, 34.21 mmol) and 3-(3-methoxybenzoyl)picolinonitrile (3.26 g, 13.68 mmol) in THF (60 mL). The reaction mixture was heated to reflux and stirred for 42 h. MeOH (7 mL), NaHCO₃ (sat, 7 drops) and EtOAc was added and the slurry was filtered through celite and MgSO₄ and then concentrated. Column chromatography using 0-45% EtOAc in heptane gave (3.22 g, 69% yield) of the title compound.

¹H NMR (500 MHz, DMSO-d₆) δ ppm 8.78-8.84 (m, 1H), 7.97-8.22 (m, 1H), 7.76-7.88 (m, 1H), 7.42 (t, 1H), 7.19-7.25 (m, 1H), 7.10-7.14 (m, 1H), 6.94-7.00 (m, 1H), 3.77 (s, 3H), 1.23-1.30 (m, 9H). MS (ES+) m/z 342 [M+1]⁺.

### Example 4i

### N-((3-Bromophenyl)(2-cyanopyridin-3-yl)methylene)-2-methylpropane-2-sulfinamide

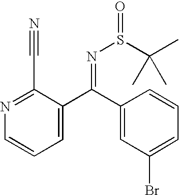

The title compound was synthesized as described for Example 3i in 57% yield starting from 3-(3-bromobenzoyl)picolinonitrile (2.11 g, 7.35 mmol) and 2-methyl-2-propanesulfinamide (1.158 g, 9.55 mmol). ¹H NMR (500 MHz, DMSO-d₆) δ ppm 8.82-8.88 (m, 1H) 8.03-8.30 (m, 1H) 7.80-7.91 (m, 2H) 7.74 (s, 1H) 7.48-7.52 (m, 2H) 1.29 (br. s., 9H).

### Example 5i

### 5-(2,6-Dimethylpyridin-4-yl)-5-(3-methoxyphenyl)-5H-pyrrolo[3,4-b]pyridin-7-amine

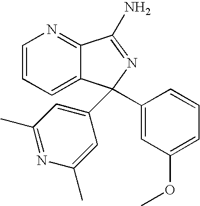

N-((2-cyanopyridin-3-yl)(3-methoxyphenyl)methylene)-2-methylpropane-2-sulfinamide (1.3 g, 3.81 mmol) in THF (8 mL) was added to a mixture of t-BuLi (5.71 mL, 9.14 mmol) and 4-bromo-2,6-dimethylpyridine (0.815 g, 4.38 mmol) in THF (24 mL), at −70° C. The reaction mixture was stirred at −70° C. for 1 h where after the mixture was allowed to reach r.t. Water, NaHCO₃ and EtOAc was added, the organic phase was collected, dried over MgSO₄ and concentrated. The residue was dissolved in methanol (20 mL) and treated with HCl (2M in diethyl ether) (1.904 mL, 3.81 mmol) for 4 h. Water and NH₄OH (conc.) was added, and the mixture was extracted with DCM, the organic phase was dried over MgSO₄ and concentrated. Column chromatography using 0-3% MeOH(NH₃) in DCM gave (0.65 g, 50% yield) of the title compound:

¹H NMR (500 MHz, DMSO-d₆) δ ppm 8.61-8.65 (m, 1H), 8.27-8.32 (m, 1H), 7.44-7.50 (m, 1H), 7.20 (t, 1H), 6.97 (s, 2H), 6.85-6.90 (m, 2H), 6.77-6.85 (m, 3H), 3.67 (s, 3H), 2.34 (s, 6H);

### Example 6i

### 5-(3-Bromophenyl)-5-(2-(trifluoromethyl)pyridin-4-yl)-5H-pyrrolo[3,4-b]pyridin-7-amine

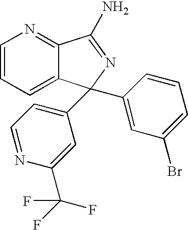

The title compound was synthesized as described for Example 5i in 21% yield starting from (E)-N-((3-bromophenyl)(2-cyanopyridin-3-yl)methylene)-2-methylpropane-2-sulfinamide (810 mg, 2.08 mmol) and 4-bromo-2-(trifluoromethyl)pyridine (586 mg, 2.59 mmol):

¹H NMR (500 MHz, DMSO-d₆) δ ppm 8.68-8.73 (m, 2H), 8.47-8.51 (m, 1H), 7.68-7.75 (m, 2H), 7.52-7.58 (m, 1H), 7.47-7.51 (m, 2H), 7.37-7.41 (m, 1H), 7.30 (t, 1H), 7.14 (br. s., 2H); MS (ES) m/z 433, 435 [M+1]⁺.

### Example 7i

### 3-(7-Amino-5-(2,6-dimethylpyridin-4-yl)-5H-pyrrolo[3,4-b]pyridin-5-yl)phenol

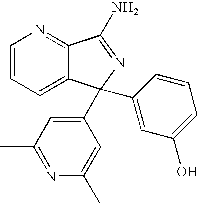

5-(2,6-dimethylpyridin-4-yl)-5-(3-methoxyphenyl)-5H-pyrrolo[3,4-b]pyridin-7-amine (0.65 g, 1.89 mmol) was dissolved in DCM (30 mL) and cooled to 0° C. Boron tribromide (0.55 mL, 5.66 mmol) was added and the reaction mixture was stirred at 0° C. for 2 h, the mixture was allowed to reach to rt and stirring was continued for 4 h. NH₄OH(conc) (8 mL) and MeOH (15 mL) was added and pH was adjusted to ˜7-8 using HCl (2M) and NH₄OH (conc). The mixture was extracted with EtOAc and the organic phase was dried over MgSO₄, filtered and concentrated, to afford the title compound 0.62 g, (99% yield). The title compound was used in next step without further purification. MS (ES+) m/z 303 [M+1]⁺.

### Example 8i

### tert-Butyl 5-(2,6-dimethylpyridin-4-yl)-5-(3-hydroxyphenyl)-5H-pyrrolo[3,4-b]pyridin-7-ylcarbamate

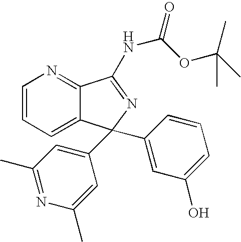

Di-tert-butyl dicarbonate (0.901 g, 4.13 mmol) was added to a mixture of 3-(7-amino-5-(2,6-dimethylpyridin-4-yl)-5H-pyrrolo[3,4-b]pyridin-5-yl)phenol (0.62 g, 1.88 mmol) and 4-dimethylaminopyridine (0.023 g, 0.19 mmol) in THF (25 mL). The mixture was stirred over night at 40° C. Brine and water was added and the mixture and extracted with EtOAc. The organic phase was dried over MgSO₄ and concentrated. The residue was dissolved in MeOH (25 mL) and ammonia (conc.) (10 mL), the mixture was heated to 45° C. and stirred for 4 h. The mixture was cooled to rt, concentrated and NH₄Cl (sat.) was added. The phases were separated and the aqueous phase was extracted with EtOAc. The combined organic phases were dried over MgSO₄ and concentrated. Column chromatography 10-90% EtOAc in heptane gave the title compound (0.45 g, 56% yield):

¹H NMR (500 MHz, DMSO-d₆) δ ppm 9.66-9.76 (m, 1H), 9.38-9.46 (m, 1H), 8.65-8.73 (m, 1H), 8.28-8.39 (m, 1H), 7.49-7.59 (m, 1H), 7.05-7.18 (m, 1H), 6.98-7.04 (m, 2H), 6.57-6.75 (m, 3H), 2.34-2.40 (m, 6H), 1.46-1.52 (m, 9H); MS (ES+) m/z 431 [M+1]⁺.

### Example 9i

### 3-(7-(tert-Butoxycarbonylamino)-5-(2,6-dimethylpyridin-4-yl)-5H-pyrrolo[3,4-b]pyridin-5-yl)phenyl trifluoromethanesulfonate

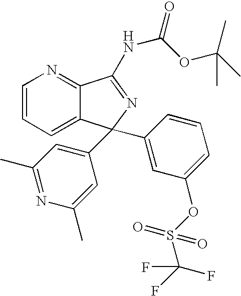

Trifluoromethanesulfonic anhydride (0.164 mL, 0.98 mmol) was added to tert-butyl 5-(2,6-dimethylpyridin-4-yl)-5-(3-hydroxyphenyl)-5H-pyrrolo[3,4-b]pyridin-7-ylcarbamate (0.35 g, 0.81 mmol) and N,N-diisopropylethylamine (0.425 mL, 2.44 mmol) in DCM (15 mL) and the mixture was stirred over night. Water was added and the mixture was extracted with DCM. The organic phases were washed with brine, dried over MgSO₄ and concentrated to afford the title compound in quantitative yield. The title compound was used in the next step without further purification. MS (ES) m/z 563 [M+1]⁺.

### Example 10i

### 3-(3-Bromo-4-fluoro-benzoyl)-pyridine-2-carbonitrile

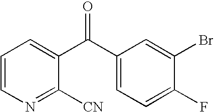

3-Bromopicolinonitrile (2.4 g, 13.11 mmol) was dissolved in dry THF (20 mL) and added dropwise over 1.5 hours to a bottle of Rieke® Zinc (5.0 g in 100 mL of THF, 40.98 mmol) under a nitrogen atmosphere. The resulting mixture was stirred for 1 hour at room temperature (conversion to the zincate was checked by quenching with D₂O) and then left at −20° C. overnight. The solution was then carefully decanted to remove excess of zinc and cooled to −20° C. A freshly prepared solution of CuCN(LiBr)₂ complex in dry THF (1M, 22.95 mL, 22.95 mmol) was added slowly to the above solution and the reaction mixture was allowed to reach 0° C. and stirred for 30 minutes. The mixture was then cooled to ±40° C. and 3-bromo-4-fluoro-benzoyl chloride (3.1 g, 13.08 mmol) was added dropwise over 5 minutes. The reaction mixture was warmed to room temperature, stirred overnight, quenched with saturated NH₄Cl solution and extracted with ethyl acetate (3×100 mL). The combined extracts were washed with saturated NaHCO₃ solution (2×50 mL), dried over MgSO₄ and concentrated under reduced pressure. The residue was triturated with hexane/Et₂O to afford 3.27 g (82% yield) of 3-(3-bromo-4-fluoro-benzoyl)-pyridine-2-carbonitrile that was used in the next step without any purification.

### Example 11i

### 2-Methyl-propane-2-sulfinic acid (3-bromo-4-fluoro-phenyl)-(2-cyano-pyridin-3-yl)-methyleneamide

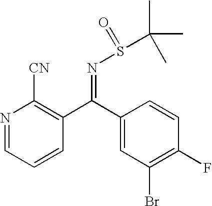

3-(3-Bromo-4-fluoro-benzoyl)-pyridine-2-carbonitrile (3 g, 9.83 mmol) followed by 2-methyl-2-propanesulfinamide (1.9 g, 15.67 mmol) were added to a solution of titanium(IV) ethoxide (5.1 mL, 24.58 mmol) in dry THF (200 mL). The reaction mixture was refluxed for 48 hours, cooled to room temperature and quenched with MeOH (5 mL) followed by saturated NaHCO₃ solution (7 drops). The resulting suspension was stirred for 30 minutes, EtOAc (25 mL) was added and the slurry was filtered through a pad of Celite and MgSO₄. The filtrate was concentrated in vacuo and the residue was purified by flash chromatography using a gradient from 0-20% EtOAc in hexane to afford 3.2 g (80% yield) of the title compound.

¹H NMR (400 MHz, CDCl₃) δ ppm 8.74 (br. s., 1H), 7.75 (br. s., 1H), 7.68 (d, 1H), 7.55 (dd, 1H), 7.39 (br. s., 1H), 7.08-7.14 (m, 1H), 1.31 (s., 9H); MS (ES+) m/z: 409.95 [M+1]⁺.

### Example 12i

### 5-(3-Bromo-4-fluorophenyl)-5-(3,5-difluoro-4-methoxyphenyl)-5H-pyrrolo[3,4-b]pyridin-7-amine

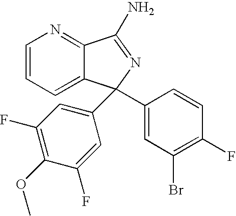

tert-Butyllithium (0.995 mL, 1.59 mmol) was added dropwise to THF (4 mL) at −100° C. under an argon atmosphere. A solution of 1,3-difluoro-5-iodo-2-methoxybenzene (215 mg, 0.80 mmol) in THF (1 mL) was added dropwise followed by the addition of N-((3-bromo-4-fluorophenyl)(2-cyanopyridin-3-yl)methylene)-2-methylpropane-2-sulfinamide (250 mg, 0.61 mmol) in THF (2 mL). The resulting reaction mixture was left on the thawing cooling bath for 30 min then the cooling bath was removed and the mixture was stirred at r.t. for 1 h. Hydrogen chloride in methanol (3 mL, 3.7 mmol) was added and the resulting mixture was stirred at r.t. for 1 h. The mixture was concentrated and purified on a silica gel column eluted with 0-10% NH₃ (0.1 M in MeOH) in DCM. This gave 52 mg (19% yield) of the title product:

MS (ES) m/z 448, 450 [M+1]⁺.

### Example 13i

### 5-(3-Bromophenyl)-5-(3-chloro-4-methoxyphenyl)-5H-pyrrolo[3,4-b]pyridin-7-amine

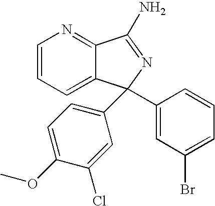

n-Butyllithium (0.750 mL, 1.20 mmol) was added to a solution of 4-bromo-2-chloro-1-methoxybenzene (244 mg, 1.10 mmol) in THF (1.5 mL) at −78° C. under an argon atmosphere. The mixture was stirred for 5 min, then a solution of N-((3-bromophenyl)(2-cyanopyridin-3-yl)methylene)-2-methylpropane-2-sulfinamide (390 mg, 1 mmol) in THF (1.5 mL) was added. The resulting mixture was stirred at −78° C. for 15 min, then the cooling bath was removed and the mixture was stirred at rt for 1.5 h. Hydrogen chloride in methanol (3 mL, 3.75 mmol) was added and the mixture was stirred at rt for 1 h. Saturated aqueous NaHCO₃ (3 mL) was added followed by DCM (3 mL). The mixture was poured into a phase separator and the organic phase was collected, concentrated and purified on a silica gel column eluted with 0-5% 0.1M NH₃ in MeOH in DCM to afford 355 mg (83% yield) of the title compound:

¹H NMR (500 MHz, DMSO-d₆) δ ppm 8.65 (d, 1H) 8.33 (dd, 1H) 7.49 (dd, 1H) 7.41-7.47 (m, 2H) 7.29-7.36 (m, 2H) 7.22-7.29 (m, 2H) 7.06 (d, 1H) 6.90 (br. s., 2H) 3.81 (s, 3H); MS (ES+) m/z 428, 430 [M+1]⁺.

### Example 14i

### 4-Bromo-2-cyclopropyl-1-(difluoromethoxy)benzene

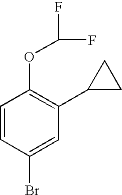

A three-necked round bottom flask (500 mL) equipped with an acetone/dry ice condenser (−78° C.) was charged with a solution of 4-bromo-2-cyclopropyl-phenol (9.0 g, 42.25 mmol) in iPrOH (100 mL). Aqueous sodium hydroxide solution (20%, 100 mL) was added. The reaction mixture was stirred vigorously at 40° C. for 5 hours while chlorodifluoromethane was bubbled continuously into the solution at a moderate rate. The reaction mixture was then cooled to room temperature and extracted with Et₂O (2×50 mL). The combined extracts were washed with water (30 mL), dried over magnesium sulfate and concentrated in vacuo. Purification of the crude mixture by flash column chromatography using pentane afforded 6.0 g (54% yield) of 4-bromo-2-cyclopropyl-1-difluoromethoxy-benzene after careful condensation at lower temperature (to avoid possible loss of the material):

¹H NMR (400 MHz, CDCl₃) δ ppm 7.26 (dd, 1H), 6.99 (d, 1H), 6.97 (d, 1H), 6.69 (t, 1 H), 2.13 (tt, 1H), 0.95-1.07 (m, 2H), 0.62-0.72 (m, 2H); ¹⁹F NMR (376 MHz, CDCl₃) δ ppm-80.54.

### Example 15i

### 5-(3-Bromophenyl)-5-(3-cyclopropyl-4-(difluoromethoxy)phenyl)-5H-pyrrolo[3,4-b]pyridin-7-amine

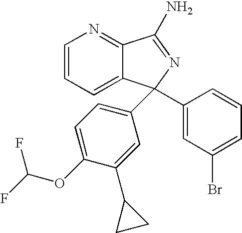

The title compound was synthesized as described for Example 13i in 68% yield starting from N-((3-bromophenyl)(2-cyanopyridin-3-yl)methylene)-2-methylpropane-2-sulfinamide (195 mg, 0.5 mmol) and 4-bromo-2-cyclopropyl-1-(difluoromethoxy)benzene (145 mg, 0.55 mmol):

MS (ES+) m/z 470, 472 [M+1]⁺.

### Example 16i

### 3-Bromo-5-chloropicolinonitrile

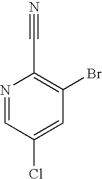

2,3-Dibromo-5-chloropyridine (14 g, 51.6 mmol), copper(I) cyanide (5.09 g, 56.79 mmol) and propionitrile (58 mL) were divided into four vials and each vial was heated in a microwave reactor at 150° C. for 2.5 h. The mixtures were pooled, filtered and concentrated. The resulting residue was taken up in DCM (100 mL), a solid was filtered off and the filtrate was concentrated to afford 11.3 g (quantitative yield) of the title compound:

MS (CI) m/z 217, 219 [M+H]⁺.

### Example 17i

### 3-(3-Bromobenzoyl)-5-chloropicolinonitrile

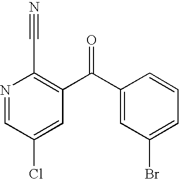

The title compound was synthesized as described for Example 11 in 51% yield starting from 3-bromo-5-chloropicolinonitrile (11.09 g, 51 mmol) and 3-bromobenzoyl chloride (6.74 mL, 51.00 mmol):

MS (CI) m/z 321, 323 [M+H]⁺.

### Example 18i

### N-((3-Bromophenyl)(5-chloro-2-cyanopyridin-3-yl)methylene)-2-methylpropane-2-sulfinamide

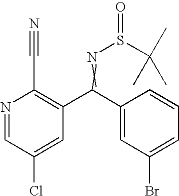

The title compound was synthesized as described for Example 3i in 57% yield starting from 3-(3-bromobenzoyl)-5-chloropicolinonitrile (8.33 g, 25.91 mmol) and 2-methyl-2-propanesulfinamide (3.77 g, 31.09 mmol):

MS (ES+) m/z 424, 426 [M+1]⁺.

### Example 19i

### 5-(3-Bromophenyl)-3-chloro-5-(2-methylpyridin-4-yl)-5H-pyrrolo[3,4-b]pyridin-7-amine

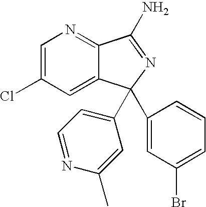

The title compound was synthesized as described for Example 5i in 54% yield starting from N-((3-bromophenyl)(5-chloro-2-cyanopyridin-3-yl)methylene)-2-methylpropane-2-sulfinamide (1 g, 2.35 mmol) and 4-bromo-2-methylpyridine (0.486 g, 2.83 mmol):

MS (ES+) m/z 413, 415 [M+1]⁺.

### Example 20i

### 3-Bromo-5-methylpicolinonitrile

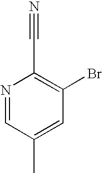

Potassium cyanide (5.76 g, 88.42 mmol) was added to a solution of 3-bromo-2-fluoro-5-methylpyridine (14 g, 73.68 mmol) in DMSO (75 mL) at rt. The resulting mixture was stirred at 110° C. for 1 h. More potassium cyanide (1.5 g, 23.03 mmol) was added and stirring continued for 20 min. Then the temperature was lowered to 80° C. and the mixture stirred over night. When cooled to rt, the mixture was poured into water (200 mL) and extracted with DCM (3×100 mL). The combined organics were washed with water (100 mL) then poured into a phase separator. The organic phase was collected, silica was added and the mixture was concentrated until a free flowing powder was obtained. The residue was purified on a silica gel column eluted with 0-50% EtOAc in heptane to afford 6.92 g (48% yield) of the title compound:

¹H NMR (400 MHz, DMSO-d₆) δ ppm 8.57-8.68 (m, 1H) 8.21-8.34 (m, 1H) 2.40 (s, 3 H); MS (CI) m/z 197, 199 [M+H]⁺.

### Example 21i

### 3-(3-Bromobenzoyl)-5-methylpicolinonitrile

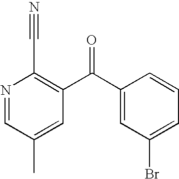

The title compound was synthesized as described for Example 11 in 66% yield starting from 3-bromo-5-methylpicolinonitrile (6.9 g, 35.02 mmol) and 3-bromobenzoyl chloride (5.09 mL, 38.52 mmol):

¹H NMR (400 MHz, DMSO-d₆) δ ppm 8.79-8.86 (m, 1H) 8.05-8.10 (m, 1H) 7.95-8.01 (m, 2H) 7.77-7.83 (m, 1H) 7.53-7.61 (m, 1H) 2.46 (s, 3H); MS (CI) m/z 301, 303 [M+H]⁺.

### Example 22i

### N-((3-Bromophenyl)(2-cyano-5-methylpyridin-3-yl)methylene)-2-methylpropane-2-sulfinamide

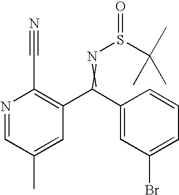

The title compound was synthesized as described for Example 3i in 76% yield starting from 3-(3-bromobenzoyl)-5-methylpicolinonitrile (6.98 g, 23.18 mmol) and 2-methyl-2-propanesulfinamide (3.37 g, 27.81 mmol):

¹H NMR (400 MHz, DMSO-d₆) δ ppm 8.69 (d, 1H) 7.90-8.11 (m, 1H) 7.82-7.89 (m, 1 H) 7.73 (s, 1H) 7.46-7.54 (m, 2H) 2.45 (s, 3H) 1.26 (s, 9H); MS (ES+) m/z 404, 406 [M+1]⁺.

### Example 23i

### 5-(3-Bromophenyl)-5-(4-methoxyphenyl)-3-methyl-5H-pyrrolo[3,4-b]pyridin-7-amine

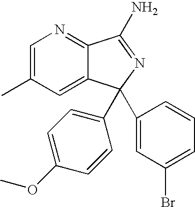

The title compound was synthesized as described for Example 13i in 93% yield starting from N-((3-bromophenyl)(2-cyano-5-methylpyridin-3-yl)methylene)-2-methylpropane-2-sulfinamide (2.022 g, 5 mmol) and 4-bromoanisole (0.689 mL, 5.50 mmol):

MS (ES+) m/z 408, 410 [M+1]⁺.

### Example 24i

### 5-Bromo-2-difluoromethoxy-1,3-dimethyl-benzene

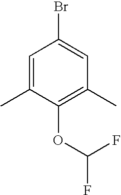

A three-necked round bottom flask (500 mL) equipped with a dry ice condenser (−78° C., acetone/dry ice) was charged with a solution of 4-bromo-2,6-dimethyl-phenol (12.0 g, 59.7 mmol) in iPrOH (100 mL) and aqueous sodium hydroxide solution (20%, 100 mL) was added. The reaction mixture was stirred vigorously at 40° C. for 5 hours while chlorodifluoromethane was bubbled continuously into the solution at a moderate rate. The reaction mixture was then cooled to room temperature and extracted with Et₂O (3×50 mL). The combined extracts were washed with water (2×30 mL), dried over magnesium sulfate and concentrated in vacuo. Purification of the crude mixture by flash column chromatography using pentane followed by recrystallization from MeOH afforded 12.6 g (84% yield) of the title compound:

¹H NMR (400 MHz, CDCl₃) δ ppm 7.26 (s, 1H), 7.21 (s, 1H), 6.50 (t, 1H), 2.28 (s, 3H); ¹⁹F NMR (376 MHz, CDCl₃) δ ppm-79.16 (d, J=75.9 Hz); CHN: Calcd for C₉H₉BrF₂O: C, 43.05, H, 3.61. Found: C, 42.72, H, 3.60.

### Example 25i

### 5-(3-Bromophenyl)-5-(4-(difluoromethoxy)-3,5-dimethylphenyl)-5H-pyrrolo[3,4-b]pyridin-7-amine

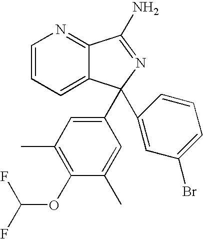

n-Butyllithium, 2.5 M in hexanes, (2.214 mL, 5.53 mmol) was added to isopropylmagnesium bromide, 1 M in THF, (2.77 mL, 2.77 mmol) in THF (32 mL) at 0° C. under argon atmosphere. The reaction mixture was stirred for 14 min, then cooled to −78° C. 5-Bromo-2-(difluoromethoxy)-1,3-dimethylbenzene (1.303 g, 5.19 mmol) in THF (11 mL) was added dropwise over 7 min. The reaction mixture was stirred for 1 h., and then N-((3-bromophenyl)(2-cyanopyridin-3-yl)methylene)-2-methylpropane-2-sulfinamide (1.35 g, 3.46 mmol) in THF (11 mL) was added dropwise over 8 min. The mixture was stirred at −78° C. for 1 h. The reaction was quenched with NH₄Cl (aq sat), diluted with water and extracted with EtOAc (×3), dried (Na₂SO₄), filtered and concentrated. Purification twice by silica gel flash chromatography, with eluents heptane/EtOAc 1:1-1:2 and CHCl₃/MeOH 50:1 gave the title compound (0.235 g, 14.8% yield):

¹H NMR (600 MHz, DMSO-d₆) δ ppm 8.64 (d, 1H), 8.34 (d, 1H), 7.45-7.53 (m, 2H), 7.43 (d, 1H), 7.34 (d, 1H), 7.25 (t, 1H), 7.12 (s, 2H), 6.90 (t, 1H), 6.83 (br. s., 2H), 2.17 (s, 6H); MS (ES+) m/z 458, 460 [M+1]⁺.

### Example 26i

### 5-(3-Bromophenyl)-5-(4-fluoro-3,5-dimethylphenyl)-5H-pyrrolo[3,4-b]pyridin-7-amine

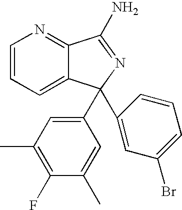

n-Butyllithium, 2.5 M in hexanes, (2.460 mL, 6.15 mmol) was added to isopropylmagnesium bromide, 1 M in THF, (3.07 mL, 3.07 mmol) in THF (36 mL) at 0° C. under argon atmosphere. The reaction was stirred for 13 min, then cooled to −78° C. 5-Bromo-2-fluoro-1,3-dimethylbenzene (0.807 mL, 5.76 mmol) in THF (12 mL) was added dropwise over 8 min. The reaction mixture was stirred for 30 min and then N-((3-bromophenyl)(2-cyanopyridin-3-yl)methylene)-2-methylpropane-2-sulfinamide (1.5 g, 3.84 mmol) in THF (12 mL) was added dropwise over 8 min. The mixture was stirred at −78° C. for 80 min and then most of the dry ice was removed from the cooling bath, and it was left to reach r.t. over night. HCl (0.5 M in MeOH) (30.7 mL, 15.37 mmol) was added and the reaction was stirred at r.t. for 5.5 h. The mixture was concentrated in vacuo, partitioned between NaHCO₃ (aq sat) and dichloromethane (×3), dried (Na₂SO₄), filtered and concentrated. A second reaction was performed as above starting with N-((3-bromophenyl)(2-cyanopyridin-3-yl)methylene)-2-methylpropane-2-sulfinamide (100 mg, 0.26 mmol). The two batches were pooled and then purified twice by silica gel flash chromatography, using as eluent CHCl₃/MeOH 20:1-10:1 and heptane/EtOAc 1:2 to give the title compound (74 mg, 10% yield):

¹H NMR (400 MHz, DMSO-d₆) δ ppm 8.63 (dd, 1H), 8.30 (dd, 1H), 7.47 (dd, 1H), 7.38-7.45 (m, 2H), 7.29-7.34 (m, 1H), 7.24 (t, 1H), 7.06 (d, 2H), 6.84 (br. s., 2H), 2.14 (d, 6H); MS (ES+) m/z 410/412 [M+1]⁺.

### Example 27i

### 2-Fluoro-6-methylphenol

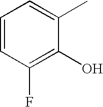

3-Fluoro-2-hydroxybenzaldehyde (2.5 g, 17.84 mmol) was dissolved in methanol (200 mL). Pd/C 10% (0.25 g, 2.35 mmol) was added under a stream of nitrogen. The mixture was hydrogenated at 50 psi and 50° C. for 16 h. Pd/C 10% (0.25 g, 2.35 mmol) and hydrochloric acid (conc, 2 ml) were added and the mixture was hydrogenated at 50 psi and 50° C. for 5 h. The mixture was filtered through a pad of diatomeous earth and the filter was washed with methanol. The mixture was concentrated to ca 5 mL. The residue was partitioned between brine and diethyl ether. The aqueous phase was extracted with dichloromethane. The combined organic phases were dried (MgSO₄) and evaporated to give 2-fluoro-6-methylphenol (0.950 g, 42% yield):

¹H NMR (500 MHz, DMSO-d₆) δ ppm 9.29 (br. s., 1H) 6.92 (m, 2H) 6.69 (m, 1H) 2.16 (s, 3H); MS (EI+) m/z 126 [M+].

### Example 28i

### 4-Bromo-2-fluoro-6-methylphenol

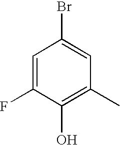

2-Fluoro-6-methylphenol (0.95 g, 7.53 mmol) was dissolved in acetic acid (15 mL). The mixture was cooled on an ice-water bath. N-bromosuccinimide (1.41 g, 7.91 mmol) was added portion wise and the mixture was let to RT and was stirred at RT for 3 h. The mixture was concentrated by vacuum distillation. The residue was diluted with dichloromethane (100 mL). The organic phase was washed with NaHCO₃ (sat, aq) containing Na₂S₂O₃. The aqueous phase was extracted with dichloromethane. The combined organic phases were dried (MgSO₄) and evaporated to give 4-bromo-2-fluoro-6-methylphenol (1.360 g, 88% yield):

¹H NMR (500 MHz, DMSO-d6) δ ppm 9.66 (br. s., 1H) 7.25 (dd, 1H) 7.12 (s, 1H) 2.16 (s, 3H); MS (EI+) m/z 204, 206 [M⁺].

### Example 29i

### 5-Bromo-1-fluoro-2-methoxy-3-methylbenzene

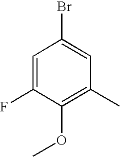

4-Bromo-2-fluoro-6-methylphenol (0.34 g, 1.66 mmol) was dissolved in acetonitrile (10 mL). Potassium carbonate (0.458 g, 3.32 mmol) was added followed by iodomethane (0.207 mL, 3.32 mmol). The mixture was stirred at RT over the week end. The mixture was diluted with dichloromethane and washed with brine. The aqueous phase was extracted with dichloromethane. The combined organic phases were dried and evaporated. The residue was purified by column chromatography on silica eluting with gradients of EtOAc in heptane. The fractions containing product were pooled and the solvents were evaporated to give 5-bromo-1-fluoro-2-methoxy-3-methylbenzene (0.150 g, 41% yield):

¹H NMR (500 MHz, CDCl₃) δ ppm 7.07-7.12 (m, 2H) 3.89 (d, 3H) 2.25 (s, 3H); MS (EI+) m/z 218, 220 [M⁺].

### Example 30i

### 5-(3-Bromophenyl)-5-(3-fluoro-4-methoxy-5-methylphenyl)-5H-pyrrolo[3,4-b]pyridin-7-amine

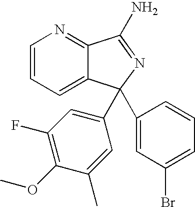

n-Butyllithium (0.278 mL, 0.70 mmol) was added dropwise to a solution of 5-bromo-1-fluoro-2-methoxy-3-methylbenzene (129 mg, 0.59 mmol) in THF (1 mL) at −78° C. under argon atmosphere. The mixture was stirred for 5 min and then a solution of N-((3-bromophenyl)(2-cyanopyridin-3-yl)methylene)-2-methylpropane-2-sulfinamide (209 mg, 0.54 mmol) in THF (1 mL) was added dropwise. The resulting mixture was stirred at −78° C. for 15 min and then the cooling bath was removed and the mixture was stirred at r.t. for 3.5 h. HCl (0.5 M in MeOH) (1.606 mL, 2.01 mmol) was added and the mixture was stirred at r.t. for 1 h. NaHCO₃ (aq sat) was added and the mixture was extracted with EtOAc (×3), dried (Na₂SO₄), filtered and concentrated. Purification by silica gel flash chromatography CHCl₃/MeOH 20:1 gave 5-(3-bromophenyl)-5-(3-fluoro-4-methoxy-5-methylphenyl)-5H-pyrrolo[3,4-b]pyridin-7-amine (79 mg, 35% yield):

¹H NMR (400 MHz, DMSO-d₆) δ ppm 8.64 (dd, 1H), 8.36 (dd, 1H), 7.39-7.52 (m, 3H), 7.35 (m, 1H), 7.25 (m, 1H), 6.96-7.03 (m, 2H), 6.88 (br. s., 2H), 3.76 (d, 3H), 2.16 (s, 3H); MS (ES+) m/z 426, 428 [M+1]⁺.

### Example 31i

### 5-(3-Bromophenyl)-5-(2-(2,2,2-trifluoroethoxy)pyridin-4-yl)-5H-pyrrolo[3,4-b]pyridin-7-amine

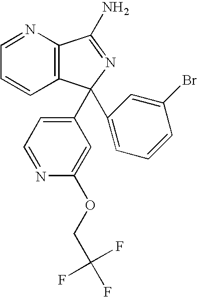

tert-Butyllithium (1.6 M in pentane) (1.922 mL, 3.07 mmol) was dropwise added to dry THF (10.00 mL) under argon at −100° C. 4-Bromo-2-(2,2,2-trifluoroethoxy)pyridine (0.328 g, 1.28 mmol) in dry THF (5.000 mL) was added dropwise. The mixture was stirred at −100° C. for 5 min, then N-((3-bromophenyl)(2-cyanopyridin-3-yl)methylene)-2-methylpropane-2-sulfinamide (0.500 g, 1.28 mmol) in dry THF (5.000 mL) was added dropwise. The reaction mixture was stirred at −100° C. for 30 min, then at −70° C. for 2 h. Methanol (5.00 mL) was added and stirring continued for 30 min at −70° C. The cooling bath was removed and stirring continued for additional 30 min. The reaction mixture was concentrated in vacuo. The residue was partitioned between aqueous sodium bicarbonate (sat.) and dichloromethane (×3). The combined organic layers were dried (Na₂SO₄), filtered and concentrated in vacuo. The residue was filtered through a syringe filter and purified by prep-HPLC to give 5-(3-bromophenyl)-5-(2-(2,2,2-trifluoroethoxy)pyridin-4-yl)-5H-pyrrolo[3,4-b]pyridin-7-amine (0.110 g, 18% yield):

¹H NMR (400 MHz, DMSO-d₆) δ ppm 8.66-8.69 (m, 1H) 8.40-8.46 (m, 1H) 8.10 (d, 1H) 7.43-7.54 (m, 3H) 7.36-7.42 (m, 1H) 7.28 (t, 1H) 7.08 (dd, 1H) 7.02 (br. s., 2H) 6.81 (dd, 1H) 4.93 (q, 2H); MS (ES+) m/z 463, 465 [M+1]⁺.

### Example 32i

### 5-(3-Bromophenyl)-5-(2-(2,2-difluoroyinyloxy)pyridin-4-yl)-5H-pyrrolo[3,4-b]pyridin-7-amine bis(2,2,2-trifluoroacetic acid)

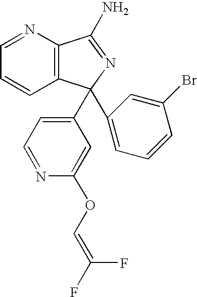

tert-Butyllithium (1.6 M in pentane) (1.922 mL, 3.07 mmol) was dropwise added to dry THF (10.00 mL) under argon at −100° C. 4-Bromo-2-(2,2,2-trifluoroethoxy)pyridine (0.328 g, 1.28 mmol) in dry THF (5.000 mL) was added dropwise. The mixture was stirred at −100° C. for 5 min, then N-((3-bromophenyl)(2-cyanopyridin-3-yl)methylene)-2-methylpropane-2-sulfinamide (0.500 g, 1.28 mmol) in dry THF (5.000 mL) was added dropwise. The reaction was stirred at −100° C. for 30 min, then at −70° C. for 1 h. Hydrochloric acid (0.5 M in methanol) (7.69 mL, 3.84 mmol) was added. The mixture was stirred over night while and was allowed to reach room temperature during this time. The reaction mixture was concentrated in vacuo. The residue was partitioned between aqueous sodium bicarbonate (sat.) and dichloromethane (×3). The combined organic layers were dried (Na₂SO₄), filtered and concentrated in vacuo. Purification by silica chromatography using 0 to 10% (3.5 M ammonia in methanol) in dichloromethane followed by prep-HPLC (Column Gemini NX C18; 21*250 mm; 5 μm; Mobilphase: 20-60% MeCN/H₂0+0.1% TFA; Flowrate: 20 ml/min) gave 5-(3-bromophenyl)-5-(2-(2,2-difluorovinyloxy)pyridin-4-yl)-5H-pyrrolo[3,4-b]pyridin-7-amine (0.064 g, 7% yield):

¹H NMR (400 MHz, DMSO-d₆) δ ppm 12.28 (br. s., 1H) 10.44 (br. s., 1H) 10.08 (br. s., 1H) 8.99 (dd, 1H) 8.57 (dd, 1H) 8.29 (dd, 1H) 7.91 (dd, 1H) 7.66 (ddd, 1H) 7.47 (t, 1H) 7.40 (t, 1H) 7.24-7.33 (m, 2H) 7.19 (dd, 1H) 7.01-7.06 (m, 1H); MS (ES+) m/z 443, 445 [M+1]⁺.

### Example 33i

### 5-(3-Bromophenyl)-5-(4-(difluoromethoxy)-3-fluorophenyl)-5H-pyrrolo[3,4-b]pyridin-7-amine

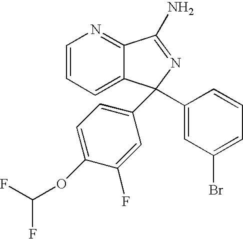

The title compound was synthesized as described for Example 5i in 21% yield starting from N-((3-bromophenyl)(2-cyanopyridin-3-yl)methylene)-2-methylpropane-2-sulfinamide (0.15 g, 0.38 mmol) and 4-bromo-1-(difluoromethoxy)-2-fluorobenzene (0.111 g, 0.46 mmol). It was used in the next reaction without purification.

### Example 34i

### 5-Bromo-2-fluoromethoxy-1,3-dimethyl-benzene

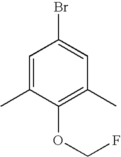

NaH (60% dispersion in oil, 1.75 g, 43.8 mmol) was added in small portions to a solution of 4-bromo-2,6-dimethyl-phenol (8.09 g, 39.79 mmol) in dry DMF (80 mL) and the resulting mixture was stirred at room temperature for 15 minutes. Chloro-fluoro-methane gas was bubbled through the above solution for 10 minutes (approximately 15 grams, 219 mmol was added), the pressure tube was then sealed and the reaction mixture was heated at 80° C. for 3 hours. The reaction mixture was cooled to room temperature, diluted with water (200 mL) and extracted with diethyl ether (2×200 mL). The combined extracts were washed with water (3×100 mL), brine, dried over magnesium sulfate and concentrated in vacuo to afford 9.8 g (quantitative yield) of the title compound:

¹H NMR (400 MHz, CDCl₃) δ ppm 7.19 (s, 2H), 5.59 (d, J_{HF}=54.9 Hz), 2.25 (s, 6H); ¹⁹F NMR (376 MHz, CDCl₃) δ ppm-148.13; CHN: Calcd for C9H10BrFO+0.2C6H14: C, 48.94; H, 5.15. Found: C, 48.82; H, 5.28.

### Example 35i

### 5-(3-Bromophenyl)-5-(4-(fluoromethoxy)-3,5-dimethylphenyl)-5H-pyrrolo[3,4-b]pyridin-7-amine

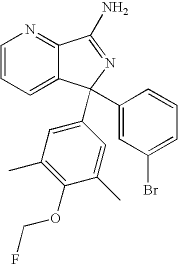

5-Bromo-2-(fluoromethoxy)-1,3-dimethylbenzene (0.328 mL, 2.25 mmol) was dissolved in THF (20 mL) under nitrogen atmosphere and cooled to ±78° C. n-Butyllithium (1.640 mL, 4.10 mmol) was added and the reaction was stirred for 1.5 h. N-((3-bromophenyl)(2-cyanopyridin-3-yl)methylene)-2-methylpropane-2-sulfinamide (0.8 g, 2.05 mmol) in THF (5 mL) was added and the reaction was stirred at −78° C. for 30 minutes and then allowed to reach room temperature. Water, NaHCO₃ (aq) and EtOAc were added and the organics were collected and concentrated. The residue was redissolved in methanol (10 mL), and hydrogen chloride (1M in diethyl ether) (4.10 mL, 4.10 mmol) was added. The reaction was stirred at ambient temperature over night. The solution was made basic with ammonia. The water was added and the product was extracted with DCM. The organics were dried over Mg₂SO₄, concentrated and purified with preparative HPLC to give the title compound (200 mg, 22% yield):

¹H NMR (500 MHz, CDCl₃) δ ppm 8.65 (d, 1H) 7.90 (d, 1H) 7.46 (s, 1H) 7.35-7.43 (m, 2H) 7.29 (br. s., 1H) 7.12-7.20 (m, 1H) 6.93 (s, 2H) 5.58 (s, 1H) 5.47 (s, 1H) 2.21 (s, 6H); MS (ES+) m/z 440, 442 [M+1]⁺.

### Example 36i

### 4-Bromo-2-(3-fluoropropoxy)pyridine

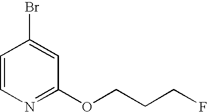

4-Bromo-2-fluoropyridine (2 g, 11.36 mmol) and 3-fluoropropan-1-ol (0.854 mL, 11.36 mmol) were dissolved in dry THF (20 mL) under argon. The solution was cooled with an external ice/water bath and held at 0° C. Potassium tert-butoxide (1.275 g, 11.36 mmol) was added in portions during 20 min with efficient stirring. The resulting solution was stirred further at 0° C. for 30 mins, whereafter the cooling bath was removed and the mixture stirred at ambient temperature over night. The reaction was quenched by addition of water (15 mL) and the phases were separated. The aqueous layer was extracted twice with diethyl ether and the combined organic extracts were washed with brine, dried (MgSO₄), filtered and concentrated. The mixture was purified by silica gel column chromatography (0-100% ethyl acetate in heptane) to give 1.83 g (69% yield) of the title compound:

¹H NMR (600 MHz, CDCl₃) δ ppm 7.98 (d, 1H) 7.03 (dd, 1H) 6.95 (d, 1H) 4.55-4.69 (m, 2H) 4.43 (t, 2H) 2.10-2.23 (m, 2H); MS (EI) m/z 233, 235 [M⁺].

### Example 37i

### 5-(3-Bromophenyl)-5-(2-(3-fluoropropoxy)pyridin-4-yl)-5H-pyrrolo[3,4-b]pyridin-7-amine

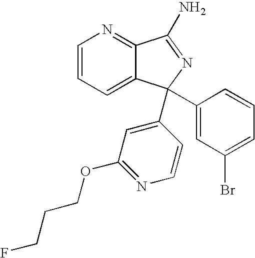

tert-Butyllithium (3.46 mL, 5.53 mmol) was added dropwise to THF (20 mL) at −100° C. under an argon atmosphere (yellow solution). A solution of 4-bromo-2-(3-fluoropropoxy)pyridine (540 mg, 2.31 mmol) in THF (5 mL) was added dropwise followed by the addition of N-((3-bromophenyl)(2-cyanopyridin-3-yl)methylene)-2-methylpropane-2-sulfinamide (900 mg, 2.31 mmol) in THF (5 mL). The resulting reaction mixture was left on the thawing cooling bath for 30 min then the cooling bath was removed and the mixture was stirred at r.t. for 1 h. Hydrogen chloride (11.53 mL, 11.53 mmol) was added and the resulting mixture was stirred at r.t. for 1 h. The mixture was separated between water and EtOAc, the organics were collected and concentrated to give the crude title compound (1 g, 98% yield), which was used as such in the next step:

MS (ES+) m/z 441, 443 [M+1]⁺.

### Example 38i

### 2-Chloro-isonicotinoyl chloride

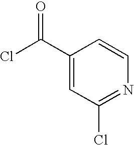

A mixture of 2-chloro-isonicotinic acid (25 g, 158.7 mmol), SOCl₂ (150 mL) and 5 drops of DMF was heated to reflux for 24 hours. The volatiles were removed under reduced pressure and the crude product was purified by distillation to afford 20 g (72% yield) of 2-chloro-isonicotinoyl chloride:

¹H NMR (400 MHz, CDCl₃) δ ppm 8.66 (d, 1H) 7.95 (s, 1H) 7.83 (dd, 1H).

### Example 39i

### 3-(2-Chloro-pyridine-4-carbonyl)-pyridine-2-carbonitrile

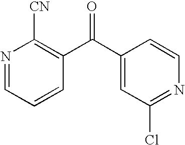

A solution of 3-bromo-pyridine-2-carbonitrile (5.0 g, 27.3 mmol) in dry THF (50 mL) was added dropwise over 15 min to Rieke Zinc (5.0 g, 76.49 mmol) in THF (50 mL) under nitrogen atmosphere. The mixture was stirred at room temperature for 5 hours and allowed to stand at −20° C. for 24 hours. The solution of 2-cyanopyridinezinc bromide was carefully decanted to remove the excess of zinc. 2-Chloro-isonicotinoyl chloride (5.3 g, 30.1 mmol), followed by Pd(PPh₃)₂Cl₂ (0.96 g, 1.37 mmol) were added to the solution of 2-cyanopyridinezinc bromide (100 mL, ˜27.32 mmol) and the resulting mixture was stirred at room temperature for 4 hours. Ethyl acetate (80 mL) and H₂O (40 mL) were then added and the phases separated. The organic layer was washed with H₂O (3×30 mL), dried over Na₂SO₄, filtered and concentrated under reduced pressure. The residue was purified by flash column chromatography using a gradient of 20-50% EtOAc in hexane to afford 2 g (30% yield) of the title compound:

¹H NMR (400 MHz, CDCl₃) δ ppm 8.96 (dd, 1H) 8.66 (d, 1H) 8.00 (dd, 1H) 7.72 (dd, 1 H) 7.63 (s, 1H) 7.52-7.55 (m, 1H); MS (ES+) m/z: 244.0 [M+1]⁺.

### Example 40i

### 2-Methyl-propane-2-sulfinic acid (2-chloro-pyridin-4-yl)-(2-cyano-pyridin-3-yl)-methyleneamide

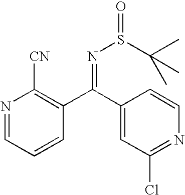

Ti(OEt)₄ (15 mL, 71.5 mmol) was added to a solution of 3-(2-chloro-pyridine-4-carbonyl)-pyridine-2-carbonitrile (4.0 g, 16.42 mmol) and 2-methyl-propane-2-sulfinic acid amide (3.58 g, 29.55 mmol) in dry THF (100 mL) at room temperature. The resulting mixture was heated to reflux for 40 hours. Methanol (50 mL) and a saturated solution of Na₂CO₃ (10 mL) were added and the resulting suspension was filtered through a pad of Celite. The solids were washed with THF (50 mL) and CH₃OH (20 mL) and the filtrate was concentrated under reduced pressure. The residue was purified by flash column chromatography using a gradient of 25-50% EtOAc in hexane to afford 2.2 g (38% yield) of the title compound:

¹H NMR (400 MHz, CDCl₃) δ ppm 8.81-8.86 (m, 1H) 8.53 (d, 1H) 7.74 (d, 1H) 7.64 (dd, 1H) 7.38 (s, 1H) 7.32 (d, 1H) 1.41 (s, 9H); MS (ES+) m/z: 347.16 [M+1]⁺.

### Example 41i

### 5-(2-Chloro-pyridin-4-yl)-5-(4-difluoromethoxy-3,5-dimethyl-phenyl)-5H-pyrrolo[3,4-b]pyridin-7-ylamine

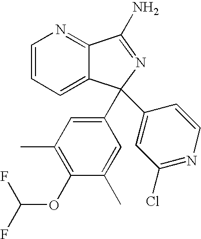

n-Butyllithium (2.5M in hexane, 0.26 mL, 0.64 mmol) was added dropwise to a solution of 5-bromo-2-difluoromethoxy-1,3-dimethyl-benzene (146 mg, 0.58 mmol) in dry THF (2 mL) at −78° C. The reaction mixture was stirred for 5 minutes and a solution of 2-methyl-propane-2-sulfinic acid (2-chloro-pyridin-4-yl)-(2-cyano-pyridin-3-yl)-methyleneamide 2 (151 mg, 0.44 mmol, prepared as described in Example 15, step iii) in dry THF (1 mL) was added dropwise at −78° C. The stirring was continued for 1 hour and methanolic HCl (1.25M, 2 mL, 2.5 mmol) was added at −78° C. The mixture was allowed to warm slowly to room temperature and stirred overnight. The mixture was treated with saturated NaHCO₃ solution (20 mL) and extracted with ethyl acetate (2×20 mL). The combined extracts were washed with H₂O, brine, dried over Na₂SO₄ and concentrated under reduced pressure. The residue was purified by flash column chromatography using 5% CH₃OH in DCM to afford 85 mg (46% yield) of the title compound:

¹H NMR (400 MHz, CDCl₃) δ ppm 8.67 (dd, 1H) 8.29 (d, 1H) 7.89 (dd, 1H) 7.40 (dd, 1 H) 7.28 (d, 1H) 7.19 (dd, 1H) 6.92 (s, 2H) 6.30 (t, 1H) 5.56 (br. s., 2H) 2.24 (s, 6H); MS (ES+) m/z: 415.16, 417.14 [M+1]⁺.

### Example 42i

### 2-Allyloxy-1-bromo-3-methyl-benzene

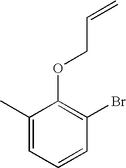

NaH (60% suspension in mineral oil, 5.1 g, 128.31 mmol) was added in small portions to a solution of 2-bromo-6-methylphenol (20 g, 106.9 mmol) in anhydrous DMF (200 mL) at 0° C. The reaction mixture was stirred for 5 minutes and allyl bromide (10.9 mL, 128.3 mmol) was added dropwise. The resulting mixture was warmed to room temperature and stirred overnight. Water (200 mL) was added and the mixture was extracted with diethyl ether (2×300 mL). The combined extracts were washed with water, brine, dried over sodium sulfate and concentrated under reduced pressure. The crude product was purified by flash column chromatography using 5% ethyl acetate in hexanes to afford 24.5 g of the title compound:

¹H NMR (400 MHz, CDCl₃) δ ppm 7.40 (dd, 1H), 7.13 (d, 1H), 6.90 (t, 1H), 6.20-6.10 (m, 1H), 5.47 (dq, 1H), 5.30 (dq, 1H), 4.45 (dt, 2H), 2.32 (s, 3H).

### Example 43i

### 2-Cyclopropyl-6-methyl-phenol

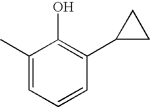

t-BuLi (1.7 M in pentane, 64 mL, 108.3 mmol) was added dropwise to a solution of 2-allyloxy-1-bromo-3-methyl-benzene (12.0 g, 52.8 mmol) in anhydrous diethyl ether (300 mL) at −78° C. The mixture was stirred at −78° C. for 30 minutes and TMEDA (17.5 mL, 116.3 mmol) was added slowly. The resulting mixture was stirred at −78° C. for 45 minutes, then warmed to room temperature and stirred overnight. Water (300 mL) was added and the mixture extracted with ethyl acetate (2×300 mL). The combined extracts were washed with 2 N HCl (150 mL), brine, dried over sodium sulfate and concentrated under reduced pressure. The residue was purified by flash column chromatography using 10% ethyl acetate in hexanes to afford 7.0 g (89% yield) of the title compound:

¹H NMR (400 MHz, CDCl₃) δ ppm 7.01 (d, 1H), 6.96 (d, 1H), 6.76 (t, 1H), 5.55 (s, OH), 2.26 (s, 3H), 1.79-1.73 (m, 1H), 0.99-0.94 (m, 2H), 0.65-0.62 (m, 2H).

### Example 44i

### 4-Bromo-2-cyclopropyl-6-methyl-phenol

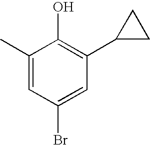

Bromine (1.6 mL, 31.71 mmol) was added dropwise to a solution of 2-cyclopropyl-6-methyl-phenol (4.7 g, 31.71 mmol) in dichloromethane (50 mL) at 0° C. The reaction mixture was allowed to warm to room temperature over 1 hour. Dichloromethane (50 mL) was then added and the mixture was washed with saturated NaHCO₃ solution, brine, dried over sodium sulfate and concentrated under reduced pressure. The crude product was purified by flash column chromatography using 10% ethyl acetate in hexanes to afford 5.5 g (76% yield) of the title compound:

¹H NMR (400 MHz, CDCl₃) δ ppm 7.13 (d, 1H), 7.05 (d, 1H), 5.50 (s, OH), 2.22 (s, 3H), 1.76-1.72 (m, 1H), 1.00-0.96 (m, 2H), 0.65-0.62 (m, 2H).

### Example 45i

### 5-Bromo-1-cyclopropyl-2-difluoromethoxy-3-methyl-benzene

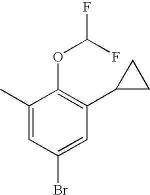

A solution of 4-bromo-2-cyclopropyl-6-methyl-phenol (5.5 g, 24.22 mmol) in a mixture of isopropanol and 20% NaOH (200 mL, 1:1) was heated to 40° C. Chlorodifluoromethane gas was bubbled continuously into the solution at a moderate rate for 5 hours. The mixture was then cooled to room temperature and extracted with diethyl ether (2×300 mL). The combined extracts were washed with water (2×300 mL), brine, dried over sodium sulfate, and concentrated under reduced pressure. The crude product was purified by flash column chromatography using 5% ethyl acetate in hexanes to afford 5.3 g (79% yield) of the title compound:

¹H NMR (400 MHz, CDCl₃) δ ppm 7.17 (d, 1H), 6.83 (d, 1H), 6.43 (t, 1H), 2.29 (s, 3 H), 2.12-2.07 (m, 1H), 1.05-1.00 (m, 2H), 0.71-0.67 (m, 2H). Elemental analysis: Calcd for C₁₁K₁₁BrF₂O: C, 47.68; H, 4.00; N, 0.00. Found: C, 48.42; H, 4.07; N, 1.3.

### Example 46i

### 5-(2-Chloro-pyridin-4-yl)-5-(3-cyclopropyl-4-difluoromethoxy-5-methyl-phenyl)-5H-pyrrolo[3,4-b]pyridin-7-ylamine

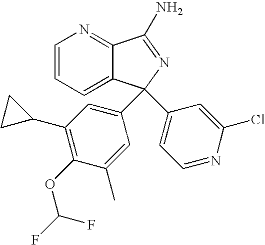

n-Butyllithium (2.5M in hexane, 0.14 mL, 0.36 mmol) was added dropwise to a solution of 5-bromo-1-cyclopropyl-2-difluoromethoxy-3-methyl-benzene (0.1 g, 0.361 mmol) in dry THF (1 mL) at −78° C. The reaction mixture was stirred for 5 minutes and a solution of 2-methyl-propane-2-sulfuric acid (2-chloro-pyridin-4-yl)-(2-cyano-pyridin-3-yl)-methyleneamide (0.083 g, 0.24 mmol) in dry THF (1 mL) was added dropwise at −78° C. The stirring was continued for 1 hour and methanolic HCl (1.25M, 1.1 mL) was added at −78° C. The mixture was allowed to warm slowly to room temperature and stirred overnight. The mixture was treated with dichloromethane (20 mL) and saturated NaHCO₃ solution (50 mL). The organic layer was washed with H₂O, dried over Na₂SO₄, filtered and concentrated under reduced pressure. The residue was purified by flash column chromatography using 5% CH₃OH in DCM to afford 50 mg (50% yield) of the title compound:

¹H NMR (400 MHz, CDCl₃) δ ppm 8.65-8.68 (m, 1H) 8.28 (d, 1H) 7.83-7.87 (m, 1H) 7.39 (dd, 1H) 7.24 (s, 1H) 7.14 (dd, 1H) 6.90 (d, 1H) 6.60-6.64 (m, 1H) 6.43 (s, 1H) 5.43 (br. s., 2H) 2.25 (s, 3H) 2.05-2.11 (m, 1H) 0.95 (dd, 2H) 0.48-0.59 (m, 2H); MS (ES+) m/z: 441.17 [M+1]⁺.

### Example 47i

### 5-(3-Bromo-phenyl)-5-(3-cyclopropyl-4-difluoromethoxy-5-methyl-phenyl)-5H-pyrrolo[3,4-b]pyridin-7-ylamine

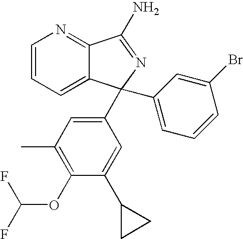

n-Butyllithium (2.5 M in hexanes, 0.41 mL, 1.025 mmol) was added dropwise to a solution of 5-bromo-1-cyclopropyl-2-difluoromethoxy-3-methyl-benzene (284 mg, 1.03 mmol) in THF (2 mL) at −78° C. under nitrogen atmosphere. The reaction mixture was stirred for 5 minutes, 2-methyl-propane-2-sulfinic acid-(3-bromo-phenyl)-(2-cyano-pyridin-3-yl)-methyleneamide (200 mg, 0.51 mmol) in THF (3 mL) was added dropwise at −78° C., and the stirring was continued for 1 hour. Methanolic HCl (1.25M, 2.5 mL) was added at −30° C. and the reaction mixture was allowed to slowly warm to room temperature. The reaction mixture was then partitioned between water and ethyl acetate. The aqueous phase was separated and further extracted with ethyl acetate (3×20 mL). The combined extracts were washed with brine, dried over anhydrous MgSO₄, filtered and concentrated in vacuo. The residue was purified by flash chromatography using a gradient of 0-5% MeOH in dichloromethane to afford the tile compound (0.145 g, 58% yield):

¹H NMR (400 MHz, CDCl₃) δ ppm 8.84 (d, 1H) 7.96 (d, 1H) 7.74-7.64 (m, 1H) 7.49 (d, 1H) 7.32 (s, 1H) 7.25-7.27 (m, 2H) 6.88 (d, 1H) 6.65 (d, 1H) 6.25-6.49 (m, 1H) 5.30 (s, 2H) 2.27 (s, 3H) 2.03-2.14 (m, 1H) 0.95-1.04 (m, 2H) 0.62 (q, 2H); MS (ES+)

m/z: 485 [M+1]⁺.

### Example 48i

### 1-Allyloxy-2-bromo-benzene

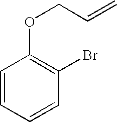

NaH (60% suspension in mineral oil, 2.4 g, 60.0 mmol) was added in small portions to a solution of 2-bromo-phenol (10.0 g, 57.8 mmol) in dry DMF (100 mL) at 0° C. The reaction mixture was stirred vigorously for 1 hour at 0° C. and allyl bromide (5.8 mL, 68.0 mmol) was added slowly to the reaction mixture. The reaction mixture was allowed to warm to room temperature and stirred for 1 hour. Ice-cold saturated NH₄Cl solution (100 mL) was then added and the mixture was extracted with Et₂O (3×150 mL). The combined extracts were washed with water, dried over MgSO₄, filtered and concentrated under reduced pressure. The oily residue was purified by flash column chromatography using 5% EtOAc in hexane followed by careful concentration at lower temperature of the fractions (to avoid possible loss of the material) to afford the title compound (10.5 g, 85% yield):

¹H NMR (400 MHz, CDCl₃) δ ppm 7.48-7.57 (m, 1H), 7.23-7.28 (m, 1H), 6.76-6.95 (m, 2H), 5.98-6.14 (m, 1H), 5.41-5.54 (m, 1H), 5.31 (dd, 1H), 4.62 (d, 2H).

### Example 49i

### 2-Cyclopropyl-phenol

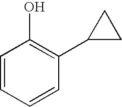

t-BuLi (1.7 M in pentane, 77.0 mL, 130.9 mmol) was added dropwise to a solution of 1-allyloxy-2-bromo-benzene (14.0 g, 65.7 mmol) in anhydrous Et₂O (300 mL) over a period of 1 hour at −78° C. The reaction mixture was stirred for 1 hour at −78° C. and TMEDA (22.6 mL, 150.7 mmol) was then added slowly. The reaction mixture was allowed to warm to room temperature and stirred overnight. Ice-cold saturated NH₄Cl solution (100 mL) was added and the resulting mixture was extracted with EtOAc (3×200 mL). The combined extracts were dried over MgSO₄, filtered and concentrated under reduced pressure. The residue was purified by flash column chromatography using 10% EtOAc in hexane. The fractions were concentrated carefully at lower temperature (to avoid possible loss of the material) to afford 2-cyclopropyl-phenol (8.0 g, 90% yield).

¹H NMR (400 MHz, CDCl₃) δ ppm 7.10-7.16 (m, 1H), 7.08 (d, 1H), 6.82-6.88 (m, 2 H), 5.46 (br. s., 1H), 1.74-1.86 (m, 1H), 0.91-1.02 (m, 2H), 0.59-0.69 (m, 2H).

### Example 50i

### 4-Bromo-2-cyclopropyl-phenol

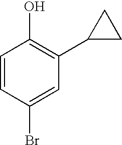

Bromine (3.06 mL, 59.7 mmol) was added dropwise to a solution of 2-cyclopropyl-phenol (8.0 g, 59.7 mmol) in CH₂Cl₂ (300 mL) at 0° C. The reaction mixture was stirred for 1 hour at 0° C. and then quenched using saturated NaHCO₃ solution. The organic phase was separated and the aqueous layer was further extracted with CH₂Cl₂ (3×50 mL). The combined extracts were dried over MgSO₄, filtered and concentrated under reduced pressure. The residue was purified by flash column chromatography using 10% EtOAc in hexane to afford 4-bromo-2-cyclopropyl-phenol (12.1 g, 95% yield):

¹H NMR (400 MHz, CDCl₃) δ ppm 7.21 (dd, 1H), 7.16 (d, 1H), 6.74 (d, 1H), 5.58 (s, 1 H), 1.76-1.85 (m, 1H), 0.95-1.02 (m, 2H), 0.61-0.69 (m, 2H).

### Example 51i

### 4-Bromo-2-cyclopropyl-1-methoxy-benzene

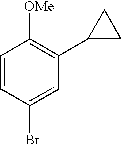

K₂CO₃ (8.7 g, 62.9 mmol) was added to a solution of 4-bromo-2-cyclopropyl-phenol (9.0 g, 42.3 mmol) in DMF (40 mL) at 0° C., followed by addition of MeI (3.7 mL 59.4 mmol). The reaction mixture was allowed to warm to room temperature and stirred for 2 hours.

The reaction mixture was filtered, diluted with H₂O (100 mL) and extracted with Et₂O (3×50 mL). The combined extracts were dried over MgSO₄, filtered and concentrated under reduced pressure. The residue was purified by flash column chromatography using 3% EtOAc in hexane to give 4-bromo-2-cyclopropyl-1-methoxy-benzene (8.0 g, 84% yield): ¹H NMR (400 MHz, CDCl₃) δ ppm 7.21 (dd, 1H), 6.91 (d, 1H), 6.70 (d, 1H), 3.84 (s, 3 H), 2.13 (tt, 1H), 0.83-1.03 (m, 2H), 0.53-0.70 (m, 2H); MS (ES+) m/z: 227 [M+1]⁺.

### Example 52i

### 7-(3-Bromo-phenyl)-7-(3-cyclopropyl-4-methoxy-phenyl)-7H-pyrrolo[3,4-b]pyridin-5-ylamine

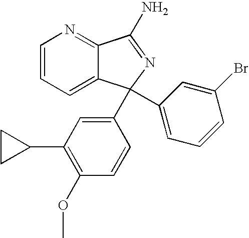

n-Butyllithium (2.5 M in hexanes, 0.5 mL, 1.24 mmol) was added dropwise to a solution 4-bromo-2-cyclopropyl-1-methoxy-benzene (256 mg, 1.13 mmol) in THF (2 mL) at −78° C. under nitrogen atmosphere. The reaction mixture was stirred for 5 minutes and N-((3-Bromophenyl)(2-cyanopyridin-3-yl)methylene)-2-methylpropane-2-sulfinamide (200 mg, 0.51 mmol) dissolved in THF (3 mL) was added dropwise. The reaction mixture and stirred at −78° C. for 1 hour, then quenched with methanolic HCl (1.25 M, 2.5 mL) at −30° C. and allowed to warm slowly to room temperature. The mixture was partitioned between water and ethyl acetate (3×20 mL) and the phases were separated. The organic phase was washed with brine, dried over anhydrous MgSO₄, filtered and concentrated in vacuo. The is residue was purified by flash chromatography using a gradient of 0-5% MeOH in dichloromethane to afford 140 mg (63% yield) of the title compound:

¹H NMR (400 MHz, CDCl₃) δ ppm 8.60 (d, 1H) 7.83 (d, 1H) 7.42 (s, 1H) 7.30-7.40 (m, 2H) 7.21 (d, 1H) 7.12 (d, 1H) 7.03 (dd, 1H) 6.78 (d, 1H) 6.72 (d, 1H) 5.30 (s, 2H) 3.82 (s, 3H) 2.08 (t, 1H) 0.84 (d, 2H) 0.45-0.58 (m, 2H); MS (ES+) m/z: 436, 434 [M+1]⁺.

### Example 53i

### 4-Bromo-2-(2-hydroxy-ethyl)-phenol

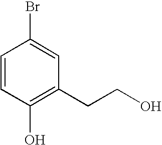

Concentrated H₂SO₄ (0.7 mL, 12.7 mmol) and NBS (49.6 g, 278.6 mmol) were added to a solution of 2-hydroxyphenethyl alcohol (35.0 g, 253.3 mmol) in dry THF (500 mL) at −25° C. The mixture was allowed to warm to room temperature and stirred overnight. Aqueous sodium thiosulfite (10%, 70 mL) and water (200 ml) were added and the resulting mixture was extracted with ethyl acetate (2×400 mL). The combined organic extracts were washed with brine, dried over sodium sulfate and concentrated under reduced pressure. The residue was purified by flash column chromatography using 50% ethyl acetate in hexane to afford 55.0 g of the title compound:

¹H NMR (400 MHz, CDCl₃) δ ppm 8.03 (br, s, OH), 7.25-7.22 (m, 1H), 7.18 (m, 1H), 6.80 (d, 1H), 3.99 (t, 2H), 2.85 (t, 2H), 2.50 (br, s, OH).

### Example 54i

### 2-(5-Bromo-2-difluoromethoxy-phenyl)-ethanol

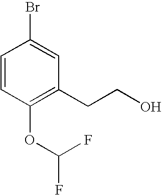

A mixture 4-bromo-2-(2-hydroxy-ethyl)-phenol (45.8 g, 211.0 mmol), potassium carbonate (116.6 g, 844.0 mmol) and sodium chlorodifluoroacetate (35.4 g, 232.1 mmol) in a mixture of DMF-water (440 mL, 10:1) was heated at 120° C. overnight. The reaction mixture was cooled to room temperature, water (500 mL) was added and the mixture was extracted with ethyl acetate (2×500 mL). The combined extracts were washed with water (2×500 mL), brine, dried over sodium sulfate and concentrated under reduced pressure. The crude product was purified by flash column chromatography using 30% ethyl acetate in hexane to afford 18.5 g (33% yield) of the title compound:

¹H NMR (400 MHz, CDCl₃) δ ppm 7.45 (d, 1H), 7.37 (dd, 1H), 7.01 (d, 1H), 6.50 (t, 1 H), 3.87 (t, 2H), 2.92 (t, 2H); ¹⁹F NMR (376 MHz, CDCl₃) δ ppm-80.7 (d, J=74.6 Hz).

### Example 55i

### 4-Bromo-1-difluoromethoxy-2-(2-fluoro-ethyl)-benzene

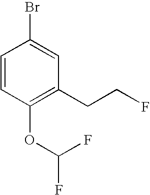

DAST (10.2 mL, 83.1 mmol) was added to a solution of 2-(5-bromo-2-difluoromethoxy-phenyl)-ethanol (18.5 g, 69.3 mmol) in dry dichloromethane (150 mL) at −40° C. The mixture was allowed to warm to room temperature and the volatiles were removed under reduced pressure. The residue was purified by flash column chromatography using a gradient of 3-10% ethyl acetate in hexane to afford 4.6 g (24% yield) of the title compound:

¹H NMR (400 MHz, CDCl₃) δ ppm 7.45 (s, 1H), 7.39 (d, 1H), 7.02 (d, 1H), 6.50 (t, 1H), 4.69 (dt, 2H), 3.07 (dt, 2H); ¹⁹F NMR (376 MHz, CDCl₃) δ ppm-80.8 (d, J=73.4 Hz), -217.4 (sep, J=24.1 Hz).

### Example 56i

### 5-(3-Bromo-phenyl)-5-[4-difluoromethoxy-3-(2-fluoro-ethyl)-phenyl]-5H-pyrrolo[3,4-b]pyridin-7-ylamine

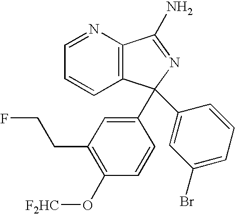

4-Bromo-1-difluoromethoxy-2-(2-fluoro-ethyl)-benzene (350 mg, 1.30 mmol) dissolved in dry THF (1 mL) was added dropwise to a solution of n-butyllithium (2.5 M in hexanes, 0.57 mL, 1.43 mmol) in dry THF (2 mL) at −78° C. under nitrogen atmosphere. The reaction mixture was stirred for 2 minutes and a solution of N-((3-Bromophenyl)(2-cyanopyridin-3-yl)methylene)-2-methylpropane-2-sulfinamide (253.7 mg, 0.65 mmol) in THF (2 mL) was added slowly. The reaction mixture was stirred first at −78° C. for 1 hour and then at room temperature for 1.5 hours. Methanolic HCl (1.25M, 3 mL, 3.75 mmol) was added and the resulting mixture was stirred at room temperature for 5 hours. The volatiles were removed in vacuo and the residue was partitioned between water and ethyl acetate. The organic phase was separated, washed with brine, dried over Na₂SO₄ and concentrated. The residue was purified by flash chromatography using a gradient of 1-3% MeOH in dichloromethane to afford 494 mg (81% yield) of the title compound:

¹H NMR (400 MHz, CDCl₃) δ ppm 8.63 (dd, 1H) 7.86 (dd, 1H) 7.43 (t, 1H) 7.31-7.40 (m, 2H) 7.09-7.25 (m, 4H) 7.00 (d, 1H) 6.48 (t, 1H) 4.54-4.65 (m, 1H) 4.42-4.53 (m, 1H) 2.90-3.08 (m, 2H); ¹⁹F NMR (376 MHz, CDCl₃) δ ppm-84.11, -219.67.

### Example 57i

### 6-Bromo-2,4-dimethyl-3-hydroxypyridine

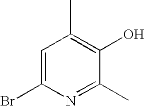

A solution of bromine (4.2 mL, 81.2 mmol) in anhydrous pyridine (80 mL) was added dropwise to a solution of 2,4-dimethyl-3-hydroxypyridine (10.0 g, 81.2 mmol) in anhydrous pyridine (160 mL). The mixture was stirred at room temperature for 1 hour, concentrated under reduced pressure and then further dried under vacuum. The residue was taken up in water (100 mL) and the resulting mixture was stirred for 0.5 hour at room temperature. The precipitated solid was collected by filtration, washed with water and air dried overnight to afford 8.7 g (53% yield) of the title compound:

¹H NMR (400 MHz, CDCl₃) δ ppm 7.10 (s, 1H), 4.73 (br, s, 1H, 2.45 (s, 3H), 2.23 (s, 3H).

### Example 58i

### 6-Bromo-3-methoxy-2,4-dimethyl-pyridine

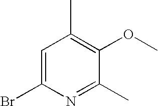

A mixture of 6-bromo-2,4-dimethyl-3-hydroxypyridine (8.7 g, 43.1 mmol), iodomethane (4.0 mL, 64.6 mmol) and potassium carbonate (11.9 g, 86.1 mmol) in acetone (250 mL) was heated at reflux temperature for 3 hours. The reaction mixture was then cooled to room temperature and filtered through a pad of Celite. The filtrate was concentrated under reduced pressure and the residue was purified by flash column chromatography using 20% ethyl acetate in hexane to afford 7.9 g (85% yield) of the title compound:

¹H NMR (400 MHz, CDCl₃) δ ppm 7.14 (s, 1H), 3.72 (s, 3H), 2.48 (s, 3H), 2.26 (s, 3H); MS (ES+) m/z: 215.96, 217.96 [M+1]⁺.

### Example 59i

### 5-(3-Bromo-phenyl)-5-(5-methoxy-4,6-dimethyl-pyridin-2-yl)-5H-pyrrolo[3,4-b]pyridin-7-ylamine

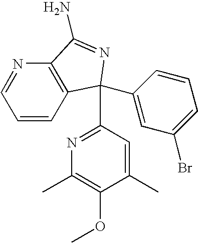

n-BuLi (2.5 M in hexanes, 0.5 mL, 1.25 mmol) was added dropwise to a solution of 6-bromo-3-methoxy-2,4-dimethyl-pyridine (0.22 g, 1.0 mmol) in anhydrous THF (1 mL) at −78° C. The mixture was stirred at −78° C. for 15 minutes and a solution of N-((3-Bromophenyl)(2-cyanopyridin-3-yl)methylene)-2-methylpropane-2-sulfinamide (0.20 g, 0.51 mmol) in THF (1 mL) was added dropwise. The reaction mixture was stirred at −78° C. for 1 hour, then warmed to −20° C. and HCl (1.25 M in MeOH, 2.4 mL, 3.0 mmol) was added. The resulting mixture was stirred at room temperature overnight, diluted with dichloromethane (20 mL) and washed with saturated NaHCO₃. The organic phase was separated, dried over sodium sulfate and concentrated under reduced pressure. The crude product was purified by flash column chromatography using 5% methanol in dichloromethane to afford 0.15 g (69% yield) of the title compound:

¹H NMR (400 MHz, CDCl₃) δ ppm 8.58 (d, 1H), 8.44 (d, 1H), 7.51 (s, 1H), 7.46 (s, 1H), 7.37-7.30 (m, 3H), 7.09 (t, 1H), 3.70 (s, 3H), 2.46 (s, 3H), 2.24 (s, 3H); MS (ES+) m/z: 422.92, 424.96 [M+1]⁺.

### Example 60i

### (2-Cyanopyridin-3-yl)zinc(II) bromide

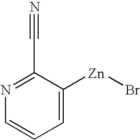

Rieke®Zinc (0.1 g/mL in THF) (100 mL, 152.93 mmol) was added, dropwise and under Ar, to a solution of 3-bromo-pyridine-2-carbonitrile (11.66 g, 63.72 mmol) in anhydrous THF (40 mL). The reaction mixture was stirred at room temperature for 3 h and stored in a freezer over the weekend while the excess zinc settled. (2-Cyanopyridin-3-yl)zinc(II) bromide (assumed quantitative yield) was used as such in the next step.

### Example 61i

### 3-Bromo-4-methoxybenzoyl chloride

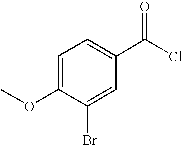

3-Bromo-4-methoxybenzoic acid (14.72 g, 63.72 mmol) was dissolved in DCM (200 mL) at room temperature, then oxalyl chloride (6.11 mL, 70.09 mmol) was added followed by DMF (five drops). The reaction mixture was stirred for 4 h. Additional oxalyl chloride (6.11 mL, 70.09 mmol) was added and the resulting mixture was stirred for 1 week. The reaction mixture was concentrated. Toluene was added and evaporated. This was repeated twice to give 3-bromo-4-methoxybenzoyl chloride (15.90 g, 100% yield), that was used without further purification.

### Example 62i

### 3-(3-Bromo-4-methoxybenzoyl)picolinonitrile

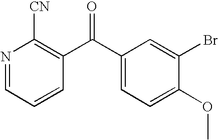

Copper (I) cyanide (5.71 g, 63.72 mmol) and lithium bromide (11.07 g, 127.44 mmol) were dissolved in THF (40 mL) and stirred for 30 min at r.t. Then the mixture was cooled to −78° C. and (2-cyanopyridin-3-yl)zinc(II) bromide (0.33 M in THF) (193 mL, 63.72 mmol) was added. The mixture was stirred at room temperature for 40 min and then cooled to −78° C. A solution of 3-bromo-4-methoxybenzoyl chloride (15.90 g, 63.72 mmol) in THF (50 mL) was added. The reaction mixture was stirred at room temperature over night. The mixture was quenched with sat.aq. NH₄Cl (15 mL) and concentrated. DCM (200 mL) and water (50 mL) was added. A precipitate was filtered off, the organic layer separated and the aqueous layer was extracted with DCM (×2). The combined organics were dried (Na₂SO₄), filtered and concentrated. Purification on a silica gel column eluted with 0-60% EtOAc in heptane gave 3-(3-bromo-4-methoxybenzoyl)picolinonitrile (6.03 g, 30% yield): MS (CI) m/z 317, 319 [M+1]⁺.

### Example 63i

### N-((3-Bromo-4-methoxyphenyl)(2-cyanopyridin-3-yl)methylene)-2-methylpropane-2-sulfinamide

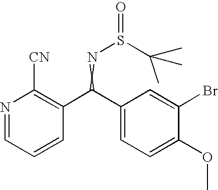

Titanium(IV) ethoxide (9.97 mL, 47.53 mmol) was added, under argon atmosphere, and at r.t., to a solution of 3-(3-bromo-4-methoxybenzoyl)picolinonitrile (6.03 g, 19.01 mmol) in dry THF (20 mL). The resulting mixture was stirred for 5 min, then 2-methylpropane-2-sulfinamide (3.00 g, 24.72 mmol) was added in one portion. The reaction was refluxed for 3 days. Methanol (10 mL), aqueous sat. sodium bicarbonate (10 mL) and ethyl acetate (20 mL) were added and the resulting mixture was stirred for 25 min. and then filtered through a pad of celite/Na₂SO₄ to remove the precipitate that formed. The filter cake was washed repeatedly with ethyl acetate. The filtrate was concentrated in vacuo and purification by silica chromatography using 0% to 50% ethyl acetate in heptane gave N-((3-bromo-4-methoxyphenyl)(2-cyanopyridin-3-yl)methylene)-2-methylpropane-2-sulfinamide (6.06 g, 76% yield):

¹H NMR (600 MHz, DMSO-d₆) δ ppm 8.83 (br. s., 1H) 7.99-8.26 (m, 1H) 7.73-7.92 (m, 2H) 7.42 (br. s., 1H) 7.21 (d, J=8.83 Hz, 1H) 3.94 (s, 3H) 1.20-1.36 (m, 9H); MS (ES) m/z 420, 422 [M+1]⁺.

### Example 64i

### 5-(3-Bromo-4-methoxyphenyl)-5-(2-(trifluoromethyl)pyridin-4-yl)-5H-pyrrolo[3,4-b]pyridin-7-amine

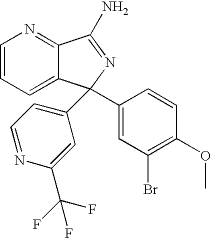

The title compound was synthesized as described for Example 5i in 66% yield starting from N-((3-bromo-4-methoxyphenyl)(2-cyanopyridin-3-yl)methylene)-2-methylpropane-2-sulfinamide (6.06 g, 14.42 mmol) and bromo-2-trifluoromethylpyridine (3.91 g, 17.30 mmol):

MS (ES) m/z 463, 465 [M+1]⁺.

### Example 65i

### 4-(7-Amino-5-(2-(trifluoromethyl)pyridin-4-yl)-5H-pyrrolo[3,4-b]pyridin-5-yl)-2-bromophenol

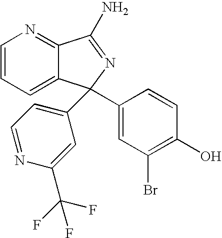

A solution of 5-(3-bromo-4-methoxyphenyl)-5-(2-(trifluoromethyl)pyridin-4-yl)-5H-pyrrolo[3,4-b]pyridin-7-amine (3.266 g, 7.05 mmol) in dry chloroform (50 mL) was cooled to 0° C. under argon atmosphere. Neat boron tribromide (2.000 mL, 21.15 mmol) was added dropwise over 2 min and the resulting solution was stirred at 0° C. for 15 mins and then at rt for 3 days. The reaction was quenched by water and the pH adjusted to >7 with aq sat NaHCO₃. The mixture was extracted with CHCl₃ three times and the combined organic layers were dried over MgSO₄, filtered, and the solvent was evaporated in vacuo. The crude product was purified on a silica gel column eluted with 0-10% 0.1M NH₃ (in MeOH) in DCM to give 4-(7-amino-5-(2-(trifluoromethyl)pyridin-4-yl)-5H-pyrrolo[3,4-b]pyridin-5-yl)-2-bromophenol (0.771 g, 24% yield):

¹H NMR (500 MHz, DMSO-d₆) δ ppm 10.28 (br. s., 1H) 8.68 (d, 2H) 8.20-8.51 (m, 1 H) 7.70-7.76 (m, 1H) 7.63-7.70 (m, 1H) 7.48-7.55 (m, 1H) 7.39 (d, 1H) 7.19 (dd, 1H) 7.01 (br. s., 2H) 6.86 (d, 1H); MS (ES+) m/z 449, 451 [M+1]⁺.

### Example 66i

### 4-Bromo-2-(difluoromethyl)-6-methylpyridine

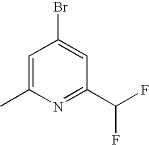

(4-Bromo-6-methylpyridin-2-yl)methanol (3 g, 14.85 mmol) was dissolved in chloroform (60 mL) under argon. Manganese(IV) oxide (15.19 g, 148.48 mmol) was added. The resulting mixture was stirred at reflux for 2 hours. The reaction mixture was filtered through celite and the filter was washed with chloroform (20 mL). The filtrate was cooled to 0° C. under argon and diethylaminosulphur trifluoride (3.41 mL, 27.84 mmol) was added. The reaction mixture was stirred over night while the temperature was allowed to reach ambient temperature. The reaction was quenched by addition of saturated aqueous sodium bicarbonate solution and was further diluted with dichloromethane. The organic layer was collected and the water phase was extracted three times with dichloromethane. The organic layers were combined, washed with brine, dried (MgSO₄), filtered and carefully concentrated at reduced pressure. Purification by silica gel column chromatography (0 to 20% diethyl ether in pentane) gave 1.00 g (30% yield) of the title compound:

¹H NMR (600 MHz, CDCl₃) δ ppm 7.62 (s, 1H) 7.46 (s, 1H) 6.57 (t, 1H) 2.58 (s, 3H); MS (EI) m/z 221, 223 [M]⁺.

### Example 67i

### 5-(3-Bromophenyl)-5-(2-(difluoromethyl)-6-methylpyridin-4-yl)-5H-pyrrolo[3,4-b]pyridin-7-amine

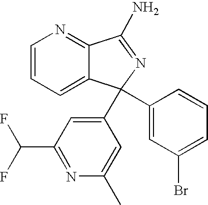

Butyllithium (0.666 mL, 1.67 mmol) was added to 4-bromo-2-(difluoromethyl)-6-methylpyridine (313 mg, 1.41 mmol) in THF (7 mL) at −78° C. under nitrogen atmosphere. The reaction was stirred for 30 min before N-((3-bromophenyl)(2-cyanopyridin-3-yl)methylene)-2-methylpropane-2-sulfinamide (500 mg, 1.28 mmol) in THF (3 mL) was added. The reaction was kept at −78° C. for 1 hour and then allowed to reach room temp. MeOH (5 mL) and hydrochloric acid in diethylether (3.84 mL, 3.84 mmol) were added. The reaction was stirred another two hours and then quenched with water and NaHCO₃ (sat) and extracted with EtOAc. The organics were collected, concentrated and purified using preparative HPLC to give the title compound 60 mg (11% yield):

MS (ES+) m/z 429, 431 [M+1]⁺.

### Example 1

### 5-(3′-Chlorobiphenyl-3-yl)-5-(2-(trifluoromethyl)pyridin-4-yl)-5H-pyrrolo[3,4-b]pyridin-7-amine

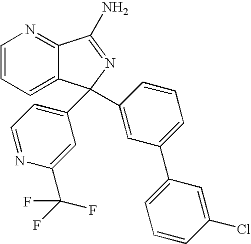

3-Chlorophenylboronic acid (61.0 mg, 0.39 mmol), 5-(3-bromophenyl)-5-(2-(trifluoromethyl)pyridin-4-yl)-5H-pyrrolo[3,4-b]pyridin-7-amine (130 mg, 0.30 mmol), Cesium carbonate (293 mg, 0.90 mmol) and dichloro[1,1′-bis(diphenylphosphino)ferrocene]palladium (II) dichloromethane adduct (12.25 mg, 0.02 mmol) were mixed in DME:EtOH:water (6:3:1) (3 mL) and heated in a microwave reactor for 20 min at 150° C. The mixture was filtered and purified with preparative HPLC to give 0.039 g (28% yield) of the title compound:

¹H NMR (500 MHz, DMSO-d₆) δ ppm 7.10 (br. s., 2H) 7.41-7.57 (m, 6H) 7.58-7.63 (m, 3H) 7.72-7.79 (m, 2H) 8.55-8.59 (m, 1H) 8.68-8.72 (m, 2H); MS (ES) m/z 463 [M−1]⁻

### Example 2

### 5-(3-(pyrimidin-5-yl)phenyl)-5-(2-(trifluoromethyl)pyridin-4-yl)-5H-pyrrolo[3,4-b]pyridin-7-amine

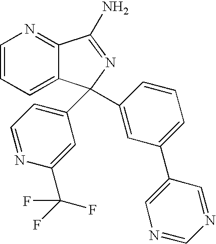

The title compound was synthesized as described for Example 1 in 48% yield starting from 5-(3-bromophenyl)-5-(2-(trifluoromethyl)pyridin-4-yl)-5H-pyrrolo[3,4-b]pyridin-7-amine (95 mg, 0.22 mmol) and pyrimidin-5-ylboronic acid (32.6 mg, 0.26 mmol).

¹H NMR (400 MHz, DMSO-d₆) δ ppm 7.07 (br. s., 2H) 7.46-7.59 (m, 3H) 7.69-7.79 (m, 4H) 8.58-8.63 (m, 1H) 8.68-8.73 (m, 2H) 9.05 (s, 2H) 9.18 (s, 1H); MS (ES) m/z 433 [M+1]⁺

### Example 3

### 5-(3-(Pyridin-3-yl)phenyl)-5-(2-(trifluoromethyl)pyridin-4-yl)-5H-pyrrolo[3,4-b]pyridin-7-amine

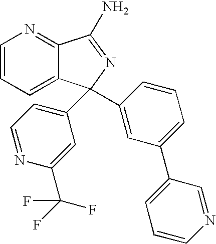

The title compound was synthesized as described for Example 5i in 53% yield starting from 5-(3-bromophenyl)-5-(2-(trifluoromethyl)pyridin-4-yl)-5H-pyrrolo[3,4-b]pyridin-7-amine (95 mg, 0.22 mmol) and pyridin-3-ylboronic acid (32.3 mg, 0.26 mmol.

¹H NMR (500 MHz, DMSO-d₆) δ ppm 7.09 (br. s., 2H) 7.44-7.49 (m, 3H) 7.52-7.58 (m, 1H) 7.60-7.66 (m, 2H) 7.73-7.80 (m, 2H) 7.94-7.99 (m, 1H) 8.54-8.61 (m, 2H) 8.67-8.72 (m, 2H) 8.77-8.80 (m, 1H); MS (ES+) m/z 432 [M+1]⁺.

### Example 4

### 5-(2,6-Dimethylpyridin-4-yl)-5-(3-(pyrimidin-5-yl)phenyl)-5H-pyrrolo[3,4-b]pyridin-7-amine

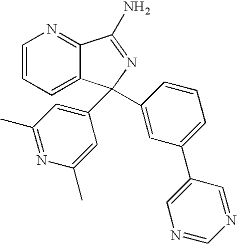

The title compound was synthesized as described for Example 1 in 36% yield starting from 3-(7-(tert-butoxycarbonylamino)-5-(2,6-dimethylpyridin-4-yl)-5H-pyrrolo[3,4-b]pyridin-5-yl)phenyl trifluoromethanesulfonate (230 mg, 0.41 mmol) and pyrimidin-5-ylboronic acid (60.8 mg, 0.49 mmol).

¹H NMR (500 MHz, DMSO-d₆) δ ppm 1.91 (s, acetate) 2.34 (s, 6H) 6.91 (br. s., 2H) 7.03 (s, 2H) 7.44-7.57 (m, 3H) 7.64-7.71 (m, 2H) 8.49-8.54 (m, 1H) 8.61-8.69 (m, 1H) 9.04 (s, 2H) 9.18 (s, 1H); MS (ES) m/z 393 [M+1]⁺.

### Example 5

### 5-(3-(7-Amino-5-(2,6-dimethylpyridin-4-yl)-5H-pyrrolo[3,4-b]pyridin-5-yl)phenyl)nicotinonitrile

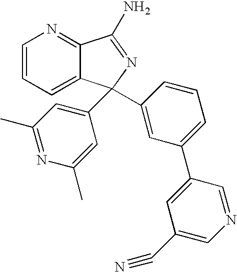

The title compound was synthesized as described for Example 5i in 41% yield starting from 3-(7-(tert-butoxycarbonylamino)-5-(2,6-dimethylpyridin-4-yl)-5H-pyrrolo[3,4-b]pyridin-5-yl)phenyl trifluoromethanesulfonate (230 mg, 0.41 mmol) and 5-cyanopyridin-3-ylboronic acid (72.6 mg, 0.49 mmol).

¹H NMR (500 MHz, DMSO-d₆) δ ppm 1.91 (s, acetate) 2.34 (s, 6H) 6.90 (br. s., 2H) 7.01 (s, 2H) 7.47 (t, 1H) 7.50-7.55 (m, 2H) 7.64-7.70 (m, 2H) 8.50-8.54 (m, 1H) 8.55-8.59 (m, 1H) 8.63-8.69 (m, 1H) 8.98-9.01 (m, 1H) 9.05-9.09 (m, 1H). MS (ES) m/z 417 [M+1]⁺

### Example 6

### 5-(3,5-Difluoro-4-methoxyphenyl)-5-(4-fluoro-3-(pyrimidin-5-yl)phenyl)-5H-pyrrolo[3,4-b]pyridin-7-amine

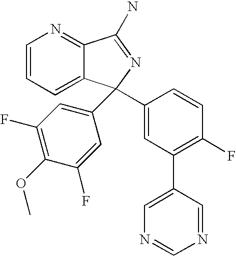

Pyrimidine-5-boronic acid (83 mg, 0.67 mmol), 5-(3-bromo-4-fluorophenyl)-5-(3,5-difluoro-4-methoxyphenyl)-5H-pyrrolo[3,4-b]pyridin-7-amine (250 mg, 0.56 mmol), cesium carbonate (545 mg, 1.67 mmol) and dichloro[1,1′-bis(diphenylphosphino)ferrocene]palladium (II) dichloromethane adduct (46 mg, 0.06 mmol) were dissolved in DME:EtOH:Water (6:3:1) (5 mL) and irradiated in a microwave oven for 20 min at 150° C. EtOAc, water and brine was added and the organic phase was collected, dried, and filtered. The product was purified with preparative HPLC. The pure fractions were pooled and concentrated in vacuo. This gave 12 mg (5% yield) of the title product:

¹H NMR (500 MHz, CDCL3) δ ppm 9.13 (s, 1H) 8.79 (br. s., 2H) 8.61 (d, 1H) 7.81 (d, 1 H) 7.26-7.42 (m, 3H) 7.09 (t, 1H) 6.73-6.83 (m, 2H) 3.89 (s, 3H); MS (ES) m/z 448 [M+1]⁺

### Example 7

### 5-(3-Chloro-4-methoxyphenyl)-5-(3-(pyrimidin-5-yl)phenyl)-5H-pyrrolo[3,4-b]pyridin-7-amine

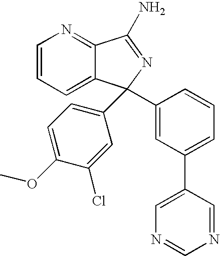

The title compound was synthesized as described for Example 1 in 30% yield starting from 5-(3-bromophenyl)-5-(3-chloro-4-methoxyphenyl)-5H-pyrrolo[3,4-b]pyridin-7-amine (200 mg, 0.47 mmol) and pyrimidin-5-ylboronic acid (69.4 mg, 0.56 mmol):

¹H NMR (500 MHz, DMSO-d₆) δ ppm 9.18 (s, 1H) 9.03 (s, 2H) 8.60-8.69 (m, 1H) 8.48 (dd, 1H) 7.62-7.71 (m, 2H) 7.43-7.56 (m, 3H) 7.34 (d, 1H) 7.30 (dd, 1H) 7.05 (d, 1H) 6.86 (br. s., 2H) 3.80 (s, 3H); MS (ES+) m/z 428, 430 [M+1]⁺.

### Example 8

### 5-(3-Chloro-4-methoxyphenyl)-5-(3-(pyrazin-2-yl)phenyl)-5H-pyrrolo[3,4-b]pyridin-7-amine

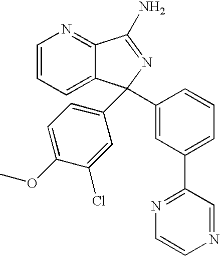

5-(3-Bromophenyl)-5-(3-chloro-4-methoxyphenyl)-5H-pyrrolo[3,4-b]pyridin-7-amine (145 mg, 0.34 mmol), 2-tributylstannylpyrazine (137 mg, 0.37 mmol), tetrakis(triphenylphosphine)palladium(0) (39.1 mg, 0.03 mmol) and DMF (2 mL) were added into a vial and heated in a microwave reactor at 150° C. for 15 min. When cooled to rt the mixture was filtered, and purified by preparative HPLC to give 40 mg (28% yield) of the title compound:

¹H NMR (500 MHz, DMSO-d₆) δ ppm 9.17 (d, 1H) 8.69 (dd, 1H) 8.65 (dd, 1H) 8.59 (d, 1H) 8.34 (dd, 1H) 8.11 (t, 1H) 7.99 (dt, 1H) 7.42-7.52 (m, 3H) 7.34 (d, 1H) 7.29 (dd, 1H) 7.06 (d, 1H) 6.88 (br. s., 2H) 3.80 (s, 3H); MS (ES+) m/z 428, 430 [M+1]⁺.

### Example 9

### 5-(3-Cyclopropyl-4-(difluoromethoxy)phenyl)-5-(3-(pyrimidin-5-yl)phenyl)-5H-pyrrolo[3,4-b]pyridin-7-amine acetic acid

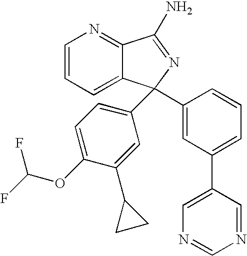

The title compound was synthesized as described for Example 1 in 12% yield starting from 5-(3-bromophenyl)-5-(3-cyclopropyl-4-(difluoromethoxy)phenyl)-5H-pyrrolo[3,4-b]pyridin-7-amine (150 mg, 0.32 mmol) and pyrimidine-5-boronic acid (43.5 mg, 0.35 mmol):

¹H NMR (500 MHz, DMSO-d₆) δ ppm 9.17 (s, 1H) 9.01 (s, 2H) 8.63 (dd, 1H) 8.44 (dd, 1H) 7.62-7.67 (m, 2H) 7.42-7.52 (m, 3H) 7.22 (dd, 1H) 7.13 (t, 1H) 7.04 (d, 1H) 6.95 (d, 1H) 6.82 (br. s., 2H) 1.97-2.04 (m, 1H) 1.91 (s, 3H) 0.90 (dd, 2H) 0.45-0.53 (m, 2H); MS (ES+) m/z 470 [M+1]⁺.

### Example 10

### 3-Chloro-5-(2-methylpyridin-4-yl)-5-(3-(pyrimidin-5-yl)phenyl)-5H-pyrrolo[3,4-b]pyridin-7-amine

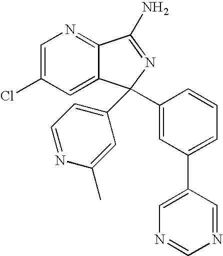

The title compound was synthesized as described for Example 1 in 8% yield starting from 5-(3-bromophenyl)-3-chloro-5-(2-methylpyridin-4-yl)-5H-pyrrolo[3,4-b]pyridin-7-amine (530 mg, 1.28 mmol) and pyrimidine-5-boronic acid (190 mg, 1.54 mmol):

¹H NMR (400 MHz, DMSO-d₆) δ ppm 9.18 (s, 1H) 9.07 (s, 2H) 8.78 (d, 1H) 8.72 (d, 1 H) 8.34 (d, 1H) 7.65-7.73 (m, 2H) 7.51-7.57 (m, 1H) 7.43-7.51 (m, 1H) 7.23-7.27 (m, 1H) 7.19 (dd, 1H) 7.02 (br. s., 2H) 2.40 (s, 3H): MS (ES+) m/z 413, 415 [M+1]⁺.

### Example 11

### 5-(4-Methoxyphenyl)-3-methyl-5-(3-(pyrimidin-5-yl)phenyl)-5H-pyrrolo[3,4-b]pyridin-7-amine

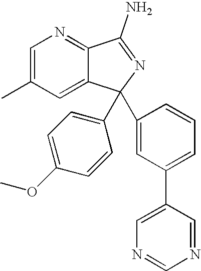

The title compound was synthesized as described for Example 1 in 58% yield starting from 5-(3-bromophenyl)-5-(4-methoxyphenyl)-3-methyl-5H-pyrrolo[3,4-b]pyridin-7-amine (1.9 g, 4.65 mmol) and pyrimidine-5-boronic acid (0.692 g, 5.58 mmol):

¹H NMR (400 MHz, DMSO-d₆) δ ppm 9.17 (s, 1H) 9.01 (s, 2H) 8.45 (d, 1H) 8.19 (d, 1 H) 7.60-7.67 (m, 2H) 7.47-7.52 (m, 1H) 7.40-7.47 (m, 1H) 7.22-7.29 (m, 2H) 6.79-6.86 (m, 2H) 6.70 (br. s., 2H) 3.69 (s, 3H) 2.42 (s, 3H) 1.90 (s, 3H); MS (ES+) m/z 408 [M+1]⁺.

### Example 12

### 5-(4-(Difluoromethoxy)-3,5-dimethylphenyl)-5-(3-(pyrimidin-5-yl)phenyl)-5H-pyrrolo[3,4-b]pyridin-7-amine

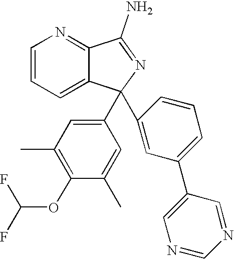

5-(3-Bromophenyl)-5-(4-(difluoromethoxy)-3,5-dimethylphenyl)-5H-pyrrolo[3,4-b]pyridin-7-amine (100 mg, 0.22 mmol), pyrimidin-5-ylboronic acid (35.1 mg, 0.28 mmol), [1,1-bis(diphenylphosphino)ferrocene]dichloropalladium(II) (17.82 mg, 0.02 mmol), potassium carbonate 2 M (aq) (0.327 mL, 0.65 mmol) and DMF (2 mL) were microwaved for 15 min at 150° C. The resulting mixture was diluted with brine and EtOAc and the phases separated. The aq phase was extracted with EtOAc (×2), the organics combined, dried (Na₂SO₄), filtered and concentrated. Purification was achieved by preparative chromatography to give the title compound (55 mg, 55% yield):

¹H NMR (600 MHz, DMSO-d₆) δ ppm 9.17 (s, 1H), 9.02 (s, 2H), 8.63 (dd, 1H), 8.48 (dd, 1H), 7.67 (s, 1H), 7.64 (d, 1H), 7.53 (d, 1H), 7.43-7.51 (m, 2H), 7.16 (s, 2H), 6.75-7.02 (t, 1H), 6.78 (br s, 2H), 2.16 (s, 6H); MS (ES+) m/z 458 [M+1]⁺.

### Example 13

### 5-(4-(Difluoromethoxy)-3,5-dimethylphenyl)-5-(3-(pyrazin-2-yl)phenyl)-5H-pyrrolo[3,4-b]pyridin-7-amine

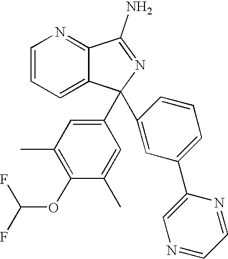

5-(3-Bromophenyl)-5-(4-(difluoromethoxy)-3,5-dimethylphenyl)-5H-pyrrolo[3,4-b]pyridin-7-amine (127 mg, 0.28 mmol), 2-(tributylstannyl)pyrazine (0.114 mL, 0.36 mmol), palladiumtetrakis (32.0 mg, 0.03 mmol) and DMF (2 mL) were microwaved for 15 min at 150° C. The mixture was diluted with brine and EtOAc, and then the passes were separated. The aq phase was extracted with EtOAc (×2), the organics combined, dried (Na₂SO₄), filtered and concentrated. Purification by preparative chromatography gave the title compound (50.5 mg, 37.5% yield):

¹H NMR (600 MHz, DMSO-d₆) δ ppm 9.16 (d, 1H), 8.66-8.70 (m, 1H), 8.64 (d, 1H), 8.59 (d, 1H), 8.36 (d, 1H), 8.12 (s, 1H), 7.97 (d, 1H), 7.43-7.52 (m, 3H), 7.15 (s, 2H), 6.89 (t, 1H), 6.80 (br s, 2H), 2.17 (s, 6H); MS (ES+) m/z 458 [M+1]⁺.

### Example 14

### 5-(4-Fluoro-3,5-dimethylphenyl)-5-(3-(pyrimidin-5-yl)phenyl)-5H-pyrrolo[3,4-b]pyridin-7-amine

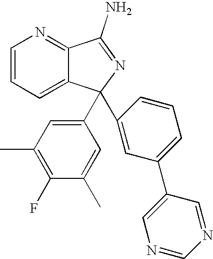

5-(3-Bromophenyl)-5-(4-fluoro-3,5-dimethylphenyl)-5H-pyrrolo[3,4-b]pyridin-7-amine (170 mg, 0.41 mmol), pyrimidin-5-ylboronic acid (66.7 mg, 0.54 mmol), [1,1-bis(diphenylphosphino)ferrocene]dichloropalladium(II) (30.3 mg, 0.04 mmol), potassium carbonate 2 M (aq) (0.622 mL, 1.24 mmol) and DMF (3 mL) were microwaved for 15 min at 150° C. The mixture was diluted with brine and EtOAc, and the phases were separated. The aqueous phase was extracted with EtOAc (×2), the organics combined, dried (Na₂SO₄), filtered and concentrated. Purification by preparative chromatography gave the title compound as the trifluoroacetate salt (116 mg, 52% yield):

¹H NMR (600 MHz, DMSO-d₆) δ ppm 12.25 (s, 1H), 10.28 (br. s., 1H), 9.90 (br. s., 1H), 9.20 (s, 1H), 9.11 (s, 2H), 8.95 (dd, 1H), 8.56 (dd, 1H), 7.88 (dd, 1H), 7.85 (m, 1H), 7.70 (t, 1H), 7.59 (t, 1H), 7.41-7.46 (m, 1H), 7.04 (d, 2H), 2.17 (s, 6H); MS (ES+) m/z 410 [M+1]⁺.

### Example 15

### 5-(3-Fluoro-4-methoxy-5-methylphenyl)-5-(3-(pyrazin-2-yl)phenyl)-5H-pyrrolo[3,4-b]pyridin-7-amine trifluoroacetic acid salt

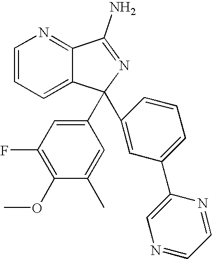

5-(3-Bromophenyl)-5-(3-fluoro-4-methoxy-5-methylphenyl)-5H-pyrrolo[3,4-b]pyridin-7-amine (79 mg, 0.19 mmol), 2-(tributylstannyl)pyrazine (0.076 mL, 0.24 mmol), palladiumtetrakis (21.42 mg, 0.02 mmol) and DMF (2 mL) were microwaved for 15 min at 150° C. The mixture was diluted with brine and EtOAc, and the phases separated. The aq phase was extracted with EtOAc (×2), the organics combined, dried (Na₂SO₄), filtered and concentrated. Purification by preparative chromatography gave the title compound as the trifluoroacetate salt (13.4 mg, 13% yield):

¹H NMR (600 MHz, DMSO-d₆) δ ppm 12.25 (br. s., 1H), 10.32 (br. s., 1H), 9.92 (br. s., 1 H), 9.24 (s, 1H), 8.96 (br. s., 1H), 8.71 (dd, 1H), 8.64 (d, 1H), 8.54 (d, 1H), 8.17 (d, 1H), 8.05 (s, 1H), 7.89 (br. s., 1H), 7.60 (t, 1H), 7.41-7.50 (m, 1H), 7.05 (dd, 1H), 7.02 (s, 1H), 3.83 (m, 3H), 2.19 (s, 3H); MS (ES+) m/z 426 [M+1]⁺.

### Example 16

### 5-(3-(Pyrimidin-5-yl)phenyl)-5-(2-(2,2,2-trifluoroethoxy)pyridin-4-yl)-5H-pyrrolo[3,4-b]pyridin-7-amine 0.2 acetic acid

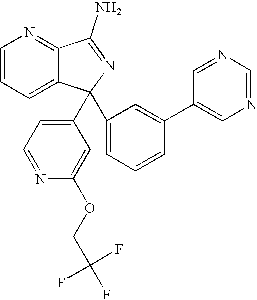

5-(3-Bromophenyl)-5-(2-(2,2,2-trifluoroethoxy)pyridin-4-yl)-5H-pyrrolo[3,4-b]pyridin-7-amine (159.4 mg, 0.34 mmol), 5-pyrimidinylboronic acid (53.3 mg, 0.43 mmol), [1,1′-bis(diphenylphosphino)ferrocene]palladium(II) chloride (16.33 mg, 0.02 mmol), cesium carbonate (0.080 mL, 1.00 mmol) and DME:EtOH:water (6:3:1) (4.00 mL) were put in a microwave vial and heated at 150° C. in a microwave reactor for 20 min. The reaction mixture was filtered through a syringe filter and purified by prep-HPLC. The desired fractions were pooled and freeze dried over night to give 5-(3-(pyrimidin-5-yl)phenyl)-5-(2-(2,2,2-trifluoroethoxy)pyridin-4-yl)-5H-pyrrolo[3,4-b]pyridin-7-amine (66.4 mg, 41% yield):

¹H NMR (400 MHz, DMSO-d₆) δ ppm 9.18 (s, 1H) 9.05 (s, 2H) 8.67 (dd, 1H) 8.58 (dd, 1H) 8.09 (d, 1H) 7.72 (t, 1H) 7.69 (dt, 1H) 7.55-7.59 (m, 1H) 7.46-7.55 (m, 2H) 7.12 (dd, 1H) 6.98 (br. s., 2H) 6.85 (dd, 1H) 4.92 (q, 2H) 1.86 (s, 0.57H); MS (ES+) m/z 463 [M+1]⁺.

### Example 17

### 5-(2-(2,2-Difluorovinyloxy)pyridin-4-yl)-5-(3-(pyrimidin-5-yl)phenyl)-5H-pyrrolo[3,4-b]pyridin-7-amine 0.2 acetic acid

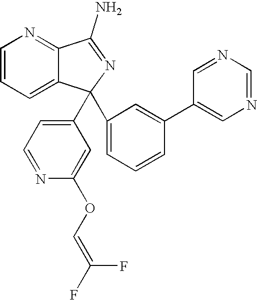

5-(3-Bromophenyl)-5-(2-(2,2-difluorovinyloxy)pyridin-4-yl)-5H-pyrrolo[3,4-b]pyridin-7-amine bis(2,2,2-trifluoroacetic acid) (63.7 mg, 0.09 mmol), 5-pyrimidinylboronic acid (15.28 mg, 0.12 mmol), [1,1′-bis(diphenylphosphino)ferrocene]palladium(II) chloride (3.87 mg, 4.74 μmol), cesium carbonate (0.023 mL, 0.28 mmol) and DME:EtOH:water (6:3:1) (2.00 mL) were put in a microwave vial and heated at 150° C. in a microwave reactor for 20 min. The reaction mixture was filtered through a syringe filter and purified by prep-HPLC. The desired fractions were pooled and freeze dried over night to give 5-(2-(2,2-difluorovinyloxy)pyridin-4-yl)-5-(3-(pyrimidin-5-yl)phenyl)-5H-pyrrolo[3,4-b]pyridin-7-amine (26 mg, 60% yield):

¹H NMR (400 MHz, DMSO-d₆) δ ppm 9.18 (s, 1H) 9.05 (s, 2H) 8.67 (dd, 1H) 8.58 (dd, 1H) 8.12 (dd, 1H) 7.72 (t, 1H) 7.69 (dt, 1H) 7.43-7.59 (m, 3H) 7.15-7.25 (m, 2H) 6.87-7.05 (m, 3H) 1.83 (s, 0.62H); MS (ES+) m/z 443 [M+1]⁺.

### Example 18

### 5-(4-(Difluoromethoxy)-3-fluorophenyl)-5-(3-(pyrimidin-5-yl)phenyl)-5H-pyrrolo[3,4-b]pyridin-7-amine

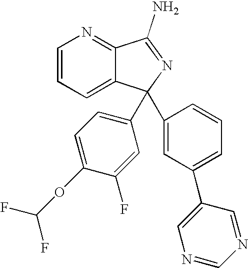

5-(3-bromophenyl)-5-(4-(difluoromethoxy)-3-fluorophenyl)-5H-pyrrolo[3,4-b]pyridin-7-amine (35 mg, 0.08 mmol), pyrimidine-5-boronic acid (11.61 mg, 0.09 mmol), cesium carbonate (76 mg, 0.23 mmol) and dichloro[1,1′-bis(diphenylphosphino)ferrocene]palladium (II) dichloromethane adduct (6.38 mg, 7.81 μmol) were dissolved in 1,2-dimethoxyethane, water and ethanol (6:1:3, 5 mL) and irradiated in a microwave oven for 20 min at 150° C. EtOAc, water and brine was added, and the organic phase was separated, dried and filtered. The product was purified by preparative HPLC. to give 10 mg (28% yield) of the title product:

¹H NMR (400 MHz, DMSO-d₆) δ ppm 7.00 (br. s., 2H) 7.19 (t, 1H) 7.26-7.30 (m, 2H) 7.34 (d, 1H) 7.44-7.57 (m, 3H) 7.64-7.72 (m, 2H) 8.53 (dd, 1H) 8.65 (dd, 1H) 9.04 (s, 2H) 9.18 (s, 1H); MS (ES) m/z 448 [M+1]⁺.

### Example 19

### 5-(3-(4-methoxypyridin-2-yl)phenyl)-5-(2-(trifluoromethyl)pyridin-4-yl)-5H-pyrrolo[3,4-b]pyridin-7-amine

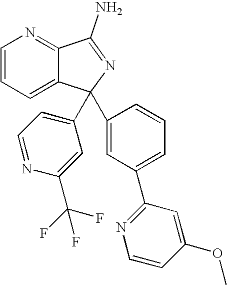

5-(3-bromophenyl)-5-(2-(trifluoromethyl)pyridin-4-yl)-5H-pyrrolo[3,4-b]pyridin-7-amine (220 mg, 0.51 mmol), 4-methoxy-2-(tributylstannyl)pyridine (212 mg, 0.53 mmol), tetrakis(triphenylphosphine)palladium(0) (58.7 mg, 0.05 mmol) and DMF (4 mL) were put in a microwave vial and irradiated in a microwave reactor at 150° C. for 20 min. When cooled to ambient temperature the mixture was filtered and the product was purified by preparative HPLC to give 5-(3-(4-methoxypyridin-2-yl)phenyl)-5-(2-(trifluoromethyl)pyridin-4-yl)-5H-pyrrolo[3,4-b]pyridin-7-amine (35.0 mg, 8% yield):

¹H NMR (500 MHz, DMSO-d₆) δ ppm 3.94 (s, 3H) 7.12 (br. s., 1H) 7.41 (d, 1H) 7.56 (dl H) 7.60 (t, 1H) 7.76 (dt, 1H) 7.88-7.90 (m, 1H) 7.92-7.97 (m, 2H) 8.10 (d, 1H) 8.53 (d, 1H) 8.63 (dd, 1H) 8.84 (d, 1H) 9.01 (dd, 1H) 10.16 (br. s., 1H) 10.49 (br. s., 1H) 12.48 (br. s., 1H); MS (ES) m/z 462 [M+1]⁺.

### Example 20

### 5-(2-(Difluoromethyl)-6-methylpyridin-4-yl)-5-(3-(pyrimidin-5-yl)phenyl)-5H-pyrrolo[3,4-b]pyridin-7-amine

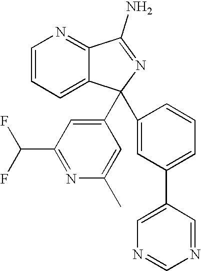

5-(3-Bromophenyl)-5-(2-(difluoromethyl)-6-methylpyridin-4-yl)-5H-pyrrolo[3,4-b]pyridin-7-amine (60 mg, 0.14 mmol), pyrimidin-5-ylboronic acid (19.05 mg, 0.15 mmol) and (1,1′-bis(diphenylphosphino)ferrocene)-dichloropalladium(II) (11.50 mg, 0.01 mmol) were mixed in THF (3 mL). Aqueous sodium carbonate (2 M, 0.210 mL, 0.42 mmol) was added and the mixture was run in a microwave for 40 min at 140° C. The mixture was filtered and purified by preparative HPLC to give the title compound (20 mg, 33% yield):

¹H NMR (400 MHz, DMSO-d₆) δ ppm 9.18 (s, 1H) 9.05 (s, 2H) 8.64-8.71 (m, 1H) 8.53-8.60 (m, 1H) 7.65-7.74 (m, 2H) 7.51-7.57 (m, 2H) 7.50 (d, 1H) 7.45-7.47 (m, 1H) 7.42 (s, 1H) 7.01 (br. s., 2H) 6.99-6.72 (t, 1H) 2.47 (s, 3H), MS (ES+) m/z 429 [M+1]⁺.

### Example 21

### 5-(3-(5-Chloropyridin-3-yl)phenyl)-5-(2-(trifluoromethyl)pyridin-4-yl)-5H-pyrrolo[3,4-b]pyridin-7-amine

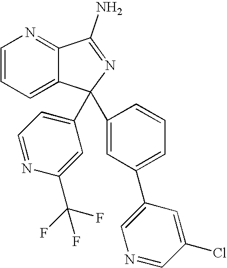

The title compound was synthesized as described for Example 18 in 22% yield, starting from 5-(3-bromophenyl)-5-(2-(trifluoromethyl)pyridin-4-yl)-5H-pyrrolo[3,4-b]pyridin-7-amine (230 mg, 0.53 mmol), and 5-chloropyridin-3-ylboronic acid (100 mg, 0.63 mmol):

¹H NMR (500 MHz, DMSO-d₆) δ ppm 7.04 (br. s., 2H) 7.47 (t, 1H) 7.52-7.58 (m, 2H) 7.66-7.71 (m, 2H) 7.74 (dd, 1H) 7.75-7.77 (m, 1H) 8.16 (t, 1H) 8.59 (dd, 1H) 8.62 (d, 1H) 8.70 (dd, 2H) 8.76 (d, 1H); MS (ES) m/z 466 [M+1]⁺.

### Example 22

### 2-(3-(7-Amino-5-(2-(trifluoromethyl)pyridin-4-yl)-5H-pyrrolo[3,4-b]pyridin-5-yl)phenyl)isonicotinonitrile

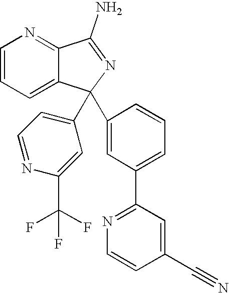

The title compound was synthesized as described for Example 19 in 19% yield, starting from 5-(3-bromophenyl)-5-(2-(trifluoromethyl)pyridin-4-yl)-5H-pyrrolo[3,4-b]pyridin-7-amine (250 mg, 0.58 mmol), and 2-(trimethylstannyl)isonicotinonitrile (231 mg, 0.87 mmol):

¹H NMR (500 MHz, DMSO-d₆) δ ppm 8.81-8.94 (m, 1H) 8.64-8.75 (m, 2H) 8.51 (d, 1 H) 8.40 (s, 1H) 8.17 (s, 1H) 8.03 (d, 1H) 7.63-7.87 (m, 3H) 7.34-7.62 (m, 3H) 7.05 (br. s., 2H); MS (ES) m/z 457 [M+1]⁺.

### Example 23

### 5-(3-(difluoro methyl)-4-methoxyphenyl)-5-(3-(pyrazin-2-yl)phenyl)-5H-pyrrolo[3,4-b]pyridin-7-amine

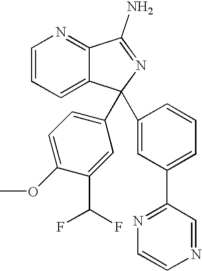

The title compound was synthesized as described for Example 19 in 24% yield, starting from 5-(3-bromophenyl)-5-(3-(difluoromethyl)-4-methoxyphenyl)-5H-pyrrolo[3,4-b]pyridin-7-amine (300 mg, 0.68 mmol), and 2-tributylstannylpyrazine (374 mg, 1.01 mmol):

¹H NMR (500 MHz, DMSO-d₆) δ ppm 9.16 (d, 1H) 8.67-8.69 (m, 1H) 8.65 (dd, 1H) 8.59 (d, 1H) 8.30 (dd, 1H) 8.12 (s, 1H) 7.98 (dt, 1H) 7.41-7.56 (m, 5H) 7.06 (d, 1H) 7.01 (t, 1H) 6.90 (br. s., 2H) 3.80 (s, 3H); MS (ES) m/z 444 [M+1]⁺.

### Example 24

### 5-(3-(Difluoromethyl)-4-methoxyphenyl)-5-(3-(pyrimidin-5-yl)phenyl)-5H-pyrrolo[3,4-b]pyridin-7-amine

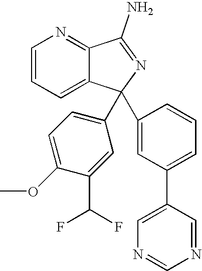

The title compound was synthesized as described for Example 18 in 29% yield, starting from 5-(3-bromophenyl)-5-(3-(difluoromethyl)-4-methoxyphenyl)-5H-pyrrolo[3,4-b]pyridin-7-amine (300 mg, 0.68 mmol), and pyrimidin-5-ylboronic acid (100 mg, 0.81 mmol), cesium carbonate (660 mg, 2.03 mmol) and dichloro[1,1′-s bis(diphenylphosphino)ferrocene]palladium (II) dichloromethane adduct (55 mg, 0.07 mmol):

¹H NMR (500 MHz, DMSO-d₆) δ ppm 9.17 (d, 1H) 9.02 (s, 2H) 8.56-8.70 (m, 1H) 8.43 (d, 1H) 7.59-7.70 (m, 2H) 7.36-7.56 (m, 5H) 7.07 (d, 1H) 7.01 (t, 1H) 6.81 (br. s., 2H) 3.80 (s, 3H); MS (ES) m/z 444 [M+1]⁺.

### Example 25

### Separation of 5-(3-(Pyrimidin-5-yl)phenyl)-5-(2-(trifluoromethyl)pyridin-4-yl)-5H-pyrrolo[3,4-b]pyridin-7-amine

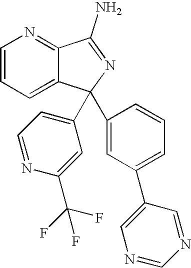

The enantiomers of 5-(3-(pyrimidin-5-yl)phenyl)-5-(2-(trifluoromethyl)pyridin-4-yl)-5H-pyrrolo[3,4-b]pyridin-7-amine (100 mg, 0.23 mmol) were separated by chromatography (Berger Multigram II system, Chiralpak AD; 21.2*250 mm, Mobilephase: 15% EtOH+0.1% DEA; 85% CO2 Flow: 50 ml/min) and the two isomers were collected and concentrated in vacuo. Isomer 1, 29 mg (29% yield) with unknown absolute configuration: ¹H NMR (500 MHz, DMSO-d₆) δ ppm 9.18 (s, 1H) 9.06 (s, 2H) 8.69 (d, 2H) 8.62 (dd, 1H) 7.66-7.83 (m, 4H) 7.53-7.58 (m, 2H) 7.51 (t, 1H) 7.08 (br. s., 2H); MS (ES) m/z 433 [M+1]⁺.

Isomer 2, 34 mg (34% yield) with unknown absolute configuration:

¹H NMR (500 MHz, DMSO-d₆) δ ppm 9.18 (s, 1H) 9.06 (s, 2H) 8.69 (d, 2H) 8.62 (dd, 1H) 7.66-7.83 (m, 4H) 7.53-7.58 (m, 2H) 7.51 (t, 1H) 7.08 (br. s., 2H); MS (ES) m/z 433 [M+1]⁺.

### Example 26

### 5-(4-(Fluoromethoxy)-3,5-dimethylphenyl)-5-(3-(pyrimidin-5-yl)phenyl)-5H-pyrrolo[3,4-b]pyridin-7-amine

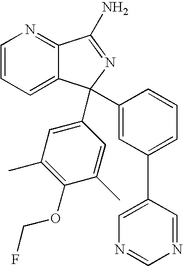

5-(3-Bromophenyl)-5-(4-(fluoromethoxy)-3,5-dimethylphenyl)-5H-pyrrolo[3,4-b]pyridin-7-amine (90 mg, 0.20 mmol), pyrimidin-5-ylboronic acid (27.9 mg, 0.22 mmol) and (1,1′-bis(diphenylphosphino)ferrocene)-dichloropalladium(II) (16.82 mg, 0.02 mmol) were mixed in THF (3.5 mL). Sodium carbonate (2M) (0.307 mL, 0.61 mmol) was added and the mixture was run in a microwave for 40 min at 140° C. The mixture was filtered and is purified by preparative HPLC to give the title compound (30 mg, 33% yield):

¹H NMR (500 MHz, CDCl₃) d ppm 9.18 (s, 1H) 8.88 (s, 2H) 8.60-8.69 (m, 1H) 7.93 (dd, 1H) 7.56 (s, 1H) 7.45-7.49 (m, 1H) 7.44 (d, 2H) 7.38 (dd, 1H) 6.98 (s, 2H) 5.58 (s, 2H) 5.47 (d, 1H) 2.21 (s, 6H); MS (ES+) m/z 440 [M+1]⁺.

### Example 27

### 5-(4-(Fluoromethoxy)-3,5-dimethylphenyl)-5-(3-(pyrazin-2-yl)phenyl)-5H-pyrrolo[3,4-b]pyridin-7-amine

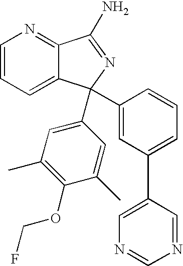

2-(Tributylstannyl)pyrazine (0.071 mL, 0.22 mmol), 5-(3-bromophenyl)-5-(4-(fluoromethoxy)-3,5-dimethylphenyl)-5H-pyrrolo[3,4-b]pyridin-7-amine (90 mg, 0.20 mmol) and tetrakis(triphenylphosphine)palladium(0) (23.62 mg, 0.02 mmol) were dissolved in toluene/metanol 9:1 (4 mL) and run in a microwave oven for 20 min at 130° C. 2-(Tributylstannyl)pyrazine (0.071 mL, 0.22 mmol) and tetrakis(triphenylphosphine)palladium(0) (23.62 mg, 0.02 mmol) were added and the mixture was run for 40 min at 130° C. in the microwave oven. The mixture was concentrated and the residue dissolved in DMF and purified by preparative HPLC. Residual triphenylphoshineoxide was removed using a porapak column, the product was eluated with 5% NH₃ in MeOH. The mixture was concentrated to give the title compound (15 mg, 17% yield):

¹H NMR (500 MHz, CDCl₃) δ ppm 8.95 (s, 1H) 8.56-8.70 (m, 2H) 8.48 (s, 1H) 7.94-8.02 (m, 2H) 7.92 (d, 1H) 7.41-7.55 (m, 2H) 7.33-7.41 (m, 1H) 6.98 (s, 2H) 5.57 (br. s., 1H) 5.46 (br. s., 1H) 2.11-2.28 (m, 6H); MS (ES+) m/z 440 [M+1]⁺.

### Example 28

### 5-(2-(3-Fluoropropoxy)pyridin-4-yl)-5-(3-(pyrimidin-5-yl)phenyl)-5H-pyrrolo[3,4-b]pyridin-7-amine

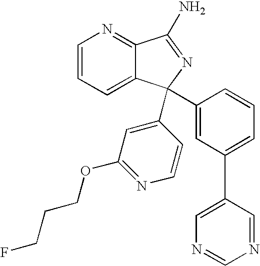

5-(3-bromophenyl)-5-(2-(3-fluoropropoxy)pyridin-4-yl)-5H-pyrrolo[3,4-b]pyridin-7-amine (450 mg, 1.02 mmol), pyrimidin-5-yl boronic acid (139 mg, 1.12 mmol) and (1,1′-bis(diphenylphosphino)ferrocene)-dichloropalladium(II) (84 mg, 0.10 mmol) were mixed in THF (3 mL). Sodium carbonate (2M) (1.530 mL, 3.06 mmol) was added and the mixture was run in a microwave for 40 min at 140° C. The mixture was filtered and purified by preparative HPLC to give the title compound (55 mg, 12% yield):

¹H NMR (400 MHz, CDCl₃) δ ppm 9.18 (s, 1H) 8.87 (s, 2H) 8.66 (dd, Hz, 1H) 8.07 (d, 1 H) 7.95 (dd, 1H) 7.53 (s, 1H) 7.48 (dd, 1H) 7.43-7.47 (m, 1H) 7.36-7.43 (m, 2H) 6.85 (dd, 1H) 6.70 (d, 1H) 4.65 (t, 1H) 4.53 (t, 1H) 4.40 (t, 2H) 2.04-2.24 (m, 2H); MS (ES+) m/z 441 [M+1]⁺.

### Example 29

### 5-(4-Difluoromethoxy-3,5-dimethyl-phenyl)-5-(2-pyrimidin-5-yl-pyridin-4-yl)-5H-pyrrolo[3,4-b]pyridin-7-ylamine

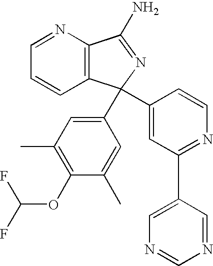

A mixture of 5-(2-chloro-pyridin-4-yl)-5-(4-difluoromethoxy-3,5-dimethyl-phenyl)-5H-pyrrolo[3,4-b]pyridin-7-ylamine (85 mg, 0.20 mmol), pyrimidine-5-boronic acid (76 mg, 0.60 mmol), Pd(PPh₃)₄ (24 mg, 0.002 mmol), K₂CO₃ (85 mg, 0.60 mmol) in a mixture of DME-water (7:1, 4 mL) was degassed using nitrogen for 15 minutes and then heated at 90° C. in a sealed tube for 17 h. The mixture was cooled to room temperature, diluted with EtOAc (20 mL) and was washed with saturated NaHCO₃ solution (10 mL), H₂O (10 mL), dried over Na₂SO₄, filtered and concentrated under reduced pressure. The residue was purified by preparative HPLC to afford 35 mg (38% yield) of the title compound. ¹H NMR (400 MHz, CDCl₃) δ ppm 9.27 (s, 2H) 9.24 (s, 1H) 8.72 (d, 1H) 8.68 (d, 1H) 7.96 (d, 1H) 7.76 (s, 1H) 7.48 (dd, 1H) 7.29 (d, 1H) 6.96 (s, 2H) 6.31 (t, 1H) 5.43 (br.s, 2H) 2.24 (s, 6H); MS (ES+) m/z: 459.22, 460.22 [M+1]⁺.

### Example 30

### 5-(3-Cyclopropyl-4-difluoromethoxy-5-methyl-phenyl)-5-(2-pyrimidin-5-yl-pyridin-4-yl)-5H-pyrrolo[3,4-b]pyridin-7-ylamine

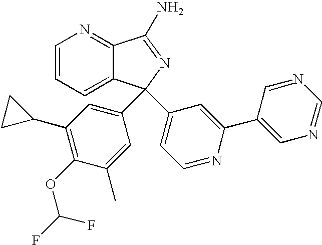

A mixture of 5-(2-chloro-pyridin-4-yl)-5-(3-cyclopropyl-4-difluoromethoxy-5-methyl-phenyl)-5H-pyrrolo[3,4-b]pyridin-7-ylamine (0.16 g, 0.36 mmol), pyrimidine-5-boronic acid (67.5 mg, 0.54 mmol), Pd(PPh₃)₄ (84 mg, 0.073 mmol), Na₂CO₃ (2M, 1 mL, 2 mmol) in DME (4 mL) was degassed using nitrogen for 15 minutes and then heated at 90° C. in a sealed tube for 16 hours. The mixture was cooled to room temperature, diluted with EtOAc (20 mL) and washed with saturated NaHCO₃ solution (10 mL), H₂O (10 mL), dried over Na₂SO₄ and concentrated under reduced pressure. The residue was purified by preparative HPLC to afford 80 mg (45% yield) of the title compound:

¹H NMR (400 MHz, CDCl₃) δ ppm 9.21-9.27 (m, 3H) 8.68 (d, 1H) 8.64 (d, 1H) 7.87-7.92 (m, 1H) 7.69 (s, 1H) 7.41 (dd, 1H) 7.22-7.25 (m, 1H) 6.94 (s, 1H) 6.67 (d, 1H) 6.44 (t, 1H) 5.48 (s, 2H) 2.26 (s, 3H) 2.03-2.12 (m, 1H) 0.95 (d, 2H) 0.50-0.59 (m, 2H); MS (ES+) m/z: 485.17 [M+1]⁺.

### Example 31

### 5-[3-Cyclopropyl-4-(difluoromethoxy)-5-methyl-phenyl]-5-phenyl-pyrrolo[3,4-b]pyridin-7-amine

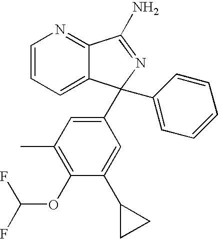

5-(3-Bromo-phenyl)-5-(3-cyclopropyl-4-difluoromethoxy-5-methyl-phenyl)-5H-pyrrolo[3,4-b]pyridin-7-ylamine (125 mg, 0.26 mmol) and palladium on charcoal (10 wt %, 12 mg, 0.03 mmol) were taken in MeOH (5 mL) and the mixture was stirred under H₂ atmosphere at room temperature overnight. The reaction was filtered through a pad of Celite and concentrated under reduced pressure. The residue was purified by preparative HPLC to afford 0.08 g (76% yield) of the title compound:

¹H NMR (400 MHz, CDCl₃) δ ppm 8.60 (d, 1H) 7.84 (d, 1H) 7.32 (dd, 1H) 7.30-7.24 (m, 5H) 6.96 (d, 1H) 6.67 (d, 1H) 6.42 (m, 1H) 5.30 (s, 2H) 2.23 (s, 3H) 1.96-2.12 (m, 1H) 0.91 (d, 2H) 0.42-0.64 (m, 2H); MS (ES+) m/z: 405.92 [M+1]⁺.

### Example 32

### 3-[7-Amino-5-(3-cyclopropyl-4-difluoromethoxy-5-methyl-phenyl)-5H-pyrrolo[3,4-b]pyridin-5-yl]-benzonitrile

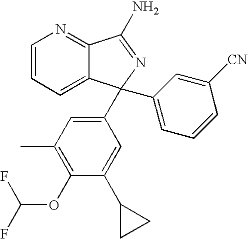

A mixture of 5-(3-bromo-phenyl)-5-(3-cyclopropyl-4-difluoromethoxy-5-methyl-phenyl)-5H-pyrrolo[3,4-b]pyridin-7-ylamine (195 mg, 0.40 mmol), zinc cyanide (47 mg, 0.40 mmol), Pd(PPh₃)₄ (23 mg, 20 μmol) in dry DMF (3 mL) was degassed and purged with nitrogen for 10 minutes and heated in a microwave reactor at 80° C. for 1 hour. The reaction mixture was diluted with EtOAc (10 mL), washed with water and brine, dried over sodium sulfate and concentrated under reduced pressure. The residue was purified by preparative HPLC to afford 40 mg (40% yield) of the title compound:

¹H NMR (400 MHz, CDCl₃) δ ppm 8.65 (dd, 1H) 7.83 (dd, 1H) 7.50-7.59 (m, 3H) 7.34-7.42 (m, 2H) 6.91 (d, 1H) 6.63 (d, 1H) 6.23-6.62 (m, 1H) 5.36 (s, 2H) 2.25 (s, 3H) 2.01-2.12 (m, 1H) 0.91-0.98 (m, 2H) 0.47-0.61 (m, 2H); MS (ES+) m/z: 431 [M+1]⁺.

### Example 33

### 5-(3-Cyclopropyl-4-methoxy-phenyl)-5-(3-pyrimidin-5-yl-phenyl)-5H-pyrrolo[3,4-b]pyridin-7-ylamine

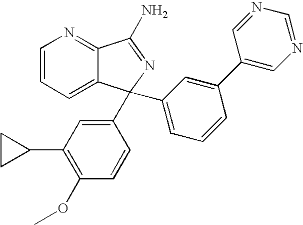

Pyrimidin-5-ylboronic acid (60 mg, 0.42 mmol), 7-(3-bromo-phenyl)-7-(3-cyclopropyl-4-methoxy-phenyl)-7H-pyrrolo[3,4-b]pyridin-5-ylamine (140 mg, 0.32 mmol), Pd(dppf)Cl₂-dichloromethane complex (24 mg, 0.03 mmol) and cesium carbonate (315 mg, 0.97 mmol) were dissolved in a mixture of DME (3.0 mL), EtOH (1.5 mL) and water (0.5 mL). The reaction mixture was degassed and purged with nitrogen for 10 minutes and then heated in a microwave reactor at 120° C. for 30 minutes. The mixture was diluted with ethyl acetate, filtered and concentrated under reduced pressure. The residue was purified using preparative HPLC to obtain 47 mg (34% yield) of the title compound:

¹H NMR (400 MHz, CDCl₃) δ ppm 9.16 (s, 1H) 8.85 (s, 2H) 8.61 (d, 1H) 7.87 (d, 1H) 7.50 (s, 1H) 7.31-7.47 (m, 4H) 7.08 (dd, 1H) 6.84 (d, 1H) 6.74 (d, 1H) 5.30 (s, 2H) 3.83 (s, 3H) 2.04-2.14 (m, 1H) 0.84 (dd, 2H) 0.47-0.60 (m, 2H); MS (ES+) m/z: 433.94 [M+1]⁺.

### Example 34

### 5-[4-Difluoromethoxy-3-(2-fluoro-ethyl)-phenyl]-5-(3-pyrimidin-5-yl-phenyl)-5H-pyrrolo[3,4-b]pyridin-7-ylamine

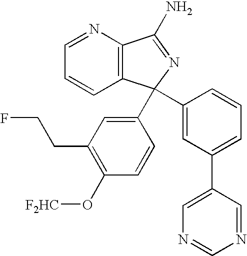

A mixture of 5-(3-bromo-phenyl)-5-[4-difluoromethoxy-3-(2-fluoro-ethyl)-phenyl]-5H-pyrrolo[3,4-b]pyridin-7-ylamine (494 mg, 1.04 mmol), pyrimidin-5-ylboronic acid (192.8 mg, 1.56 mmol) and potassium carbonate (430.3 mg, 3.11 mmol) in a mixture of DME, water and ethanol (6:2:1, 15 mL) was degassed using nitrogen for 10 minutes. Pd(dppf)Cl₂ (75.9 mg, 0.10 mmol) was added in one portion and the reaction mixture was heated at 100° C. in a sealed tube for 1.5 hours. The mixture was cooled to room temperature, diluted with ethyl acetate (50 mL) and filtered. The filtrate was washed with water, brine, dried over sodium sulfate and concentrated in vacuo. The residue was purified by preparative HPLC to afford 270 mg (55% yield) of the title compound:

¹H NMR (400 MHz, CDCl₃) δ ppm 9.17 (s, 1H) 8.85 (s, 2H) 8.66 (d, 1H) 7.92 (d, 1H) 7.18-7.59 (m, 7H) 7.03 (d, 1H) 6.50 (t, 1H) 5.40 (br.s., 2H) 4.62 (t, 1H) 4.50 (t, 1H) 3.04 (m, 1H) 2.97 (m, 1H); ¹⁹F NMR (376 MHz, CHLOROFORM-d) δ ppm-82.17, -219.24; MS (ES+) m/z: 476.01 [M+1]⁺.

### Example 35

### 5-(5-Methoxy-4,6-dimethyl-pyridin-2-yl)-5-(3-pyrimidin-5-yl-phenyl)-5H-pyrrolo[3,4-b]pyridin-7-ylamine

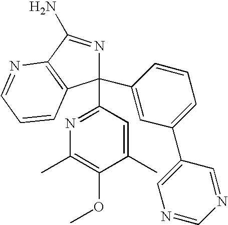

A mixture of 5-(3-bromo-phenyl)-5-(5-methoxy-4,6-dimethyl-pyridin-2-yl)-5H-pyrrolo[3,4-b]pyridin-7-ylamine (0.15 g, 0.35 mmol), pyrimidine-5-boronic acid (0.066 g, 0.53 mmol), potassium carbonate (0.15 g, 1.06 mmol), and Pd(dppf)Cl₂ (0.03 g, 0.044 mmol) in a mixture of DME, water and ethanol (6:3:1, 5 mL) was degassed with nitrogen for 30 minutes. The reaction mixture was heated in a sealed tube at 100° C. for 1 hour. The mixture was diluted with ethyl acetate and filtered. The filtrate was concentrated under reduced pressure. The residue was purified by preparative HPLC to afford 35 mg (25% yield) of the title compound:

¹H NMR (400 MHz, CDCl₃) δ ppm 9.16 (s, 1H), 8.85 (s, 2H), 8.58 (d, 1H), 8.49 (d, 1H), 7.63 (s, 1H), 7.53-7.50 (m, 2H), 7.40-7.34 (m, 3H), 5.41 (br, s, NH₂), 3.70 (s, 3H), 2.47 (s, 3H), 2.25 (s, 3H); MS (ES+) m/z: 422.86 [M+1]⁺.

### Example 36

### 5-(3-Fluoro-4-methoxy-5-methylphenyl)-5-(3-(pyrimidin-5-yl)phenyl)-5H-pyrrolo[3,4-b]pyridin-7-amine

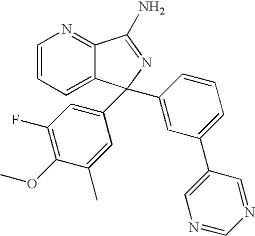

5-(3-Bromophenyl)-5-(3-fluoro-4-methoxy-5-methylphenyl)-5H-pyrrolo[3,4-b]pyridin-7-amine (125 mg, 0.29 mmol), pyrimidine-5-boronic acid (40.0 mg, 0.32 mmol) and [1,1′-bis(diphenylphosphino)ferrocene]palladium(II) chloride (12.06 mg, 0.01 mmol) were dissolved in DMF (1.5 mL). Aqueous potassium carbonate (0.440 mL, 0.88 mmol) was added and the mixture was microwaved at 150° C. for 15 min. Methanol (2 mL) was added and the mixture was filtered and purified by preparative HPLC to give 5-(3-Fluoro-4-methoxy-5-methylphenyl)-5-(3-(pyrimidin-5-yl)phenyl)-5H-pyrrolo[3,4-b]pyridin-7-amine (36 mg, 28% yield):

¹H NMR (400 MHz, CDCl₃) δ ppm 9.19 (s, 1H), 8.89 (s, 2H), 8.67 (dd, 1H), 7.93 (dd, 1 H), 7.55 (dt, 1H), 7.39-7.51 (m, 4H), 6.84-6.89 (m, 2H), 5.77 (br s., 2H), 3.88 (d, 3H), 2.20 (s, 3H); MS (ES+) m/z 426 [M+1]⁺.

### Example 37

### 5-(7-Amino-5-(2-(trifluoromethyl)pyridin-4-yl)-5H-pyrrolo[3,4-b]pyridin-5-yl)-2′-fluoro-5′-methoxybiphenyl-2-ol

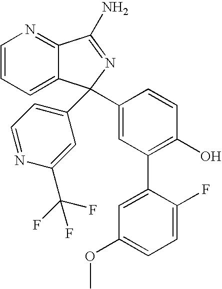

The title compound was synthesized as described for Example 18 in 28% yield, starting from 4-(7-amino-5-(2-(trifluoromethyl)pyridin-4-yl)-5H-pyrrolo[3,4-b]pyridin-5-yl)-2-bromophenol (140 mg, 0.31 mmol), and 2-fluoro-5-methoxyphenylboronic acid (63.6 mg, 0.37 mmol):

¹H NMR (500 MHz, DMSO-d₆) δ ppm 8.59-8.76 (m, 2H) 8.40 (dd, 1H) 7.76 (s, 1H) 7.70 (dd, 1H) 7.51 (dd, 1H) 7.20 (dd, 1H) 7.06-7.16 (m, 2H) 6.83-7.07 (m, 4H) 6.80 (dd, 1H) 3.72 (s, 3H); MS (ES) m/z 495 [M+1]⁺.

### Example 38

### 5-(7-Amino-5-(2-(trifluoromethyl)pyridin-4-yl)-5H-pyrrolo[3,4-b]pyridin-5-yl)-2′-fluorobiphenyl-2-ol

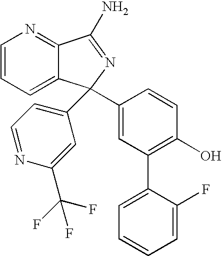

The title compound was synthesized as described for Example 18 in 17% yield, starting from 4-(7-amino-5-(2-(trifluoromethyl)pyridin-4-yl)-5H-pyrrolo[3,4-b]pyridin-5-yl)-2-bromophenol (271 mg, 0.60 mmol) and 2-fluorobenzeneboronic acid (101 mg, 0.72 mmol):

¹H NMR (600 MHz, DMSO-d₆) δ ppm 9.68 (br. s., 1H), 8.67 (t, 2H), 8.34-8.45 (m, 1H) 7.76 (s, 1H) 7.70 (d, 1H) 7.47-7.55 (m, 1H) 7.31-7.40 (m, 1H) 7.24-7.32 (m, 1H) 7.20-7.21 (m, 0H) 7.15-7.25 (m, 3H) 7.10-7.15 (m, 1H) 6.97 (br. s., 2H) 6.88 (d, 1H); MS (ES) m/z 466 [M+1]⁺.

### Assays

The level of activity of the compounds was tested using the following methods:

### TR-FRET Assay

The β-secretase enzyme used in the TR-FRET is prepared as follows:

The cDNA for the soluble part of the human β-Secretase (AA 1-AA 460) was cloned using the ASP2-Fc10-1-IRES-GFP-neoK mammalian expression vector. The gene was fused to the Fc domain of IgG1 (affinity tag) and stably cloned into HEK 293 cells. Purified sBACE-Fc was stored in ±80° C. in Tris buffer, pH 9.2 and had a purity of 95%.

The enzyme (truncated form) was diluted to 6 μg/mL (stock 1.3 mg/mL) and the substrate (Europium)CEVNLDAEFK(Qsy7) to 200 nM (stock 120 μM) in reaction buffer (NaAcetate, chaps, triton x-100, EDTA pH4.5). The robotic systems Biomek FX and Velocity 11 were used for all liquid handling and the enzyme and substrate solutions were kept on ice until they were placed in the robotic system. Enzyme (9 μl) was added to the plate then 1 μl of compound in dimethylsulphoxide was added, mixed and pre-incubated for 10 minutes. Substrate (10 μl) was then added, mixed and the reaction proceeded for 15 minutes at room temperature. The reaction was stopped with the addition of Stop solution (7 μl, NaAcetate, pH 9). The fluorescence of the product was measured on a Victor II plate reader with an excitation wavelength of 340 nm and an emission wavelength of 615 nm. The assay was performed in a Costar 384 well round bottom, low volume, non-binding surface plate (Corning #3676). The final concentration of the enzyme was 2.7 μg/ml; the final concentration of substrate was 100 nM (Km of ˜250 nM). The dimethylsulphoxide control, instead of test compound, defined the 100% activity level and 0% activity was defined by wells lacking enzyme (replaced with reaction buffer). A control inhibitor was also used in dose response assays and had an IC₅₀ of ±575 nM.

sAPPβ Release Assay

SH-SY5Y cells were cultured in DMEM/F-12 with Glutamax, 10% FCS and 1% non-essential aminoacids and cryopreserved and stored at −140° C. at a concentration of 7.5×106 cells per vial. Thaw cells and seed at a conc. of 1.5×105/ml in DMEM/F-12 with Glutamax, 10% FCS and 1% non-essential aminoacids to a 96-well tissue culture treated plate, 100 μl cell susp/well. The cell plates were then incubated for 7 hours at 37° C., 5% CO2. The cell medium was removed, followed by addition of 90 μl compound diluted in DMEM/F-12 with Glutamax, 10% FCS, 1% non-essential aminoacids and 1% PeSt to a final conc. of 1% DMSO. The compounds were incubated with the cells for 16 h (over night) at 37° C., 5% CO₂. Meso Scale Discovery (MSD) plates were used for the detection of sAPPβ release. MSD sAPPβ plates were blocked in 3% BSA in Tris wash buffer (150 μl/well) for 1 hour in RT and washed 4 times in Tris wash buffer (150 μl/well). 50 μl of medium was transferred to the pre-blocked and washed MSD sAPPβ microplates, and the cell plates were further used in an ATP assay to measure cytotoxicity. The MSD plates were incubated with shaking in RT for 1 hour followed by washing 4 times. 25 μl detection antibody was added (1 nM) per well followed by incubation with shaking in RT for 1 h and washing 4 times. 150 μl Read Buffer was added per well and the plates were read in a SECTOR Imager.

### ATP Assay

As indicated in the sAPPβ release assay, after transferring 50 μL medium from the cell plates for sAPPβ detection, the plates were used to analyse cytotoxicity using the ViaLight™ Plus cell proliferation/cytotoxicity kit from Cambrex BioScience that measures total cellular ATP. The assay was performed according to the manufacture's protocol. Briefly, 25 μL cell lysis reagent was added per well. The plates were incubated at room temperature for 10 min. Two min after addition of 50 μL reconstituted ViaLight™Plus ATP reagent, the luminescence was measured in a Wallac Victor2 1420 multilabel counter.

### Results

Typical IC₅₀ values for the compounds of the present invention are in the range of about 0.1 to about 30,000 nM. Biological data on exemplified final compounds is given below in Table I.

TABLE I

| Example No. | IC50 in TR-FRET assay |
|---|---|
| 1 | 400 |
| 2 | 350 |
| 3 | 560 |
| 4 | 1800 |
| 5 | 2400 |
| 6 | 156 |
| 7 | 248 |
| 8 | 610 |
| 9 | 33 |
| 10 | 639 |
| 11 | 2120 |
| 12 | 47 |
| 13 | 38 |
| 14 | 593 |
| 15 | 96 |
| 16 | 1600 |
| 17 | 1140 |
| 18 | 204 |
| 19 | 359 |
| 20 | 445 |
| 21 | 239 |
| 22 | 1800 |
| 23 | 288 |
| 24 | 212 |
| 25, Isomer 1 | >31600 |
| 25, Isomer 2 | 176 |
| 26 | 42 |
| 27 | 90 |
| 28 | 1560 |
| 29 | 106 |
| 30 | 119 |
| 31 | 815 |
| 32 | 61 |
| 33 | 222 |
| 34 | 30 |
| 35 | 125 |
| 36 | 41 |
| 37 | 206 |
| 38 | 658 |

1. A compound according to formula (I) or a pharmaceutically acceptable salt thereof, wherein:
formula (I) corresponds to: 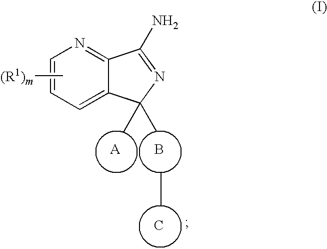
R¹ is selected from halogen, cyano, NO₂, SO₂R², C₁₋₆alkyl, C₂₋₆alkenyl, C₂₋₆alkynyl, NR³R⁴, OR², C(O)R², C(O)NR³R⁴, and COOR², wherein:
the C₁₋₆alkyl, C₂₋₆alkenyl, or C₂₋₆alkynyl is optionally substituted with one or more R⁷;
R² is selected from C₁₋₆alkyl, C₂₋₆alkenyl, and C₂₋₆alkynyl, wherein:
the C₁₋₆alkyl, C₂₋₆alkenyl, or C₂₋₆alkynyl is optionally substituted with one or more R⁷;
as to R³ and R⁴:
R³ and R⁴ are independently selected from hydrogen, C₁₋₆alkyl, C₂₋₆alkenyl, C₂₋₆alkynyl, aryl, heteroaryl, heterocyclyl, and carbocyclyl, wherein:
the C₁₋₆alkyl, C₂₋₆alkenyl, C₂₋₆alkynyl, aryl, heteroaryl, heterocyclyl or carbocyclyl is optionally substituted with one or more R⁷; or
R³ and R⁴, together with the atom to which they are attached, form a 4 to 7 membered ring;
Substituent A is selected from aryl and heteroaryl, wherein:
the aryl or heteroaryl is optionally substituted with one or more R⁵;
Substituent B is selected from aryl and heteroaryl, wherein:
the aryl or heteroaryl is optionally substituted with one or more R⁶;
Substituent C is selected from hydrogen, halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein:
the aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, or C₂₋₆alkenylC₃₋₆cycloalkyl is optionally substituted with one to three R⁷;
R⁵ is selected from halo, cyano, C₁₋₆alkyl, C₁₋₆haloalkyl, C₃₋₆cycloalkyl, OC₁₋₆alkyl, OC₂₋₆alkenyl, and OC₁₋₆alkylaryl, wherein:
the C₁₋₆alkyl, C₁₋₆haloalkyl, C₃₋₆cycloalkyl, OC₁₋₆alkyl, OC₂₋₆alkenyl, or OC₁₋₆alkylaryl is optionally substituted with one to three R⁷;
R⁶ is selected from halogen, hydroxy, and cyano;
R⁷ is selected from halogen, cyano, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, C(O)OC₁₋₃alkyl, and NR⁸R⁹, wherein:
the C₁₋₆alkyl, SO₂C₁₋₃alkyl, OC₁₋₃alkyl, OC₁₋₃haloalkyl, C₁₋₃alkylOH, C₁₋₃alkylNR⁸R⁹, or C(O)OC₁₋₃alkyl is optionally substituted with one or more R¹⁰;
as to R⁸ and R⁹:
R⁸ and R⁹ are independently selected from hydrogen, C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl, and carbocyclyl, wherein:
the C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl, or carbocyclyl is optionally substituted with one or more R¹⁰; or
R⁸ and R⁹, together with the atom to which they are attached, form a 4 to 6 membered ring;
R¹⁰ is selected from halo, C₁₋₃alkyl, OC₁₋₃alkyl, and OC₁₋₃haloalkyl;
R¹¹ and R¹² are independently selected from hydrogen, C₁₋₃alkyl, and C₁₋₃haloalkyl;
m is selected from 0, 1, and 2.

2. A compound according to formula (I) or a pharmaceutically acceptable salt thereof, wherein:
formula (I) corresponds to: 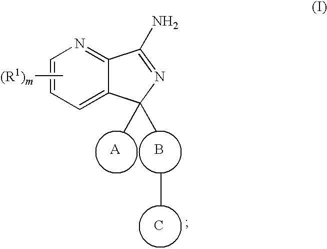
R¹ is selected from halogen, cyano, NO₂, SO₂R², C₁₋₆alkyl, C₂₋₆alkenyl, C₂₋₆alkynyl, NR³R⁴, OR², C(O)R², C(O)NR³R⁴, and COOR², wherein:
the C₁₋₆alkyl, C₂₋₆alkenyl, or C₂₋₆alkynyl is optionally substituted with one or more R⁷;
R² is selected from C₁₋₆alkyl, C₂₋₆alkenyl, and C₂₋₆alkynyl, wherein:
the C₁₋₆alkyl, C₂₋₆alkenyl, or C₂₋₆alkynyl is optionally substituted with one or more R⁷;
as to R³ and R⁴:
R³ and R⁴ are independently selected from hydrogen, C₁₋₆alkyl, C₂₋₆alkenyl, C₂₋₆alkynyl, aryl, heteroaryl, heterocyclyl, and carbocyclyl, wherein:
the C₁₋₆alkyl, C₂₋₆alkenyl, C₂₋₆alkynyl, aryl, heteroaryl, heterocyclyl, or carbocyclyl is optionally substituted with one or more R⁷; or
R³ and R⁴, together with the atom to which they are attached, form a 4 to 7 membered ring;
Substituent A is selected from aryl and heteroaryl, wherein:
the aryl or heteroaryl is optionally substituted with one or more R⁵;
Substituent B is selected from aryl and heteroaryl, wherein:
the aryl or heteroaryl is optionally substituted with one or more R⁶;
Substituent C is selected from halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein:
the aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, or C₂₋₆alkenylC₃₋₆cycloalkyl is optionally substituted with one to three R⁷;
R⁵ is selected from halo, cyano, C₁₋₆alkyl, C₁₋₆haloalkyl, C₃₋₆cycloalkyl, OC₁₋₆alkyl, OC₂₋₆alkenyl, and OC₁₋₆alkylaryl, wherein:
the C₁₋₆alkyl, C₁₋₆haloalkyl, C₃₋₆cycloalkyl, OC₁₋₆alkyl, OC₂₋₆alkenyl, or OC₁₋₆alkylaryl is optionally substituted with one to three R⁷;
R⁶ is selected from halogen, hydroxy, and cyano;
R⁷ is selected from halogen, cyano, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, C(O)OC₁₋₃alkyl, and NR⁸R⁹, wherein:
the C₁₋₆alkyl, SO₂C₁₋₃alkyl, OC₁₋₃alkyl, OC₁₋₃haloalkyl, C₁₋₃alkylOH, C₁₋₃alkylNR⁸R⁹, or C(O)OC₁₋₃alkyl is optionally substituted with one or more R¹⁰;
as to R⁸ and R⁹:
R⁸ and R⁹ are independently selected from hydrogen, C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl, and carbocyclyl, wherein:
the C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl, or carbocyclyl is optionally substituted with one or more R¹⁰; or
R⁸ and R⁹, together with the atom to which they are attached, form a 4 to 6 membered ring;
R¹⁰ is selected from halo, C₁₋₃alkyl, OC₁₋₃alkyl, and OC₁₋₃haloalkyl;
R¹¹ and R¹² are independently selected from hydrogen, C₁₋₃alkyl, and C₁₋₃haloalkyl; and
m is selected from 0, 1, and 2.

3. A compound or pharmaceutically acceptable salt thereof according to claim 1, wherein:
R¹ is selected from halogen, cyano, NO₂, SO₂R², C₁₋₆alkyl, NR³R⁴, OR², C(O)R², C(O)NR³R⁴, and COOR², wherein:
the C₁₋₆alkyl is optionally substituted with one or more R⁷;
as to R³ and R⁴:
R³ and R⁴ are independently selected from hydrogen, C₁₋₆alkyl, aryl, heteroaryl, heterocyclyl, and carbocyclyl, wherein:
the C₁₋₆alkyl, aryl, heteroaryl, heterocyclyl, or carbocyclyl is optionally substituted with one or more R⁷; or
R³ and R⁴, together with the atom to which they are attached, form a 4 to 7 membered ring;
Substituent C is selected from halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein:
the aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, or C₂₋₆alkenylC₃₋₆cycloalkyl is optionally substituted with one to three R⁷;
R⁷ is selected from halogen, cyano, C₁₋₆ alkyl, SO₂C₁₋₃ alkyl, OC₁₋₃ alkyl, OC₁₋₃ haloalkyl, C₁₋₃ alkylOH, C₁₋₃ alkylNR⁸R⁹, cyano, and C(O)OC₁₋₃ alkyl, wherein:
the C₁₋₆alkyl, SO₂C₁₋₃alkyl, OC₁₋₃alkyl, OC₁₋₃haloalkyl, C₁₋₃alkylOH, C₁₋₃alkylNR⁸R⁹, or C(O)OC₁₋₃alkyl is optionally substituted with one or more R¹⁰; and
as to R⁸ and R⁹:
R⁸ and R⁹ are independently selected from hydrogen, C₁₋₆alkyl, C₁₋₆haloalkyl, C₁₋₃alkylNR¹¹R¹², C₁₋₃ alkylOaryl, heteroaryl, heterocyclyl, and carbocyclyl, wherein:
the C₁₋₆alkyl, C₁₋₆haloalkyl, C₁₋₃alkylNR¹¹R¹², C₁₋₃alkylOaryl, heteroaryl, heterocyclyl, or carbocyclyl is optionally substituted with one or more R¹⁰; or
R⁸ and R⁹, together with the atom to which they are attached, form a 4 to 6 membered ring.

4. A compound or pharmaceutically acceptable salt thereof according to claim 1, wherein:
R¹ is selected from halogen, cyano, NO₂, SO₂R², C₁₋₆alkyl, NR³R⁴, OR², and C(O)R², wherein:
the C₁₋₆alkyl is optionally substituted with one or more R⁷;
R²C₁₋₆alkyl optionally substituted with one or more R⁷;
R³ and R⁴ are independently selected from hydrogen, C₁₋₆alkyl, aryl, heteroaryl, heterocyclyl, and carbocyclyl, wherein:
the C₁₋₆alkyl, aryl, heteroaryl, heterocyclyl, or carbocyclyl is optionally substituted with one or more R⁷;
Substituent C is selected from halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein:
the aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, or C₂₋₆alkenylC₃₋₆cycloalkyl is optionally substituted with one to three R⁷;
R⁵ is selected from halo, cyano, C₁₋₆alkyl, C₁₋₆haloalkyl, C₃₋₆cycloalkyl, OC₁₋₆alkyl, OC₂₋₆alkenyl, and OC₁₋₆alkylaryl, wherein:
the C₁₋₆alkyl, C₁₋₆haloalkyl, C₃₋₆cycloalkyl, OC₁₋₆alkyl, OC₂₋₆alkenyl, or OC₁₋₆alkylaryl is optionally substituted with one to three R⁷;
R⁶ is selected from halogen and hydroxy;
R⁷ is selected from halogen, cyano, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, cyano, and C(O)OC₁₋₃alkyl, wherein:
the C₁₋₆alkyl, SO₂C₁₋₃alkyl, OC₁₋₃alkyl, OC₁₋₃haloalkyl, C₁₋₃alkylOH, C₁₋₃alkylNR⁸R⁹, or C(O)OC₁₋₃alkyl is optionally substituted with one or more R¹⁰;
R⁸ and R⁹ are independently selected from hydrogen, C₁₋₆alkyl, C₁₋₆haloalkyl, C₁₋₃alkylNR¹¹R¹², C₁₋₃alkylOaryl, heteroaryl, heterocyclyl, and carbocyclyl, wherein:
the C₁₋₆alkyl, C₁₋₆haloalkyl, C₁₋₃alkylNR¹¹R¹², C₁₋₃alkylOaryl, heteroaryl, heterocyclyl, or carbocyclyl is optionally substituted with one or more R¹⁰;
R¹⁰ is selected from halo, C₁₋₃alkyl, OC₁₋₃alkyl, and OC₁₋₃haloalkyl;
R¹¹ and R¹² are independently selected from hydrogen, C₁₋₃alkyl, and C₁₋₃haloalkyl; and
m is selected from 0 and 1.

5. A compound or pharmaceutically acceptable salt thereof according to claim 1, wherein Substituent A is heteroaryl.

6. A compound or pharmaceutically acceptable salt thereof according to claim 5, wherein Substituent A is selected from pyridinyl and pyrimidinyl.

7. A compound or pharmaceutically acceptable salt thereof according to claim 1, wherein Substituent A is aryl.

8. A compound or pharmaceutically acceptable salt thereof according to claim 7, wherein Substituent A is phenyl.

9. A compound or pharmaceutically acceptable salt thereof according to claim 1, wherein Substituent A is selected from aryl and heteroaryl.

10. A compound or pharmaceutically acceptable salt thereof according to claim 1, wherein Substituent A is selected from aryl and heteroaryl, wherein:
the aryl or heteroaryl is substituted with one or more R⁵.

11. A compound or pharmaceutically acceptable salt thereof according to claim 1, wherein Substituent C is selected from halogen, cyano, aryl, heteroaryl, heterocyclyl, C₃₋₆cycloalkyl, C₁₋₆alkyl, C₁₋₆alkylC₃₋₆heterocyclyl, C₁₋₆alkylaryl, and C₁₋₆alkylheteroaryl.

12. A compound or pharmaceutically acceptable salt thereof according to claim 1, wherein Substituent C is selected from halogen, cyano, aryl, heteroaryl, and C₁₋₆alkyl.

13. A compound or pharmaceutically acceptable salt thereof according to claim 1, wherein Substituent C is selected from hydrogen, halogen, cyano, aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl.

14. A compound or pharmaceutically acceptable salt thereof according to claim 1, wherein Substituent C is selected from aryl, heteroaryl, heterocyclyl, C₃₋₆cycloalkyl, C₃₋₆cycloalkenyl, C₁₋₆alkyl, C₂₋₆alkenyl, C₂₋₆alkynyl, C₁₋₆alkylC₃₋₆cycloalkyl, C₁₋₆alkylC₃₋₆heterocyclyl, C₁₋₆alkylaryl, C₁₋₆alkylheteroaryl, and C₂₋₆alkenylC₃₋₆cycloalkyl, wherein:
any such group is substituted with one to three R⁷.

15. A compound or pharmaceutically acceptable salt thereof according to claim 14, wherein R⁷ is selected from halogen, cyano, C₁₋₆alkyl, SO₂C₁₋₃alkyl, OC₁₋₃alkyl, and OC₁₋₃haloalkyl.

16. A compound or pharmaceutically acceptable salt thereof according to claim 1, wherein R⁶ is selected from fluoro, chloro, and hydroxy.

17. A compound or pharmaceutically acceptable salt thereof according to claim 1, wherein m is 0.

18. A compound or pharmaceutically acceptable salt thereof according to claim 1, wherein:
Substituent C is selected from halogen, cyano, aryl, heteroaryl, and C₁₋₆alkyl, wherein:
the aryl, heteroaryl, or C₁₋₆alkyl is optionally substituted with one to three R⁷;
R⁵ is selected from halo, cyano, C₁₋₆alkyl, C₁₋₆haloalkyl, C₃₋₆cycloalkyl, OC₂₋₆alkenyl, and OC₁₋₆alkylaryl, wherein:
the C₁₋₆alkyl, C₁₋₆haloalkyl, C₃₋₆cycloalkyl, OC₂₋₆alkenyl, or OC₁₋₆alkylaryl is optionally substituted with one to three R⁷;
R⁶ is selected from halogen and hydroxy;
R⁷ is selected from halogen, cyano, C₁₋₆alkyl, SO₂C₁₋₃alkyl, OC₁₋₃alkyl, OC₁₋₃haloalkyl, wherein:
the C₁₋₆alkyl, SO₂C₁₋₃alkyl, OC₁₋₃alkyl, or OC₁₋₃haloalkyl is optionally substituted with one or more R¹⁰;
R¹⁰ is halo; and
m is selected from 0 and 1.

19. A compound or pharmaceutically acceptable salt thereof according to claim 1, wherein:
Substituent A is heteroaryl, wherein:
the heteroaryl is optionally substituted with one or more R⁵;
Substituent B is aryl;
Substituent C is selected from aryl and heteroaryl, wherein:
the aryl or heteroaryl is optionally substituted with one to three R⁷;
R⁵ is selected from C₁₋₆alkyl, OC₂₋₆alkenyl, and C₁₋₆haloalkyl, wherein:
the C₁₋₆alkyl or OC₂₋₆alkenyl is optionally substituted with one to three R⁷;
R⁷ is selected from halogen and cyano; and
m is 1.

20. A compound or pharmaceutically acceptable salt thereof according to claim 18, wherein Substituent B is phenyl.

21. A compound or pharmaceutically acceptable salt thereof according to claim 1, wherein R⁵ is selected from halo, cyano, C₁₋₆alkyl, C₁₋₆haloalkyl, C₃₋₆cycloalkyl, OC₁₋₆alkyl, and OC₁₋₆alkylaryl, wherein:
the C₁₋₆alkyl, C₁₋₆haloalkyl, C₃₋₆cycloalkyl, OC₁₋₆alkyl, or OC₁₋₆alkylaryl is optionally substituted with one to three R⁷;

22. A compound or pharmaceutically acceptable salt thereof according to claim 1, wherein R⁶ is selected from halogen and cyano.

23. A compound or pharmaceutically acceptable salt thereof, wherein the compound is selected from:
5-(3′-chlorobiphenyl-3-yl)-5-(2-(trifluoromethyl)pyridin-4-yl)-5H-pyrrolo[3,4-b]pyridin-7-amine;
5-(3-(pyridin-3-yl)phenyl)-5-(2-(trifluoromethyl)pyridin-4-yl)-5H-pyrrolo[3,4-b]pyridin-7-amine;
5-(3-(pyridin-3-yl)phenyl)-5-(2-(trifluoromethyl)pyridin-4-yl)-5H-pyrrolo[3,4-b]pyridin-7-amine;
5-(2,6-dimethylpyridin-4-yl)-5-(3-(pyrimidin-5-yl)phenyl)-5H-pyrrolo[3,4-b]pyridin-7-amine;
5-(3-(7-amino-5-(2,6-dimethylpyridin-4-yl)-5H-pyrrolo[3,4-b]pyridin-5-yl)phenyl)nicotinonitrile;
5-(3,5-difluoro-4-methoxyphenyl)-5-(4-fluoro-3-(pyrimidin-5-yl)phenyl)-5H-pyrrolo[3,4-b]pyridin-7-amine;
5-(3-chloro-4-methoxyphenyl)-5-(3-(pyrimidin-5-yl)phenyl)-5H-pyrrolo[3,4-b]pyridin-7-amine;
5-(3-chloro-4-methoxyphenyl)-5-(3-(pyrazin-2-yl)phenyl)-5H-pyrrolo[3,4-b]pyridin-7-amine;
5-(3-cyclopropyl-4-(difluoromethoxy)phenyl)-5-(3-(pyrimidin-5-yl)phenyl)-5H-pyrrolo[3,4-b]pyridin-7-amine;
3-chloro-5-(2-methylpyridin-4-yl)-5-(3-(pyrimidin-5-yl)phenyl)-5H-pyrrolo[3,4-b]pyridin-7-amine;
5-(4-methoxyphenyl)-3-methyl-5-(3-(pyrimidin-5-yl)phenyl)-5H-pyrrolo[3,4-b]pyridin-7-amine;
5-(4-(difluoromethoxy)-3,5-dimethylphenyl)-5-(3-(pyrimidin-5-yl)phenyl)-5H-pyrrolo[3,4-b]pyridin-7-amine;
5-(4-(difluoromethoxy)-3,5-dimethylphenyl)-5-(3-(pyrazin-2-yl)phenyl)-5H-pyrrolo[3,4-b]pyridin-7-amine;
5-(4-fluoro-3,5-dimethylphenyl)-5-(3-(pyrimidin-5-yl)phenyl)-5H-pyrrolo[3,4-b]pyridin-7-amine;
5-(3-fluoro-4-methoxy-5-methylphenyl)-5-(3-(pyrazin-2-yl)phenyl)-5H-pyrrolo[3,4-b]pyridin-7-amine;
5-(3-(pyrimidin-5-yl)phenyl)-5-(2-(2,2,2-trifluoroethoxy)pyridin-4-yl)-5H-pyrrolo[3,4-b]pyridin-7-amine;
5-(2-(2,2-difluorovinyloxy)pyridin-4-yl)-5-(3-(pyrimidin-5-yl)phenyl)-5H-pyrrolo[3,4-b]pyridin-7-amine;
5-(4-(difluoromethoxy)-3-fluorophenyl)-5-(3-(pyrimidin-5-yl)phenyl)-5H-pyrrolo[3,4-b]pyridin-7-amine;
5-(3-(4-methoxypyridin-2-yl)phenyl)-5-(2-(trifluoromethyl)pyridin-4-yl)-5H-pyrrolo[3,4-b]pyridin-7-amine;
5-(2-(difluoromethyl)-6-methylpyridin-4-yl)-5-(3-(pyrimidin-5-yl)phenyl)-5H-pyrrolo[3,4-b]pyridin-7-amine;
5-(3-(5-chloropyridin-3-yl)phenyl)-5-(2-(trifluoromethyl)pyridin-4-yl)-5H-pyrrolo[3,4-b]pyridin-7-amine;
2-(3-(7-dmino-5-(2-(trifluoromethyl)pyridin-4-yl)-5H-pyrrolo[3,4-b]pyridin-5-yl)phenyl)isonicotinonitrile;
5-(3-(difluoromethyl)-4-methoxyphenyl)-5-(3-(pyrazin-2-yl)phenyl)-5H-pyrrolo[3,4-b]pyridin-7-amine;
5-(3-(difluoromethyl)-4-methoxyphenyl)-5-(3-(pyrimidin-5-yl)phenyl)-5H-pyrrolo[3,4-b]pyridin-7-amine;
5-(4-(fluoromethoxy)-3,5-dimethylphenyl)-5-(3-(pyrimidin-5-yl)phenyl)-5H-pyrrolo[3,4-b]pyridin-7-amine;
5-(4-(fluoromethoxy)-3,5-dimethylphenyl)-5-(3-(pyrazin-2-yl)phenyl)-5H-pyrrolo[3,4-b]pyridin-7-amine;
5-(2-(3-fluoropropoxy)pyridin-4-yl)-5-(3-(pyrimidin-5-yl)phenyl)-5H-pyrrolo[3,4-b]pyridin-7-amine;
5-(4-difluoromethoxy-3,5-dimethyl-phenyl)-5-(2-pyrimidin-5-yl-pyridin-4-yl)-5H-pyrrolo[3,4-b]pyridin-7-ylamine;
5-(3-cyclopropyl-4-difluoromethoxy-5-methyl-phenyl)-5-(2-pyrimidin-5-yl-pyridin-4-yl)-5H-pyrrolo[3,4-b]pyridin-7-ylamine;
5-[3-cyclopropyl-4-(difluoromethoxy)-5-methyl-phenyl]-5-phenyl-pyrrolo[3,4-b]pyridin-7-amine;
3-[7-amino-5-(3-cyclopropyl-4-difluoromethoxy-5-methyl-phenyl)-5H-pyrrolo[3,4-b]pyridin-5-yl]-benzonitrile;
5-(3-cyclopropyl-4-methoxy-phenyl)-5-(3-pyrimidin-5-yl-phenyl)-5H-pyrrolo[3,4-b]pyridin-7-ylamine;
5-[4-difluoromethoxy-3-(2-fluoro-ethyl)-phenyl]-5-(3-pyrimidin-5-yl-phenyl)-5H-pyrrolo[3,4-b]pyridin-7-ylamine;
5-(5-methoxy-4,6-dimethyl-pyridin-2-yl)-5-(3-pyrimidin-5-yl-phenyl)-5H-pyrrolo[3,4-b]pyridin-7-ylamine;
5-(3-fluoro-4-methoxy-5-methylphenyl)-5-(3-(pyrimidin-5-yl)phenyl)-5H-pyrrolo[3,4-b]pyridin-7-amine;
5-(7-amino-5-(2-(trifluoromethyl)pyridin-4-yl)-5H-pyrrolo[3,4-b]pyridin-5-yl)-2′-fluoro-5′-methoxybiphenyl-2-ol; and
5-(7-amino-5-(2-(trifluoromethyl)pyridin-4-yl)-5H-pyrrolo[3,4-b]pyridin-5-yl)-2′-fluorobiphenyl-2-ol.

24. A pharmaceutical composition, wherein the composition comprises:
a therapeutically effective amount of a compound or pharmaceutically acceptable salt thereof according to claim 1; and
a pharmaceutically acceptable excipient, carrier, or diluent.

25-31. (canceled)

32. A method of inhibiting activity of BACE, wherein the method comprises contacting BACE with a compound or pharmaceutically acceptable salt thereof according to claim 1.

33. A method of treating or preventing an Aβ-related pathology in a mammal, comprising administering to the mammal a therapeutically effective amount of a compound or pharmaceutically acceptable salt thereof according to claim 1.

34. The method of claim 33, wherein the Aβ-related pathology is selected from Downs syndrome, a β-amyloid angiopathy, cerebral amyloid angiopathy, hereditary cerebral hemorrhage, a disorder associated with cognitive impairment, mild cognitive impairment, Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy, and cortical basal degeneration.

35. A method of treating or preventing Alzheimer's Disease in a mammal, wherein the method comprises administering to the mammal a therapeutically effective amount of a compound or pharmaceutically acceptable salt thereof according to claim 1.

36. The method of claim 33, wherein the mammal is a human.

37. A method of treating or preventing an Aβ-related pathology in a mammal, wherein the method comprises administering to the mammal:
a therapeutically effective amount of a compound or pharmaceutically acceptable salt thereof according to claim 1; and
a cognitive enhancing agent, memory enhancing agent, or choline esterase inhibitor.
